(19) United States
(12) Patent Application Publication
(10) Pub. No.: US 2024/0344143 A1
(43) Pub. Date: Oct. 17, 2024

(54) ARRAYS TARGETING DIFFERENTIALLY ACCESSIBLE CHROMATIN REGIONS USING QUANTITATIVE POLYMERASE CHAIN REACTION

(71) Applicant: Episteme Prognostics, Inc., New York, NY (US)

(72) Inventor: Surajit DHARA, New York, NY (US)

(21) Appl. No.: 18/627,985

(22) Filed: Apr. 5, 2024

Related U.S. Application Data
(60) Provisional application No. 63/494,387, filed on Apr. 5, 2023.

(51) Int. Cl. C12Q 1/6886; C12Q 1/6809; C12Q 1/6816; C12Q 1/686; G01N 33/543; G01N 33/569; G01N 33/68

(52) U.S. Cl. CPC C12Q 1/6886; C12Q 1/6809; C12Q 1/6816; C12Q 1/686; G01N 33/54326; G01N 33/56966; G01N 33/6872; C12Q 2600/106; C12Q 2600/118; C12Q 2600/156; G01N 2333/4703

(57) ABSTRACT

The present disclosure relates to a quantitative PCR (qPCR) array-based assay for transposase-accessible chromatin and prognostic molecular markers of treatment-resistant/early recurrent cancer.

### 1. CROSS-REFERENCE OF RELATED APPLICATIONS

This application claims the benefit of and priority to U.S. Provisional Application No. 63/494,387, filed Apr. 5, 2023, which is hereby incorporated by reference in its entirety.

### 2. SEQUENCE LISTING

This instant application contains a Sequence Listing which has been submitted via Patent Center and is hereby incorporated by reference in its entirety. Said XML copy, created on Apr. 2, 2024, is named 55154WO_CRF_sequencelisting, and is 27,347 bytes in size.

### 3. TECHNICAL FIELD

The present disclosure relates to arrays targeting differentially accessible chromatin regions using, for example, quantitative polymerase chain reaction (qPCR), when the number of differentially represented regions are, for example, fewer than 100. The disclosure also relates to methods of using such arrays to, for example, guide chemotherapy or immunotherapy in cancer (e.g., gastrointestinal cancer and/or pancreatic ductal adenocarcinoma).

### 4. BACKGROUND

Pancreatic ductal adenocarcinoma (PDAC) is a lethal malignancy of the pancreas, with 66,440 new cases reported last year in the United States alone. By 2030, this disease is projected to surpass breast, prostate and colorectal cancer to become the second leading cause of cancer-related death in the United States. Almost 80% of patients are clinically presented as surgically non-resectable metastatic diseases. In the remaining resectable subset, the disease recurs in approximately 50% of cases within the first year of surgery despite adjuvant chemotherapy, another 30-40% recurs within next 2-5 years, whereas a small subset (10-15%) shows long-term disease-free survival (DFS) of more than 10 years.

Identification of patients at risk for recurrence, and particularly early recurrence in PDAC, and also in other types of cancers, in a timely manner is critical for reducing healthcare costs. Therefore, there is a need for approaches to identify such patients and tailor treatment accordingly.

### 5. SUMMARY

In one aspect, described herein are methods for determining a prognostic score indicative of a subject's responsiveness to one or more treatment modalities or indicative of a duration of disease-free survival, the method comprising:
(a) contacting a biological sample obtained from the subject with a transposase complex to produce a population of tagged DNA fragments representing accessible chromatin regions of the intact nuclei or intact nucleosome structure, wherein the biological sample comprises pancreatic ductal adenocarcinoma cells having morphologically intact nuclei or intact nucleosomes,
(b) hybridizing a set of targeting oligonucleotide probes to a specific region on the accessible chromatin regions to generate targeted accessible chromatin region fragments, wherein the targeting oligonucleotide probes specifically target differentially accessible chromatin regions, wherein the targeting oligonucleotide probes comprise:
   (i) a first subset of oligonucleotide probes targeting accessible chromatin regions associated with a first phenotype, and
   (ii) a second subset of oligonucleotide probes targeting accessible chromatin regions associated with a second phenotype;
(c) amplifying the targeted accessible chromatin region fragments obtained in (b) that are associated with the first phenotype and the second phenotype; and
(d) determining a prognostic score based on epigenetic signature values of the biological sample, wherein the prognostic score is determined based on a differential of a first epigenetic signature value and a second epigenetic signature value, wherein the prognostic score is indicative of the subject's responsiveness to the one or more treatment modalities or indicative of a duration of disease-free survival.

In one aspect, described are methods of treating a subject having, or suspected of having, pancreatic ductal adenocarcinoma with one or more treatment modalities, the method comprising:
(a) receiving a prognostic score indicative of the subject's responsiveness to one or more treatment modalities or indicative of a duration of disease-free survival, wherein the prognostic score is determined based on epigenetic signature values of a biological sample from the subject that comprises pancreatic ductal adenocarcinoma cells having morphologically intact nuclei or intact nucleosomes, the biological sample having been contacted with a transposase complex to produce a population of tagged DNA fragments representing accessible chromatin regions of the intact nuclei or intact nucleosome structure, wherein a set of targeting oligonucleotide probes were hybridized to a specific region on the accessible chromatin regions to generate targeted accessible chromatin region fragments, wherein the targeting oligonucleotide probes specifically target differentially accessible chromatin regions, wherein the prognostic score is determined based on a differential of a first epigenetic signature value and a second epigenetic signature value; and
(b) treating the subject with the one or more treatment modalities based on the prognostic score.

In accordance with any one of the embodiments, the method further comprising, prior to step (a), enriching the biological sample for tumor cells.

In accordance with any one of the embodiments, the method further comprising contacting the biological sample with an agent to isolate tumor cells from non-tumor cells in the biological sample to enrich the sample for tumor cells.

In some embodiments, the agent comprises antibody-conjugated magnetic beads, EpCAM-conjugated magnetic beads, or a combination thereof.

In accordance with any one of the embodiments, the method further comprising:
(i) attaching a detectable label to the tagged DNA fragments to produce labeled fragments; and
(ii) contacting the detectable labeled fragments to the set of targeting oligonucleotide probes.

In accordance with any one of the embodiments, the method further comprising quantifying the amount of amplified targeted accessible chromatin region fragments to obtain the first epigenetic signature value and the second epigenetic value.

In accordance with any one of the embodiments, the set of targeting oligonucleotide probes are selected from any pair of oligonucleotide probes in Table 4.

In accordance with any one of the embodiments, the targeting oligonucleotide probes comprises a nucleic acid sequence having a sequence of any one of SEQ ID NOs.: 1-30.

In accordance with any one of the embodiments, the set of targeting oligonucleotide probes comprises probes targeting at least one of ARHGEF10, CLDN23, C12orf36, and SPATA4.

In accordance with any one of the embodiments, the set of targeting oligonucleotide probes comprises probes targeting at least one of MAP2K2, PPAP2B, CNBP, FIG. 4, TTC19, PAPL, RN7SL300P, and ITGAV.

In accordance with any one of the embodiments, the set of targeting oligonucleotide probes comprises probes targeting at least one of GTF3C6, LINC01703, C1orf131, and XRCC2.

In accordance with any one of the embodiments, the accessible chromatin regions are selected from any one of the accessible chromatin regions in FIG. 9A, FIG. 9B, and/or Table 3.

In some embodiments, the accessible chromatin regions are selected from any one of the accessible chromatin regions in Table 3.

In accordance with any one of the embodiments, the method further comprising the step of comparing the first epigenetic signature value to the second epigenetic signature value to obtain a differential value.

In accordance with any one of the embodiments, the method further comprising normalizing the differential value with at least one of a positive control value and one of a negative control value to obtain the prognostic score.

In some embodiments, the prognostic score is at least 0.6.

In some embodiments, the method further comprising detecting nuclear localization of at least one of a transcription factor selected from any one of the transcription factors in Tables 2A and 2B.

In some embodiments, the transcription factors are selected from ZKSCAN1, EPAS1, RUNX2, ZNF410, MAFF, RREB1, NR3C2, SMAD1, RUNX1, ZNF32, ZSCAN4, HOXB1, POU3F1, ZBTB3, CLOCK, TCF15, GCM1, HINFP, CGBP, MYPOP, ZNF384, GMEB2, E2F5, AC012531.1, ZBTB7B, HOXC9, HNF4G, CREB1, ATF2, E2F2, SP3, ARID5A, ZFP161, OTP, PBX3, ZBTB33, ONECUT3, ONECUT3, DLX2, HNF4A, PRRX1, TCFL5, HOXB7, IRF6, GRHL1, FOXD2, ISL1, MLL, GATA2, GATA1, HMBOX1, NRF1, ZFHX3, ONECUT1, TET1, E2F3, DNMT1, CTCFL, CTCF, HNF1B, and HNF1A.

In some embodiments, the transcription factors are selected from ZKSCAN1A, HNF1B, or a combination thereof.

In accordance with any one of the embodiments, the method further comprising predicting a long duration of disease-free survival when the first epigenetic signature value is significantly higher than the second epigenetic signature value and/or predicting a short duration of disease-free survival when the second epigenetic signature value is significantly higher than the first epigenetic value.

In accordance with any one of the embodiments, the first phenotype is recurrence of a cancer within one year of surgical resection and the second phenotype is non-recurrence of a cancer within one year of surgical resection.

In accordance with any one of the embodiments, the first phenotype is non-responder and the second phenotype is responder to a cancer therapy.

In some embodiments, the cancer therapy is selected from chemotherapy, immunotherapy, radiation, or combinations thereof.

In accordance with any one of the embodiments, the second phenotype is having a median disease-free survival of between 50 to 1500 days.

In accordance with any one of the embodiments, the second phenotype is having a median disease-free survival of at least 350 days.

In accordance with any one of the embodiments, the first phenotype is having a median disease-free survival of between 1 to 350 days.

In accordance with any one of the embodiments, the first phenotype is having a median disease-free survival of less than 50 days.

In accordance with any one of the embodiments, the biological sample comprises treatment-naïve malignant cells.

In accordance with any one of the embodiments, the subject is a treatment naïve patient who has not received the one or more treatment modalities.

In accordance with any one of the embodiments, the subject is under treatment or has been treated with the one or more treatment modalities.

In accordance with any one of the embodiments, the one or more treatment modalities are selected from resecting cancerous tissue, neo-adjuvant chemotherapy, adjuvant chemotherapy, immunotherapy, an epigenetic drug, or combinations thereof.

In some embodiments, the epigenetic drug is selected from DNMT inhibitor, an HDAC inhibitor, an EZH2 inhibitor, or combinations thereof.

In some embodiments, the neo-adjuvant chemotherapy and/or adjuvant chemotherapy is selected from gemcitabine, nab-paclitaxel, fluorouracil (5-FU), irinotecan, oxaliplatin, leucovorin, capecitabine, cisplatin, or combinations thereof.

In accordance with any one of the embodiments, the biological sample is selected from a tumor biopsy or surgically resected tumor specimen.

In accordance with any one of the embodiments, the method does not include sequencing the tagged fragments or amplicons thereof. In accordance with any one of the embodiments, the amplified targeted accessible chromatin fragments comprise a mean size of about 120 bp.

In accordance with any one of the embodiments, obtaining the targeted accessible chromatin region fragments does not include the step of sequencing the tagged fragments or amplicons thereof.

In accordance with any one of the embodiments, the set of targeting oligonucleotide probes required for effectively determining the prognostic score is less than 100, 90, 80, 70, 60, 50, 40, 30, 20, or 10.

In some embodiments, the set of targeting oligonucleotide probes required for effectively determining the prognostic score is about 12.

In some embodiments, the set of targeting oligonucleotide probes required for effectively determining good prognosis or poor prognosis is less than 100, 90, 80, 70, 60, 50, 40, 30, 20, or 10.

In some embodiments, the set of targeting oligonucleotide probes required for effectively determining good prognosis is about 3.

In some embodiments, the set of targeting oligonucleotide probes required for effectively determining poor prognosis is about 8.

In one aspect, described are kits for determining an epigenetic landscape associated with a specific phenotypic trait of a biological sample, the kit comprising:
(a) an array configured to detect targeted accessible chromatin region fragments obtained from the biological sample, wherein the array comprises a panel of sets of targeting oligonucleotide probes specifically targeting differentially accessible chromatin regions;
(b) reagents; and
(c) instructions for amplifying and quantifying the targeted accessible chromatin region fragments to obtain a first epigenetic signature value and a second epigenetic value.

In some embodiments, the panel of targeting oligonucleotide probes is arranged as described in FIG. 4A or 4B.

In one aspect, described are kits for determining an epigenetic landscape associated with a specific phenotypic trait of a biological sample, the kit comprising:
(a) one or more sets of targeting oligonucleotide probes for detect targeted accessible chromatin region fragments obtained from the biological sample, wherein the targeting oligonucleotide probes specifically target and amplify differentially accessible chromatin regions to generate one or more targeted accessible chromatin region fragments;
(b) reagents; and
(c) instructions for amplifying and quantifying the targeted accessible chromatin region fragments to obtain a first epigenetic signature value and a second epigenetic value.

In accordance with any one of the embodiments, the biological sample comprises pancreatic ductal adenocarcinoma cells having morphologically intact nuclei or intact nucleosome (histone-DNA) structure.

In accordance with any one of the embodiments, the kit further comprising reagents and instruction for obtaining the biological sample by contacting the morphologically intact nuclei or intact nucleosome (histone-DNA) structure to a transposase complex to produce a population of tagged DNA fragments representing the targeted accessible chromatin region fragments.

In accordance with any one of the embodiments, the targeting oligonucleotide probes comprises a nucleic acid sequence having a sequence of any one of SEQ ID NOs.: 1-30.

In accordance with any one of the embodiments, the set of targeting oligonucleotide probes comprises probes targeting at least one of ARHGEF10, CLDN23, C12orf36, and SPATA4.

In accordance with any one of the embodiments, the set of targeting oligonucleotide probes comprises probes targeting at least one of MAP2K2, PPAP2B, CNBP, FIG. 4, TTC19, PAPL, RN7SL300P, and ITGAV.

In accordance with any one of the embodiments, the set of targeting oligonucleotide probes comprises probes targeting at least one of GTF3C6, LINC01703, C1orf131, and XRCC2.

In accordance with any one of the embodiments, further comprising reagents and instructions for enriching the biological sample obtained from the subject for tumor cells.

In some embodiments, the reagents comprise antibody-conjugated magnetic beads, EpCAM-conjugated magnetic beads, or a combination thereof to isolate tumor cells from non-tumor cells in the biological sample.

In accordance with any one of the embodiments, the kit further comprising reagents and instructions for:
(i) attaching a detectable label to the tagged DNA fragments to produce labeled fragments; and
(ii) contacting the detectable labeled fragments to the set of oligonucleotide probes.

In accordance with any one of the embodiments, the kit further comprising instructions for determining a prognostic score indicative of the subject's responsiveness to one or more treatment modalities based on a differential score of the first epigenetic signature value and the second epigenetic value.

In accordance with any one of the embodiments, the instruction comprises normalizing the differential value with at least one of a positive control value and a negative control value to obtain the prognostic score.

In some embodiments, the prognostic score is at least 0.6.

In accordance with any one of the embodiments, the kit further comprising reagents and instructions for detecting nuclear localization of a transcription factor selected from any one of transcription factors in Tables 2A and 2B.

In some embodiments, the transcription factors are selected from ZKSCAN1, EPAS1, RUNX2, ZNF410, MAFF, RREB1, NR3C2, SMAD1, RUNX1, ZNF32, ZSCAN4, HOXB1, POU3F1, ZBTB3, CLOCK, TCF15, GCM1, HINFP, CGBP, MYPOP, ZNF384, GMEB2, E2F5, AC012531.1, ZBTB7B, HOXC9, HNF4G, CREB1, ATF2, E2F2, SP3, ARID5A, ZFP161, OTP, PBX3, ZBTB33, ONECUT3, ONECUT3, DLX2, HNF4A, PRRX1, TCFL5, HOXB7, IRF6, GRHL1, FOXD2, ISL1, MLL, GATA2, GATA1, HMBOX1, NRF1, ZFHX3, ONECUT1, TET1, E2F3, DNMT1, CTCFL, CTCF, HNF1B, and HNF1A.

In some embodiments, the transcription factors are selected ZKSCAN1A, HNF1B, or a combination thereof.

In accordance with any one of the embodiments, the kit further comprising reagents and instructions for predicting a long duration of disease-free survival when the first epigenetic signature value is significantly higher than the second epigenetic signature value and/or predicting a short duration of disease-free survival when the second epigenetic signature value is significantly higher than the first epigenetic value.

In accordance with any one of the embodiments, the kit further comprising reagents and instructions for determining a first phenotype and second phenotype of the subject's responsiveness to one or more treatment modalities.

In some embodiments, the first phenotype is recurrence of a cancer within one year of surgical resection and the second phenotype is non-recurrence of a cancer within one year of surgical resection.

In some embodiments, the first phenotype is non-responder and the second phenotype is responder to a cancer therapy.

In some embodiments, the cancer therapy is selected from chemotherapy, immunotherapy, radiation, or combinations thereof.

In accordance with any one of the embodiments, the second phenotype is having a median disease-free survival of between 50 to 1500 days.

In accordance with any one of the embodiments, the second phenotype is having a median disease-free survival of at least 350 days.

In accordance with any one of the embodiments, the first phenotype is having a median disease-free survival of between 1 to 350 days.

In accordance with any one of the embodiments, the first phenotype is having a median disease-free survival of less than 50 days.

In accordance with any one of the embodiments, the biological sample comprises treatment-naïve malignant cells.

In some embodiments, the subject is a treatment naïve patient who has not received the one or more treatment modalities.

In accordance with any one of the embodiments, the subject is under treatment or has been treated with the one or more treatment modalities.

In accordance with any one of the embodiments, the one or more treatment modalities are selected from resecting cancerous tissue, neo-adjuvant chemotherapy, adjuvant chemotherapy, immunotherapy, an epigenetic drug, or combinations thereof.

In some embodiments, the epigenetic drug is selected from DNMT inhibitor, an HDAC inhibitor, an EZH2 inhibitor, or combinations thereof.

In some embodiments, the neo-adjuvant chemotherapy and/or adjuvant chemotherapy is selected from gemcitabine, nab-paclitaxel, fluorouracil (5-FU), irinotecan, oxaliplatin, leucovorin, capecitabine, cisplatin, or combinations thereof.

In accordance with any one of the embodiments, the biological sample is selected from a tumor biopsy or surgically resected tumor specimen.

In accordance with any one of the embodiments, obtaining the targeted accessible chromatin region fragments does not include sequencing the tagged fragments or amplicons thereof.

In accordance with any one of the embodiments, the amplified targeted accessible chromatin fragments comprise a mean size of about 120 bp.

In accordance with any one of the embodiments, the set of targeting oligonucleotide probes required for effectively determining the prognostic score is less than 100, 90, 80, 70, 60, 50, 40, 30, 20, or 10.

In some embodiments, the set of targeting oligonucleotide probes required for effectively determining the prognostic score is about 12.

In some embodiments, the set of targeting oligonucleotide probes required for effectively determining good prognosis or poor prognosis is less than 100, 90, 80, 70, 60, 50, 40, 30, 20, or 10.

In some embodiments, the set of targeting oligonucleotide probes required for effectively determining good prognosis is about 3.

In some embodiments, the set of targeting oligonucleotide probes required for effectively determining poor prognosis is about 8.

In one aspect, this disclosure provides a low-cost array targeting fewer than 100 differentially accessible chromatin regions by qPCR. The differentially accessible chromatin regions may be identified using Assay for Transposase-Accessible Chromatin-sequencing (ATAC-seq) or Assay for Transposase-Accessible Chromatin-array (ATAC-array); the array may be a “targeted ATAC-qPCRarray.” Such arrays, unlike ATAC-seq or ATAC-array, detect only the “targeted” accessible chromatin regions of interest, particularly valuable when the differentially accessible regions are fewer than 100. ATAC-array, a hybridization-based reading of chromatin accessibility suitable for reading the chromatin signature more than 100 regions is described in WO2020036929A1, or US20210324376A1, or Dhara et al. Pancreatic cancer prognosis is predicted by an ATAC-array technology for assessing chromatin accessibility. Nat Commun 12, 3044 (2021), or by ATAC-seq as described in Shin et al. Chromatin accessibility of circulating CD8⁺ T cells predicts treatment response to PD-1 blockade in patients with gastric cancer. Nat Commun 12, 975 (2021), each of which is incorporated herein by reference in its entirety.

In one aspect, this disclosure provides methods for guiding cancer treatment (e.g., guiding chemotherapy and/or immunotherapy). In certain embodiments, an array disclosed herein is used to guide cancer treatment. For example, an array can be a prognostic tool in the field of precision oncology, associating a specific set of open chromatin regions of the functional genome with specific disease phenotypes or response to a specific regimen of drug, or combination of drugs. Although gene expression signatures associated with prognosis have been described in malignant diseases, unlike gene expression signature, which is an RNA-based technology, chromatin accessibility signature is a DNA-based technology therefore more robust and better applicability in routine clinical specimens.

In one aspect, described herein are methods for determining an epigenetic landscape associated with a specific phenotypic trait of a biological sample, the method comprising: (a) providing a biological sample comprising morphologically intact nuclei or intact nucleosome (histone-DNA) structure; (b) contacting the intact nuclei or intact nucleosome to a transposase complex to produce a population of tagged DNA fragments representing accessible chromatin regions (ACRs) of the intact nuclei and/or intact nucleosome (histone-DNA) structure; (c) hybridizing a set of targeting oligonucleotide probes to a specific region on the ACRs to generate targeted ACR fragments, wherein the targeting oligonucleotide probes specifically targets no more than 100 differentially accessible chromatin regions selected from Table 1 or Table 2, wherein the targeting oligonucleotide probes comprise: (i) a first subset of oligonucleotide probes targeting ACRs associated with a first phenotype, and (ii) a second subset of oligonucleotide probes targeting ACRs associated with a second phenotype; (d) amplifying the targeted ACR fragments associated with the first phenotype and the second phenotype obtained in (c); and (c) quantifying the amount of amplified targeted ACR fragments, thereby determining the epigenetic landscape associated with the first and the second phenotypic traits of the biological sample. In some embodiments, the amplification fragment comprises a mean size of less than 100 bp.

In accordance with any of the embodiments, the first phenotype is recurrence of a cancer within one year of surgical resection and the second phenotype is non-recurrence of a cancer within one year of surgical resection.

In accordance with any of the embodiments, the first phenotype is non-responder, and the second phenotype is responder to a cancer therapy. In some embodiments, the cancer therapy is selected from chemotherapy, immunotherapy, and radiation.

In accordance with any of the embodiments, the method further comprises assessing nuclear localization of one or more biomarkers capable of modulating gene expression through complementary binding to one or more specific regions on the amplified targeted ACR fragments. In some embodiments, the biomarker is a transcription factor. In some embodiments, the transcription factor is selected from ZKSCAN1, EPAS1, RUNX2, ZNF410, MAFF, RREB1, NR3C2, SMAD1, RUNX1, ZNF32, ZSCAN4, HOXB1, POU3F1, ZBTB3, CLOCK, TCF15, GCM1, HINFP, CGBP, MYPOP, ZNF384, GMEB2, E2F5, AC012531.1, ZBTB7B, HOXC9, HNF4G, CREB1, ATF2, E2F2, SP3, ARID5A, ZFP161, OTP, PBX3, ZBTB33, ONECUT3, ONECUT3, DLX2, HNF4A, PRRX1, TCFL5, HOXB7, IRF6, GRHL1, FOXD2, ISL1, MLL, GATA2, GATA1, HMBOX1, NRF1, ZFHX3, ONECUT1, TET1, E2F3, DNMT1, CTCFL, CTCF, HNF1B, and HNF1A.

In accordance with any of the embodiments, the biological sample is selected from a tumor biopsy, surgically resected tumor specimen, liquid biopsy, bodily fluid, blood, plasma, saliva, semen, vaginal discharge, urine, circulating shedding tumor tissue, and/or circulating tumor DNA (ctDNA).

In accordance with any of the embodiments, the biological sample is pancreatic ductal adenocarcinoma tissue.

In accordance with any of the embodiments, the biological sample comprises treatment-naïve malignant cells.

In accordance with any of the embodiments, the biological sample is collected from a patient who had been treated with one or more treatment modalities.

In accordance with any of the embodiments, the phenotypic trait is responsiveness to a treatment modality. In some embodiments, the treatment modality is selected from the group consisting of surgical resection, chemotherapy, radiation, immunotherapy, and a combination thereof.

In accordance with any of the embodiments, the method further comprises isolating nucleosomal DNA from the cell nuclei of the biological sample. In some embodiments, the nuclei are isolated and in a manner that maintains nucleosome structure.

In accordance with any of the embodiments, the method does not include sequencing the tagged fragments or amplicons thereof.

An epigenetic landscape integrates the entire ensemble of epigenetic silencing and/or activating events in the genome (e.g., through methylation and acetylation together). In certain embodiments, the epigenetic landscape is assessed by a qPCR-based platform described herein, generally referred to as “ATAC-qPCRarray.” One exemplary application of the ATAC-qPCRarray technology is as a diagnostic test that can be performed on tumor biopsies or surgically resected tumor specimens, or liquid biopsies. Typically, results are provided within a day.

In an exemplary embodiment, an epigenetic landscape is significantly associated with prognosis and, in particular, early disease recurrence (i.e., within 1 year of surgery) in PDAC patients even after apparently complete surgical removal (RO margin-negative resection) of the primary tumor, and in spite of adjuvant chemotherapy (e.g., gemcitabine). The epigenetic landscape may comprise at least 700 functionally relevant regulatory regions silenced and at least 300 other functionally relevant regulatory regions accessible in patients who did not respond to their first-line of chemotherapy (gemcitabine). Another exemplary embodiment, an epigenetic landscape is predictive of immunotherapy (immune checkpoint inhibition (ICI) therapy). The epigenetic landscape may comprise at 67 functionally relevant regulatory regions predicts with high sensitivity (100.0%) and specificity (90.9%) for distinguishing responder from non-responder to ICI drug pembrolizumab.

### 6. BRIEF DESCRIPTION OF THE DRAWINGS

For a better understanding of the invention, reference may be made to embodiments shown in the following drawings. The components in the drawings are not necessarily to scale and related elements may be omitted, or in some instances proportions may have been exaggerated, so as to emphasize and clearly illustrate the novel features described herein. In addition, system components can be variously arranged, as known in the art.

FIGS. 1A-1B illustrate representation of the 1092 differentially accessible chromatin peaks identified by ATAC-seq in pancreatic cancer patients (FIG. 1B) from the near-saturation identification of 121697 open chromatin peaks across the genome (FIG. 1A).

FIGS. 2A-2B depict a schematic representation of an ATAC-array (FIG. 2A) and the correlation of the ATAC-seq (FIG. 2B) confined to the signature regions.

FIGS. 3A-3B depict histogram results of the ATAC-array showing the differential enrichment of peaks from a poor-prognosis (FIG. 3A) and better-prognosis (FIG. 3B) pancreatic cancer patient.

FIGS. 4A-4B depict a schematic representation of an exemplary ATAC-qPCRarray approach described herein, where (FIG. 4A) 96-well format and (FIG. 4B) is 384-well format.

FIG. 5 depicts sixty (60) TFs identified whose motifs were differentially open in recurrent (17 motifs) and non-recurrent (44 motifs) patients.

FIG. 6 provides a K-M graph showing significant segregation of good prognosis and poor prognosis. P value=0.0019.

FIGS. 7A and 7B provide results of melting curve analysis showing a single peak for each primer set.

FIG. 8 shows K-M graph showing the long-term (n=7) and short-term (n=7) survivor patients selected for the validation of the targets. P value=0.00014.

FIGS. 9A and 9B show results from ATAC-qPCR analysis of the 11 target regions normalized with the 4 control regions.

FIG. 10 shows Box plot showing the significant difference (t test p=0.034) in PS between good and poor prognosis patients. P value=0.034.

### 7. DETAILED DESCRIPTION

This detailed description is intended only to acquaint others skilled in the art with the present invention, its principles, and its practical application so that others skilled in the art may adapt and apply the invention in its numerous forms, as they may be best suited to the requirements of a particular use. This description and its examples are intended for purposes of illustration only. This invention, therefore, is not limited to the embodiments described in this patent application, and may be variously modified.

### 7.1 Definitions

As used in the specification and the appended claims, unless specified to the contrary, the following terms have the meaning indicated:

The term “about” as used herein, means approximately, and in most cases within 10% of the stated value.

The term “qPCRarray” is intended to describe a two-dimensional or three-dimensional arrangement of addressable regions bearing oligonucleotides associated with that region. The accessible regions from an ATAC library prepared from specimens will be amplified by quantitative polymerase chain reaction with a set of specifically designed oligonucleotide primers complementary to the accessible regions (signatures with high predictive ability of prognosis and/or drug response)

The term “biological sample” is to be understood as any in vivo, in vitro, or in situ sample of one or more cells or cell fragments. This can, for example, be a unicellular or multicellular organism, blood sample, biopsied tissue sample, tissue section, cytological sample, or any derivative of the foregoing (e.g., a subsample, portion, or purified cell population). In certain embodiments, a biological sample is obtained from a mammal, including, but not limited to, a primate (including human), mouse, rat, cat, or dog.

The term “cancer” includes, but is not limited to, breast cancer, colorectal cancer, esophageal cancer, gallbladder cancer, gastric cancer, leukemia (e.g., acute myeloid leukemia (AML) or chronic myeloid leukemia (CML)), liver cancer (e.g., hepatocellular carcinoma (HCC)), lung cancer (e.g., non-small cell lung cancer (NSCLC) or small cell lung cancer (SCLC)), lymphoma (e.g., non-Hodgkin lymphoma), ovarian cancer, pancreatic cancer, and prostate cancer, The term “cancer” also includes cancer metastasis of a primary tumor such as primary pancreatic cancer. Thus, if reference is made, for example, to pancreatic cancer, this also includes metastasis of the pancreatic cancer, for example metastasis to the lung, liver and/or lymph nodes.

The term “detectable label” refers to a moiety that can be attached directly or indirectly to an oligomer, such as an oligonucleotide, to thereby render the oligomer detectable by an instrument or method.

The term “transposase complex” refers to a complex that contains a transposase (which typically exists as a dimer of transposase polypeptides) that is bound to at least one adapter. The term “adapter” refers to a nucleic acid molecule that is capable of being attached to a polynucleotide of interest. An adapter can be single stranded or double stranded, and it can comprise DNA, RNA, and/or artificial nucleotides. The adapter can add one or more functionalities or properties to the polynucleotide of interest, such as providing a priming site for amplification or adding a barcode. By way of example, adapters can include a universal priming site for amplification. By way of further example, adapters can one or more barcode of various types or for various purposes, such as molecular barcodes, sample barcodes and/or target-specific barcodes. In practice, a transposase complex can be used to attach an adapter to the end of a DNA fragment generated by the enzymatic action of the transposase.

The terms “treat”, “treating” and “treatment” refer to a method of alleviating or abrogating a condition, disorder, or disease and/or the attendant symptoms thereof.

The term “accessible chromatin regions” or “ACR” or “open chromatin regions” refer to regions of DNA within a cell's nucleus that are loosely packaged and available to proteins involved in transcription.

The terms “chromatin accessibility patterns” refers to “open chromatin” or “close chromatin” patterns.

The term “open chromatin” or “euchromatin” refers to regions of DNA within the nucleus that are loosely packaged and readily accessible to transcription. The regions are typically associated with genes that are expressed or active within a cell.

The term “close chromatin” or “heterochromatin” refers to regions of DNA within the cell's nucleus being tightly packaged, which hinders the access of proteins for transcription. The regions are typically associated with genes that are repressed are inactive within a cell.

The term “chromatin accessibility signature” refers to a defined set of accessible chromatin regions (ACRs) that are differentially represented, showing a defined pattern (signature) statistically associated (or correlated) with a specific phenotype. For example, as published in Dhara et al., Nature Communications 2021, 1092 differentially represented ACRs are correlated with pancreatic cancer patients who responded or non-responded to chemotherapy.

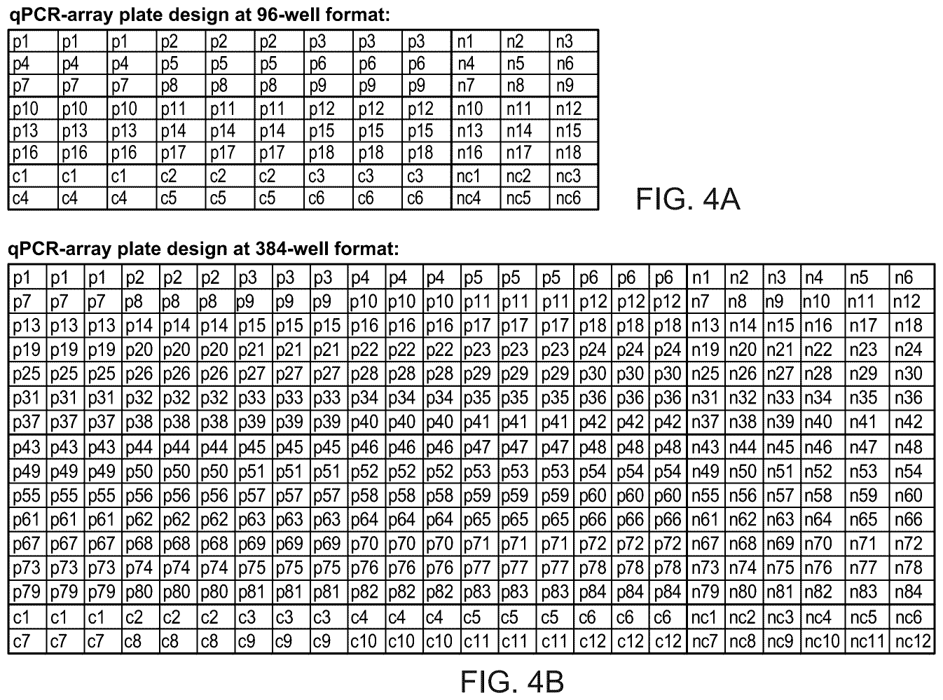

The term “epigenetic signature value” refers to a median value of a set of accessible chromatin regions indicating a good prognosis (e.g., set of blue regions) or a median value of a set of accessible chromatin regions indicating a poor prognosis (e.g., set of red regions). For example, the median prognosis value of a set of accessible chromatin regions indicating a good prognosis is the median value of at least 3 replications of ATAC-qPCR array (e.g., the median Ct value) of the accessible chromatin regions selected from at least one of CLDN23, SPATA4, ARHGEF-10, and C12orf36. Conversely, the median prognosis value of a set of accessible chromatin regions indicating a poor prognosis is the median value of at least 3 replications of ATAC-qPCR array analyses (e.g., the median Ct value) of the accessible chromatin regions selected from at least one of RN7SL, CNBP, MAP2K2, ITGAV, PPAP2B, FIG. 4, PAPL, and TTC19. A control epigenetic signature value refers to the median control value, which is the median prognosis value of a set of accessible chromatin regions indicating the median value of at least 3 replications of ATAC-qPCR array analyses (e.g., the median Ct value) of the accessible chromatin regions selected from at least one of GTF3C6, LINC01703, C1orf131, and XRCC2.

As used herein, the term “Ct value” refers to a cycle threshold or the number of cycles needed in a polymerase chain reaction (PCR) test to amplify a specific target molecule to a detectable level. In some embodiments, the Ct value indicates the amount of starting materials present in the sample. In the context of detecting accessible chromatin regions, the Ct value can reflect, for example, the availability of the chromatin region for transcription factor binding. A lower Ct value indicates fewer PCR cycles needed and can indicate a higher availability of accessible chromatin regions. A higher Ct value indicates more PCR cycles needed and can indicate a lower availability of accessible chromatin regions. In a qPCR analysis, a low 2\hat{ }⁻dCt reflects fewer PCR cycles are needed to detect the target molecule (e.g., accessible chromatin region) in the sample and can indicate a higher initial amount or availability of the target molecule present in the sample. In the context of detecting accessible chromatin regions, a low 2\hat{ }⁻dCt can represent a high relative abundance of the accessible chromatin region in the sample compared to the reference sample (e.g., control).

The term “prognostic score (PS)” refers to the difference between an epigenetic signature value of a good prognosis accessible chromatin region and an epigenetic signature value of a poor prognosis accessible chromatin region, and the difference is normalized with a control epigenetic signature value as illustrated in Section 7.2 and Example 5 herein. The prognostic score can be used to assess, determine, or predict a patient's responsiveness to a treatment modality (e.g., chemotherapy, immunotherapy, or surgery). For example, a prognostic score of >0.6 indicates the patient has a good response to the treatment, while a prognostic of <0.6 indicates the patient has a poor prognosis to the treatment.

The term “differential value” refers to the difference between an epigenetic signature value of a good prognosis and an epigenetic signature value of poor prognosis. The differential value can be used to determine the prognostic scores with a range of values, for example, <0.6 non-responder indicates poor prognosis and >0.6 responder indicates good prognosis.

The term “responder” refers to a patient who has good responsiveness to a treatment modality described herein. A responder may have a DFS of at least 50 days, at least 400 days, or longer.

The term “non-responder” refers to a patient who has poor responsiveness to a treatment modality described herein. A non-responder may have a DFS of less than 400 days, less than 100 days, less than 50 days, or shorter.

In this application, the use of the disjunctive is intended to include the conjunctive. The use of definite or indefinite articles is not intended to indicate cardinality. In particular, a reference to “the” object or “a” and “an” object is intended to denote also one of a possible plurality of such objects. Further, the conjunction “or” may be used to convey features that are simultaneously present instead of mutually exclusive alternatives. In other words, the conjunction “or” should be understood to include “and/or.” The terms “includes,” “including,” and “include” are inclusive and have the same scope as “comprises,” “comprising,” and “comprise” respectively.

### 7.2 QPCR Array Methods

In one aspect, the present disclosure provides a method for analyzing chromatin accessibility. Chromatin may be present in the nuclei or in samples in which nucleosomal structure has been maintained (e.g., a product of lysed nuclei, or circulating tumor DNA (ctDNA)). In certain embodiments, the method comprises: (a) providing a biological sample comprising chromatin, such as from morphologically intact nuclei; (b) fragmenting and tagging accessible chromatin regions (ACRs) to produce tagged fragments; (c) optionally, amplifying the tagged fragments; (d) hybridizing or attaching a set of oligonucleotide primers specifically designed to the ACRs; and (c) amplifying the ACRs by PCR master mix containing SYBR-green followed by detection of fluorescence intensities of SYBR-green by qPCR machine. In some embodiments, fragmenting and tagging ACRs comprising using enzymatic tagmentation such as using Tn transposase-based or restriction enzyme-based tagmentation. In some embodiments, fragmenting and tagging ACRs comprising using enzymatic tagmentation such as sonication, microfluidic shearing, or chemical cleavage (e.g., hydrogen peroxide, potassium permanganate). In certain embodiments, the method further comprises determining the accessibility of at least one chromatin region. In certain embodiments, the set of oligonucleotide probes represent chromatin regions that are differentially accessible between a first phenotype and a second phenotype (e.g., between treatment-resistant disease and treatment-sensitive disease; between a cancer likely to recur within one year following surgical resection and a cancer likely not to recur within one year following surgical resection). In some such embodiments, the set of oligonucleotide primers comprises (i) a first subset of oligonucleotide primers representative of accessible chromatin regions associated with the first phenotype and (ii) a second subset of oligonucleotide primers representative of accessible chromatin regions associated with the second phenotype.

In certain embodiments, the method does not include sequencing the tagged fragments or amplicons thereof.

In certain embodiments, at least some of the differentially accessible chromatin regions include a promoter, an enhancer, and/or other regulatory elements. In certain embodiments, the biological sample comprises malignant or diseased tissue. In other embodiments, the biological sample comprises normal tissue.

In certain embodiments, the method comprises providing a biological sample. The biological sample may be, for example, a blood sample, a tissue sample, or a cytological sample. In certain embodiments, the biological sample comprises cancerous cells or cells suspected of being cancerous. In some such embodiments, the biological sample is unprocessed. In other such embodiments, the biological sample is processed to, for example, isolate a specific cell population. For example, a population of Epithelial Cell Adhesion Molecule positive (EpCAM+) cells (i.e., cells expressing the transmembrane protein EpCAM) may be isolated from a tissue sample such as tissue biopsied from a pancreatic tumor or, more specifically, a pancreatic ductal adenocarcinoma, or CD8+T-lymphocyte cells from peripheral blood of a patient. Without being bound to any theory, cancer cells may express more EpCAM compared to healthy cells. Isolating cells expressing EpCAM may enrich and isolate tumor cells from the biological sample.

In certain embodiments, the biological sample can be obtained from a patient diagnosed with cancer. For example, in case of pancreatic cancer a patient may be referred to undergo endoscopic ultrasound and fine needle aspiration (EUS-FNA) for tissue diagnosis of a suspected pancreatic mass, which may result in the diagnosis of PDAC. This EUS-FNA or the laparoscopic surgery tissue acquisition process occurs prior to chemotherapy treatment and may provide treatment-naïve malignant cells from all stages of PDAC.

In certain embodiments, the method further comprises isolating nucleosomal DNA from the biological sample, such as an isolated cell population, or peripheral blood. In some such embodiments, nuclei are isolated and/or lysed in a manner that maintains nucleosome structure.

Nuclei are isolated or collected in such a manner as to ensure that nucleosomal structure is maintained. Thus, nuclei comprise regions of tightly packed or closed chromatin and regions of loosely packed or open chromatin. In certain embodiments, the method comprises fragmenting open chromatin regions of nuclei to obtain a population of fragments representing the open chromatin regions. In certain embodiments, the method comprises tagging such fragments with, for example, an adapter. In certain embodiments, the fragmenting and tagging occurs substantially simultaneously or in rapid succession. Certain transposases such as a hyperactive Tn5 transposase, loaded in vitro with adapters, can substantially simultaneously fragment and tag DNA with the adapters. Thus, in some embodiments, the method may comprise “tagmenting” the open chromatin regions using, for example, a hyperactive Tn5 transposase loaded with one or more adapters.

In certain embodiments, the fragmenting and tagging step comprises contacting morphologically intact nuclei with a transposase complex. In some such embodiments, a transposase complex comprises a transposase enzyme (which is usually in the form of a dimer of transposase polypeptides) and a pair of adapters. In certain embodiments, isolated nuclei are lysed when contacted with a transposase complex and, thus, the method may comprise lysis of intact nuclei.

In certain embodiments, the transposase is prokaryotic, eukaryotic, or from a virus. In certain embodiments, the transposase is a hyperactive transposase. In certain embodiments, the transposase is an RNase transpose, such as a Tn transposase. In some such embodiments, the transposase is a Tn5 transposase or derived from a Tn5 transposase. In certain preferred embodiments, the transposase is a hyperactive Tn5 transposase (e.g., a Tn5 transposase having an L372P mutation). In certain embodiments, the transposase is a MuA transposase or derived from a MuA transposase. In certain embodiments, the transposase is a Vibhar transposase (e.g., from Vibrio harveyi) or derived from a Vibhar transposase. In the above examples, a transposase derived from a parent transposase can comprise a peptide fragment with at least about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% amino acid sequence homology and/or identity to a corresponding peptide fragment of the parent transposase. The peptide fragment can be at least about 10, about 15, about 20, about 25, about 30, about 35, about 40, about 45, about 50, about 60, about 70, about 80, about 90, about 100, about 150, about 200, about 250, about 300, about 400, or about 500 amino acids in length. For example, a transposase derived from Tn5 can comprise a peptide fragment that is 50 amino acids in length and about 80% homologous to a corresponding fragment in a parent Tn5 transposase.

In an exemplary method described herein, the transposase complex comprises a transposase loaded with two adapter molecules that each contain a recognition sequence at one end. The transposase catalyzes substantially simultaneous fragmenting of the sample and tagging of the fragments with sequences that are adjacent to the transposon recognition sequence (i.e., “tagmentation”). In some cases, the transposase enzyme can insert the nucleic acid sequence into the polynucleotide in a substantially sequence-independent manner. In certain embodiments, a preliminary step includes loading a transposase with one or more oligonucleotide adapters. Typically, the adapters comprise oligonucleotides that have been annealed together so that at least the transposase recognition sequence is double stranded.

In certain embodiments, the amplifying step comprises an amplification reaction that results in a relatively uniform amplification of substantially all template sequences in a sample (e.g., at least 85%, 90%, or 95% of the template sequences). In certain embodiments, the amplifying step comprises polymerase chain reaction (PCR). In certain embodiments, the amplifying step comprises PCR using primers specific for adapter sequences appended to the fragments during the fragmenting and tagging step.

Results from the reading or evaluating may be raw results (such as fluorescence intensity readings for each feature in one or more color channels) or may be processed results (such as those obtained by subtracting a background measurement, or by rejecting a reading for a feature which is below a predetermined threshold, normalizing the results, and/or forming conclusions based on the pattern read from the array (such as whether or not a particular target sequence may have been accessible in the sample, or whether or not a pattern indicates a particular condition of an organism from which the sample came).

In one aspect, the present disclosure provides a method for determining an epigenetic landscape of a biological sample. In certain embodiments, the method comprises: (a) providing a biological sample obtained from a patient, said biological sample comprising morphologically intact nuclei; (b) contacting the morphologically intact nuclei to a transposase complex to produce a population of tagged DNA fragments representing accessible chromatin regions (ACRs) of the morphologically intact nuclei; (c) attaching a set of oligonucleotide primers to produce amplicon fragments; and (d) set of primers amplify the specifically targeted ACRs with qPCR master mix containing SYBR-green for quantitation of amplified DNA fragments. In certain embodiments, the method does not include sequencing, or hybridization of the tagged fragments or amplicons thereof.

In one aspect, the present disclosure provides a method for comparing epigenetic landscapes between a test sample and a reference sample. In certain embodiments, the method comprises: (a) analyzing morphologically intact nuclei from the test sample to produce a first epigenetic landscape; (b) analyzing morphologically intact nuclei from the reference sample to produce a second epigenetic landscape; and (c) comparing the first epigenetic landscape to the second epigenetic landscape. In certain embodiments, the test sample and the reference sample can be obtained from the same individual at different times (e.g., before and after treatment). In other embodiments, the test sample and the reference sample can be obtained from different individuals (e.g., a cancer patient and a subject without cancer; a cancer patient with treatment-resistant cancer and a cancer patient with treatment-sensitive cancer; or a cancer patient with an unknown diagnosis/prognosis and a cancer patient with treatment-resistant—or, alternatively, treatment-sensitive-cancer). In certain embodiments, the morphologically intact nuclei from the test sample and/or from the reference sample are analyzed according to a method described herein, such as by an ATAC-qPCRarray approach.

In one aspect, the present disclosure provides a method for identifying an epigenetic landscape characteristic of resistance to a cancer treatment modality. In certain embodiments, the method comprises (a) providing a first sample comprising cells from a treatment-resistant tumor (e.g., a recurrent pancreatic ductal adenocarcinoma, where the recurrence is within one year of resection) and a second sample comprising non-cancerous cells or tumor cells from a treatment-sensitive tumor (e.g., a non-recurrent pancreatic ductal adenocarcinoma or a late recurrent pancreatic ductal adenocarcinoma, where the recurrence is beyond 2 and up to 5 years after resection); (b) identifying accessible chromatin regions (ACRs) in both samples; and (c) comparing the ACRs identified in the first sample to the ACRs identified in the second sample. In certain embodiments, the epigenetic landscape characteristic of resistance to treatment comprises one or more ACRs present in first sample and not present in the second sample and/or one or more ACRs present in second sample and not present in the first sample. In certain embodiments, the cancer is pancreatic cancer. Pancreatic cancer includes, for example, adenocarcinomas (tumors exhibiting glandular architecture) arising within the exocrine component of the pancreas and neuroendocrine carcinomas arising from islet cells. Pancreatic ductal adenocarcinoma (PDAC) is the most common form of pancreatic cancer. Other forms of pancreatic cancer include mucinous adenocarcinoma, acinic cell neoplasm, and neuroendocrine carcinoma. In certain embodiments, the treatment modality is selected from the group consisting of surgical resection, chemotherapy, radiation, immunotherapy, and a combination thereof.

### 7.2.1. ATAC-QPCR Array Methods for Determining a Prognostic Score

Described are methods for determining the prognostic score of a subject using ATAC-QPCR arrays. The prognostic score can be used to assess, determine or predict the subject's responsiveness to one or more treatment modalities, disease-free survival score, and/or likelihood of cancer recurrence for pancreatic cancer and/or other cancers.

In one aspect, described are methods for determining a prognostic score indicative of a subject's responsiveness to one or more treatment modalities or indicative of a duration of disease-free survival, the method comprising: (a) contacting a biological sample obtained from the subject with a transposase complex to produce a population of tagged DNA fragments representing accessible chromatin regions of intact nuclei or intact nucleosome structure, wherein the biological sample comprises pancreatic ductal adenocarcinoma cells having morphologically intact nuclei or intact nucleosomes; (b) hybridizing a set of targeting oligonucleotide probes to a specific region on the accessible chromatin regions to generate targeted accessible chromatin region fragments, wherein the targeting oligonucleotide probes specifically target differentially accessible chromatin regions, and wherein the targeting oligonucleotide probes comprise: (i) a first subset of oligonucleotide probes targeting accessible chromatin regions associated with a first phenotype, and (ii) a second subset of oligonucleotide probes targeting accessible chromatin regions associated with a second phenotype; (c) amplifying the targeted accessible chromatin region fragments obtained in (b) that are associated with the first phenotype and the second phenotype; and (d) determining a prognostic score based on epigenetic signature values of the biological sample, wherein the prognostic score is determined based on a differential of a first epigenetic signature value and a second epigenetic signature value, and wherein the prognostic score is indicative of the subject's responsiveness to the one or more treatment modalities or indicative of a duration of disease-free survival.

In one aspect, described are methods of treating a subject having, or suspected of having, pancreatic ductal adenocarcinoma with one or more treatment modalities, the method comprising: (a) receiving a prognostic score indicative of the subject's responsiveness to one or more treatment modalities or indicative of a duration of disease-free survival, wherein the prognostic score is determined based on epigenetic signature values of a biological sample from the subject that comprises pancreatic ductal adenocarcinoma cells having morphologically intact nuclei or intact nucleosomes, the biological sample having been contacted with a transposase complex to produce a population of tagged DNA fragments representing accessible chromatin regions of the intact nuclei or intact nucleosome structure, wherein a set of targeting oligonucleotide probes were hybridized to a specific region on the accessible chromatin regions to generate targeted accessible chromatin region fragments, wherein the targeting oligonucleotide probes specifically target differentially accessible chromatin regions, wherein the prognostic score is determined based on a differential of a first epigenetic signature value and a second epigenetic signature value; and (b) treating the subject with the one or more treatment modalities based on the prognostic score.

The method can comprise collecting a biological sample from a subject having been diagnosed with a cancer (e.g., pancreatic cancer) or is at risk of having a cancer. In some embodiments, the biological sample is a tumor biopsy or surgically resected tumor specimen. In some embodiments, the biological sample comprises shredded cells from the subject such as epithelial cells or the subject's excreta (e.g., feces, saliva, sweat, earwax, mucus, urine). The biological sample can be collected by a physician, a surgeon, a healthcare provider, a laboratory technician or scientist. The biological sample may comprise pancreatic ductal adenocarcinoma cells. In some embodiments, the biological sample comprises treatment-naïve malignant cells obtained from a subject. In some embodiments, the biological sample comprises malignant cells from a subject who has been treated with one or more cancer therapies. In some embodiments, the biological sample is enriched for tumor cells. The enrichment can be achieved by contacting the biological sample with an agent (e.g., antibody-conjugated magnetic beads, EpCAM-conjugated magnetic beads) to isolate tumor cells from non-tumor cells in the biological sample to enrich the sample for tumor cells. In some embodiments, the agent is an EpCAM-conjugated magnetic beads. In some embodiments, the agent is an EpCAM-conjugated non-magnetic beads such as one used in flow-cytometry assays or immunohistochemistry assays.

Morphologically intact nuclei or intact nucleosomes of the enriched tumor cells (e.g., pancreatic ductal adenocarcinoma cells) can be extracted from the biological sample. The extracted intact nuclei can be used to generate an ATAC-library using enzymatic-based or non-enzymatic-based fragmentation of the genomic DNA into fragments. In some embodiments, fragmenting and tagging the accessible chromatin regions (ACRs) comprise using enzymatic tagementation such as using Tn transposase-based or restriction enzyme-based tagmentation. In some embodiments, fragmenting and tagging ACRs comprising using non-enzymatic tagmentation such as sonication, microfluidic shearing, or chemical cleavage (e.g., hydrogen peroxide, potassium permanganate). Example 2 in Section 8 illustrates an enzyme-based tagmentation method for generating ATAC libraries suitable for using the ATAC-qPCR array assay described herein. The ATAC-library can be used as the template for the ATAC-qPCR array assay.

The method may further comprise attaching a detectable label to the tagged DNA fragments to produce labeled fragments; and/or contacting the detectable labeled fragments to the set of targeting oligonucleotide probes. The method may further comprise quantifying the amount of amplified targeted accessible chromatin region fragments to obtain the first epigenetic signature value and the second epigenetic value. In some embodiments, the amplified targeted accessible chromatin region fragments are quantified by polymerase chain reaction (e.g., RT-PCR, qPCR), fluorometry, and/or gel electrophoresis. In some embodiments, quantification of the amplified targeted accessible chromatin region fragments does not involve sequencing the fragments.

In some embodiments, the set of targeting oligonucleotide probes are selected from any pair of oligonucleotide probes in Table 4. In some embodiments, the set of targeting oligonucleotide probes comprises at least 1, 2, 3, 4, 5, 6, 7, 8, 9, 10, 11, 12, 13, 14, 15, 16, 17, 18, 19, 20, or more pairs of oligonucleotide probes. In some embodiments, the set of targeting oligonucleotide probes are selected from any pair of oligonucleotide probes in Table 4. In some embodiments, at least 3 pairs of oligonucleotide probes are selected. In some embodiments, at least 8 pairs of oligonucleotide probes are selected. In some embodiments, at least 12 pairs of oligonucleotide probes are selected.

In some embodiments, the set of targeting oligonucleotide probes comprises no more than 2, 3, 4, 5, 6, 7, 8, 9, 10, 11, 12, 13, 14, 15, 16, 17, 18, 19, or 20 pairs of oligonucleotide probes. In some embodiments, the set of targeting oligonucleotide probes comprises no more than 3 pairs of oligonucleotide probes. In some embodiments, the set of targeting oligonucleotide probes comprises no more than 8 pairs of oligonucleotide probes. In some embodiments, the set of targeting oligonucleotide probes comprises no more than 12 pairs of oligonucleotide probes.

In some embodiments, the set of targeting oligonucleotide probes required for effectively determining the prognostic score is less than 100, 90, 80, 70, 60, 50, 40, 30, 20, or 10. In some embodiments, the set of targeting oligonucleotide probes required for effectively determining the prognostic score is about 12.

In some embodiments, the set of targeting oligonucleotide probes required for effectively determining good prognosis or poor prognosis is less than 100, 90, 80, 70, 60, 50, 40, 30, 20, or 10. In some embodiments, the set of targeting oligonucleotide probes required for effectively determining good prognosis is about 3. In some embodiments, the set of targeting oligonucleotide probes required for effectively determining poor prognosis is about 8.

In some embodiments, the pair of targeting oligonucleotide probes comprise a nucleic acid sequence having a sequence of any one of SEQ ID NOs: 1-30. In some embodiments, the pair of targeting oligonucleotide probes comprise a nucleic acid sequence having a sequence having at least 10%, at least 20%, at least 30%, at least 40%, at least 50%, at least 60%, at least 70%, at least 80%, at least 90%, or at least 99% identify to any one of SEQ ID NOs: 1-30.

In some embodiments, the set of targeting oligonucleotide probes comprises at least one set of probes targeting ARHGEF10, CLDN23, C12orf36, or SPATA4. In some embodiments, the set of targeting oligonucleotide probes comprises any two sets of probes targeting ARHGEF10, CLDN23, C12orf36, or SPATA4. In some embodiments, the set of targeting oligonucleotide probes comprises any three sets of probes targeting ARHGEF10, CLDN23, C12orf36, or SPATA4. In some embodiments, the set of targeting oligonucleotide probes comprises probes targeting ARHGEF10, CLDN23, C12orf36, SPATA4, or combinations thereof. In some embodiments, the set of targeting oligonucleotide probes comprises a set of probes targeting ARHGEF10, CLDN23, C12orf36, or SPATA4.

In some embodiments, the set of targeting oligonucleotide probes comprises at least one set of probes targeting MAP2K2, PPAP2B, CNBP, FIG. 4, TTC19, PAPL, RN7SL300P, or ITGAV. In some embodiments, the set of targeting oligonucleotide probes comprises at least two sets of probes targeting MAP2K2, PPAP2B, CNBP, FIG. 4, TTC19, PAPL, RN7SL300P, or ITGAV. In some embodiments, the set of targeting oligonucleotide probes comprises at least three sets of probes targeting MAP2K2, PPAP2B, CNBP, FIG. 4, TTC19, PAPL, RN7SL300P, or ITGAV. In some embodiments, the set of targeting oligonucleotide probes comprises at least 4, 5, 6, 7, or 8 sets of probes targeting MAP2K2, PPAP2B, CNBP, FIG. 4, TTC19, PAPL, RN7SL300P, or ITGAV. In some embodiments, the set of targeting oligonucleotide probes comprises probes targeting MAP2K2, PPAP2B, CNBP, FIG. 4, TTC19, PAPL, RN7SL300P, ITGAV, or combinations thereof. In some embodiments, the set of targeting oligonucleotide probes comprises a probe targeting MAP2K2, PPAP2B, CNBP, FIG. 4, TTC19, PAPL, RN7SL300P, or ITGAV.

In some embodiments, the set of targeting oligonucleotide probes comprises at least one set of probes targeting GTF3C6, LINC01703, C1orf131, or XRCC2. In some embodiments, the set of targeting oligonucleotide probes comprises at least two sets of probes targeting GTF3C6, LINC01703, C1orf131, or XRCC2. In some embodiments, the set of targeting oligonucleotide probes comprises at least three sets of probes targeting GTF3C6, LINC01703, C1orf131, or XRCC2. In some embodiments, the set of targeting oligonucleotide probes comprises probes targeting GTF3C6, LINC01703, C1orf131, XRCC2, or combinations thereof. In some embodiments, the set of targeting oligonucleotide probes comprises a set of probes targeting GTF3C6, LINC01703, C1orf131, or XRCC2.

In some embodiments, the set of targeting oligonucleotide probes comprises at least one set of probes targeting ARHGEF10, CLDN23, C12orf36, and SPATA4, at least one of MAP2K2, PPAP2B, CNBP, FIG. 4, TTC19, PAPL, or RN7SL300P, and at least one set of probes targeting GTF3C6, LINC01703, C1orf131, or XRCC2. In some embodiments, the set of targeting oligonucleotide probes comprises at least two sets of probes targeting ARHGEF10, CLDN23, C12orf36, and SPATA4, at least two of MAP2K2, PPAP2B, CNBP, FIG. 4, TTC19, PAPL, or RN7SL300P, and at least two sets of probes targeting GTF3C6, LINC01703, C1orf131, or XRCC2.

In some embodiments, the set of targeting oligonucleotide probes comprises at least one set of probes targeting ARHGEF10, CLDN23, C12orf36, SPATA4, MAP2K2, PPAP2B, CNBP, FIG. 4, TTC19, PAPL, RN7SL300P, GTF3C6, LINC01703, C1orf131, XRCC2, or combinations thereof. In some embodiments, the set of targeting oligonucleotide probes comprises any one set of probes targeting ARHGEF10, CLDN23, C12orf36, SPATA4, MAP2K2, PPAP2B, CNBP, FIG. 4, TTC19, PAPL, RN7SL300P, GTF3C6, LINC01703, C1orf131, XRCC2. In some embodiments, the set of targeting oligonucleotide probes comprises a set of probes targeting ARHGEF10, CLDN23, C12orf36, SPATA4, MAP2K2, PPAP2B, CNBP, FIG. 4, TTC19, PAPL, RN7SL300P, GTF3C6, LINC01703, C1orf131, XRCC2.

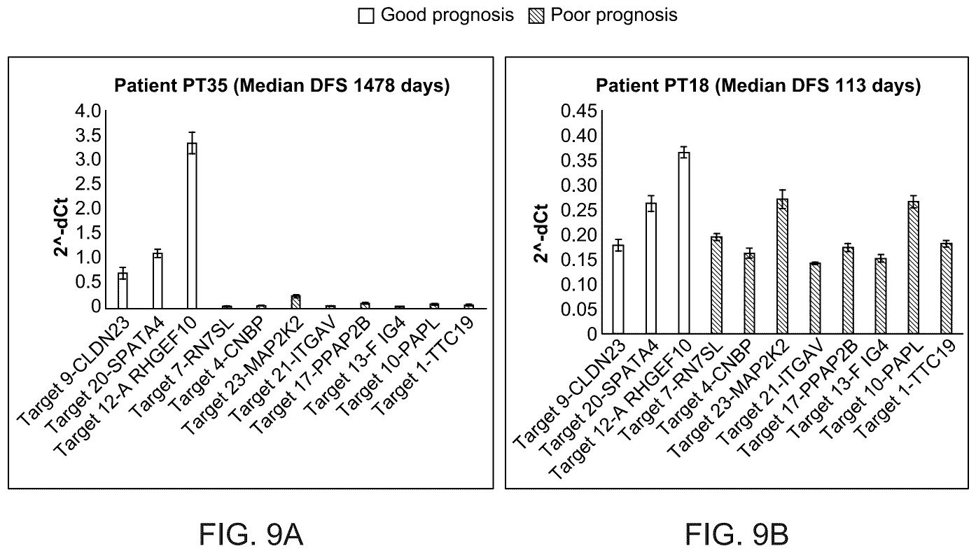

In some embodiments, the accessible chromatin regions are selected from any one of the accessible chromatin regions in FIG. 9A, FIG. 9B, and/or Table 3. In some embodiments, the accessible chromatin regions are selected from any one of the accessible chromatin regions in Table 3. In some embodiments, at least 1, 2, 3, 4, 5, 6, 7, 8, 9, 10, 11, 12, 13, 14, 15, 16, 17, 18, 19, or 20 accessible chromatin regions are selected from any one of the accessible chromatin regions in FIG. 9A, FIG. 9B, and/or Table 3. In some embodiments, at least 3 accessible chromatin regions are selected from any one of the accessible chromatin regions in FIG. 9A, FIG. 9B, and/or Table 3. In some embodiments, at least 8 accessible chromatin regions are selected from any one of the accessible chromatin regions in FIG. 9A, FIG. 9B, and/or Table 3. In some embodiments, at least 12 accessible chromatin regions are selected from any one of the accessible chromatin regions in FIG. 9A, FIG. 9B, and/or Table 3.

In some embodiments, the method further comprising the step of comparing the first epigenetic signature value to the second epigenetic signature value to obtain a differential value.

In some embodiments, the first epigenetic signature value is the median value of a set of accessible chromatin regions indicating a poor prognosis. In some embodiments, the median value of a set of accessible chromatin regions indicating a poor prognosis is the median value of at least 3 replications of ATAC-qPCR array analyses (e.g., the median Ct value) of the accessible chromatin regions selected from at least 1, 2, 3, 4, 5, 6, 7, or 8 of RN7SL, CNBP, MAP2K2, ITGAV, PPAP2B, FIG. 4, PAPL, and TTC19. In some embodiments, the accessible chromatin regions are selected from RN7SL, CNBP, MAP2K2, ITGAV, PPAP2B, FIG. 4, PAPL, TTC19, and/or combinations thereof.

In some embodiments, the second epigenetic signature value is the median value of a set of accessible chromatin regions indicating a good prognosis. In some embodiments, the median value of a set of accessible chromatin regions indicating a good prognosis is the median value of at least 3 replications of ATAC-qPCR array analyses (e.g., the median Ct value) of the accessible chromatin region selected from at least 1, 2, 3, or 4 of CLDN23, SPATA4, ARHGEF-10, and C12orf36. In some embodiments, the accessible chromatin regions are selected from CLDN23, SPATA4, ARHGEF-10, C12orf36, and/or combinations thereof.

In some embodiments, the method further comprising normalizing the differential value with at least one of a positive control epigenetic signature value and one of a negative control epigenetic signature value to obtain the prognostic score. In some embodiments, the control epigenetic signature value is determined by obtaining the median value of a set of accessible chromatin regions indicating the median value of at least 3 replications of ATAC-qPCR array analyses (e.g., the median Ct value) of the accessible chromatin regions selected from at least 1, 2, 3, or 4 of GTF3C6, LINC01703, C1orf131, and XRCC2. In some embodiments, the accessible chromatin regions are selected from GTF3C6, LINC01703, C1orf131, XRCC2, and/or combinations thereof.

In some embodiments, the prognostic score is at least 0.6 for determining a subject has a good prognosis or responsiveness to one or more treatment modalities described herein. In some embodiments, the prognostic score is less than 0.6 for determining a subject has a poor prognosis or responsiveness to one or more treatment modalities described herein.

In some embodiments, the method further comprising detecting nuclear localization of at least one of a transcription factor selected from any one of the transcription factors in Tables 2A and 2B. In some embodiments, the method further comprising detecting nuclear localization of at least 2, 3, 4, 5, 6, 7, 8, 9, 10, 15, 20, 25, 30, 35, 40, or 50 transcription factors selected from any one of the transcription factors in Tables 2A and 2B.

In some embodiments, the transcription factors are selected from ZKSCAN1, EPAS1, RUNX2, ZNF410, MAFF, RREB1, NR3C2, SMAD1, RUNX1, ZNF32, ZSCAN4, HOXB1, POU3F1, ZBTB3, CLOCK, TCF15, GCM1, HINFP, CGBP, MYPOP, ZNF384, GMEB2, E2F5, AC012531.1, ZBTB7B, HOXC9, HNF4G, CREB1, ATF2, E2F2, SP3, ARID5A, ZFP161, OTP, PBX3, ZBTB33, ONECUT3, ONECUT3, DLX2, HNF4A, PRRX1, TCFL5, HOXB7, IRF6, GRHL1, FOXD2, ISL1, MLL, GATA2, GATA1, HMBOX1, NRF1, ZFHX3, ONECUT1, TET1, E2F3, DNMT1, CTCFL, CTCF, HNF1B, and HNF1A.

In some embodiments, the transcription factors are selected from at least 2, 3, 4, 5, 6, 7, 8, 9, or 10 of transcription factors ZKSCAN1, EPAS1, RUNX2, ZNF410, MAFF, RREB1, NR3C2, SMAD1, RUNX1, ZNF32, ZSCAN4, HOXB1, POU3F1, ZBTB3, CLOCK, TCF15, GCM1, HINFP, CGBP, MYPOP, ZNF384, GMEB2, E2F5, AC012531.1, ZBTB7B, HOXC9, HNF4G, CREB1, ATF2, E2F2, SP3, ARID5A, ZFP161, OTP, PBX3, ZBTB33, ONECUT3, ONECUT3, DLX2, HNF4A, PRRX1, TCFL5, HOXB7, IRF6, GRHL1, FOXD2, ISL1, MLL, GATA2, GATA1, HMBOX1, NRF1, ZFHX3, ONECUT1, TET1, E2F3, DNMT1, CTCFL, CTCF, HNF1B, and HNF1A.

In some embodiments, the transcription factors are selected from ZKSCAN1A, HNF1B, or a combination thereof.

In some embodiments, the method further comprises predicting a long duration of disease-free survival when the second epigenetic signature value is significantly higher than the second epigenetic signature value and/or predicting a short duration of disease-free survival when the first epigenetic signature value is significantly higher than the second epigenetic value.

In some embodiments, the first epigenetic signature value indicates a first phenotype.

In some embodiments, the first phenotype is recurrence of a cancer within 6 months, or 1, 2, 3, 4, or 5 years of surgical resection. In some embodiments, the first phenotype is recurrence of a cancer within one year of surgical resection.

In some embodiments, the first phenotype is recurrence of a cancer within one year of surgical resection. In some embodiments, the first phenotype is recurrence of a cancer within 11 months, 10 months, 9 months, 8 months, 7 months, 6 months, 5 months, 4 months, 3 months, 2 months or 1 month of surgical resection.

In some embodiments, the first phenotype is having a median disease-free survival of less than 5, 4, 2, 3, 1 or less years. In some embodiments, the second phenotype is having a median disease-free survival of less than 12, 11, 10, 9, 8, 7, 6, 5, 4, 3, 2, 1 or less months. In some embodiments, the second phenotype is having a median disease-free survival of less than 350, 300, 250, 200, 150, 100, 50, 10, or less days. In some embodiments, the second phenotype is having a median disease-free survival of less than 50 days.

In some embodiments, the first phenotype is having a median disease-free survival of between 1 to 350 days, between 50-300 days, between 10-100 days, or between 40 to 250 days.

In some embodiments, the second epigenetic signature value indicates a second phenotype.

In some embodiments, the second phenotype is non-recurrence of a cancer within 6 months, or 1, 2, 3, 4, or 5 years of surgical resection. In some embodiments, the second phenotype is non-recurrence of a cancer within one year of surgical resection.

In some embodiments, the second phenotype is non-recurrence of a cancer within one year of surgical resection. In some embodiments, the second phenotype is non-recurrence of a cancer within 11 months, 10 months, 9 months, 8 months, 7 months, 6 months, 5 months, 4 months, 3 months, 2 months or 1 month of surgical resection.

In some embodiments, the second phenotype is having a median disease-free survival (DFS) of at least of 1, 2, 3, 4, 5, 6, 7, 8, 9, 10, or more years.

In some embodiments, the second phenotype is having a median disease-free survival (DFS) of at least 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, or more days.

In some embodiments, the second phenotype is having a median disease-free survival (DFS) of between 50 to 1500 days, between 100 to 1000 days, between 300 to 800 days, between 200 to 500 days, or between 400 to 600 days. In some embodiments, the first phenotype is having a median disease-free survival of at least 350 days.

In some embodiments, the first phenotype is non-responder to a cancer therapy. In some embodiments, the second phenotype is responder to a cancer therapy. In some embodiments, the cancer therapy is selected from chemotherapy, immunotherapy, radiation, or combinations thereof.

In some embodiments, the biological sample comprises treatment-naïve malignant cells obtained from a subject. In some embodiments, the subject is a treatment naïve patient who has not received the one or more treatment modalities for a cancer such as pancreatic or other cancers. In some embodiments, the subject has not been diagnosed with a cancer such as pancreatic or other cancers.

In some embodiments, the subject is under treatment or has been treated with the one or more treatment modalities for a cancer such as pancreatic or other cancers.

In some embodiments, the one or more treatment modalities are selected from resecting cancerous tissue, neo-adjuvant chemotherapy, adjuvant chemotherapy, immunotherapy, an epigenetic drug, or combinations thereof. In some embodiments, the epigenetic drug is selected from DNMT inhibitor, an HDAC inhibitor, an EZH2 inhibitor, or combinations thereof. In some embodiments, the DNMT inhibitor is azacytidine or decitabine. In some embodiments, the HDAC inhibitor is vorinostat or romidepsin.

In some embodiments, the neo-adjuvant chemotherapy and/or adjuvant chemotherapy is selected from gemcitabine, nab-paclitaxel, fluorouracil (5-FU), irinotecan, oxaliplatin, leucovorin, capecitabine, cisplatin, or combinations thereof.

In some embodiments, the biological sample is selected from a tumor biopsy or surgically resected tumor specimen.

In some embodiments, the method does not include sequencing the tagged fragments or amplicons thereof. In some embodiments, obtaining the targeted accessible chromatin region fragments does not include the step of sequencing the tagged fragments or amplicons thereof.

In some embodiments, the amplified targeted accessible chromatin fragments comprise a mean size of about 50 bp to about 1100 bp, about 100 bp to about 350 bp, about 150 bp to about 800 bp, about 80 bp to about 150 bp, or about 150 bp to about 400 bp. In some embodiments, the amplified targeted accessible chromatin fragments comprise a mean size of 80 bp, about 100 bp, about 120 bp, about 150 bp, about 180 bp, about 200 bp, about 250 bp, about 300 bp, about 400 bp, about 500 bp, about 600 bp, about 700 bp, about 800 bp, about 900 bp, about 1000 bp, about 1100 bp, or longer. In some embodiments, the amplified targeted accessible chromatin fragments comprise a mean size of about 120 bp.

In accordance with any one of the embodiments, the set of targeting oligonucleotide probes required for effectively determining the prognostic score is less than 100, 90, 80, 70, 60, 50, 40, 30, 20, or 10.

In some embodiments, the set of targeting oligonucleotide probes required for effectively determining the prognostic score is about 12.

In some embodiments, the set of targeting oligonucleotide probes required for effectively determining good prognosis or poor prognosis is less than 100, 90, 80, 70, 60, 50, 40, 30, 20, or 10.

In some embodiments, the set of targeting oligonucleotide probes required for effectively determining good prognosis is about 3.

In some embodiments, the set of targeting oligonucleotide probes required for effectively determining poor prognosis is about 8.

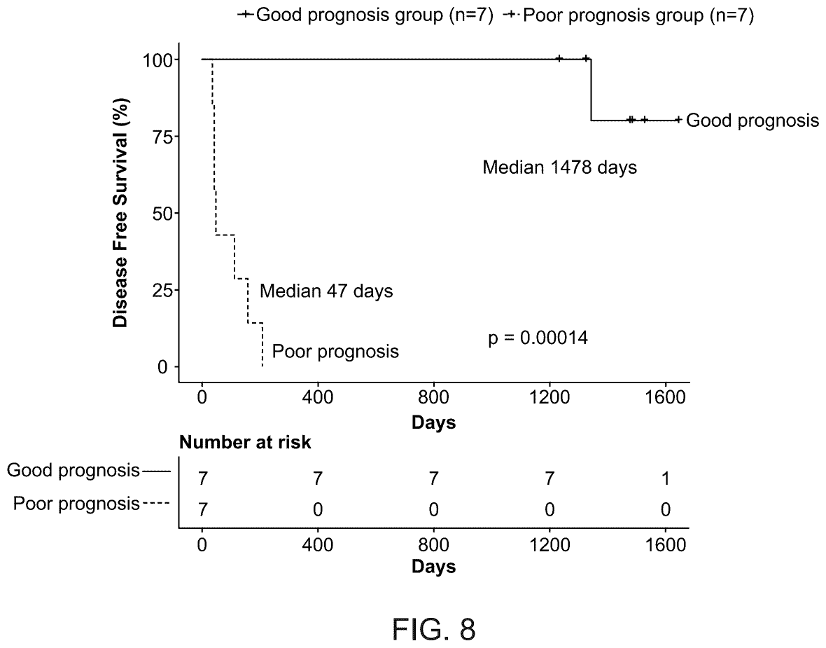

In some embodiments, the method monitors patients with pancreatic cancer who had undergone upfront surgery followed by Gemcitabine/Nab-Paclitaxel adjuvant chemotherapy. These patients are monitored for a median follow-up time of at least 1, 2, 3, 4, 5 or more years post-surgery. FIG. 8 shows the Kaplan-Meier graph showing patients with good prognosis and poor prognosis.

In some embodiments, the patients are monitored for 4.15 years post-surgery. Patients who show a median disease-free survival (DFS) of at least 100, 200, 300, 400, 500, 600, 700, 800, 900, 1000, 1200, 1400, 1600, 1800, 2000, or more days are determined as having good prognosis. In some embodiments, patients having good prognosis have a median DFS of between about 1000 days to 1800 days, for example, ranging from 1234 days to 1645 days. In some embodiments, patients having a good prognosis have a median DFS of 1478 days.

In some embodiments, at least 10%, at least 20%, at least 30%, at least 40%, at least 50%, at least 60%, at least 70%, at least 80%, at least 90%, at least 99%, or more of patients having good prognosis have a median DFS of more than 400 days, 800 days, 1200 days, 1600 days, or more. In some embodiments, at least 10%, at least 20%, at least 30%, at least 40%, at least 50%, at least 60%, at least 70%, at least 80%, at least 90%, at least 99%, or more of patients having a good prognosis have a median DFS of between 400 to 1600 days, 1200 to 1500 days, or 600 to 1400 days. In some embodiments, at least 75% of patients having a good prognosis have a median DFS of about 1400 days.

In some embodiments, patients predicted with a good prognosis have a median DFS of at least 400 days, 800 days, 1200 days, 1600 days, or more. In some embodiments, patients predicted with a good prognosis have a median DFS of between 400 to 1600 days, 1200 to 1500 days, or 600 to 1400 days. In some embodiments, patients predicted with a good prognosis have a median DFS of about 1478 days.

Patients who show a median disease-free survival (DFS) of less than 500, 400, 300, 200, 100, 50, 20, or 10 days are determined as having a poor prognosis. In some embodiments, patients having poor prognosis have a median DFS of between 30 days to 300 days, for example, ranging from 36 to 207 days. In some embodiments, patients having poor prognosis have a median DFS of 47 days.

In some embodiments, at least 10%, at least 20%, at least 30%, at least 40%, at least 50%, at least 60%, at least 70%, at least 80%, at least 90%, at least 99%, or more of patients having poor prognosis have a median DFS of less than 400 days, 200 days, 100 days, 50 days, or less. In some embodiments, at least 10%, at least 20%, at least 30%, at least 40%, at least 50%, at least 60%, at least 70%, at least 80%, at least 90%, at least 99%, or more of patients having a poor prognosis have a median DFS of between 10 to 400 days, 50 to 200 days, or 10 to 50 days. In some embodiments, at least 25% of patients having a poor prognosis have a median DFS of about 47 days.

In some embodiments, patients predicted with a poor prognosis have a median DFS of 400 days, 200 days, 100 days, 50 days, or less. In some embodiments, patients predicted with a poor prognosis have a median DFS of between 10 to 400 days, 50 to 200 days, or 10 to 50 days. In some embodiments, patients predicted with a poor prognosis have a median DFS of about 47 days

The relative accessibility of the differentially accessible chromatin regions (the solid line indicates good prognosis and the broken line indicates poor prognosis, normalized with the control) is estimated by 2\hat{ }-dCt method in each patient by qPCR analyses of the individual ATAC-libraries prepared from them. The dCt values for each target region can be calculated as below:

dCt=Ct values of the differential (blue or red) regions−Geometric mean Ct values of the control (green) regions

FIGS. 9A and 9B show the results of ATAC-qPCR analysis of 11 target regions using 4 control regions for normalization. The relative quantitation of blue and red regions in each individual patient were shown by bar plot, where the open bars represented the blue (good prognosis) target regions and the striped bars represented the red (poor prognosis) target regions.

The Prognostic scores can be calculated using the ATAC-qPCR analytical methods as displayed in the formula below:

Prognostic score (PS) by ATAC-qPCR array=2\hat{ }^{-dCt} of blue regions−2\hat{ }^{-dCt} of red regions

As an example, FIG. 9A shows Patient PT35 having a median DFS of 1478 days. PT35 has high 2\hat{ }⁻dCt values of good prognosis epigenetic signature values of accessible chromatin regions (e.g., CLDN23, SPATA4, and ARHGEF10), which have 2\hat{ }⁻dCt values of between about 0.6 to about 3.5. PT35 has low 2\hat{ }⁻dCt values of poor prognosis epigenetic signature values of accessible chromatin regions (e.g., RN7SL, CNBP, MAP2K2, ITGAV, PPAP2B, FIG. 4, PAPL, and TTC19), which have 2\hat{ }⁻dCt values of between about 0.01 to about 0.2. For example, the normalized differential values of CLND23 and MAP2K2 is about 0.6 to about 0.2, indicating a prognostic score (PS) of 0.4. The normalized differential value of SPATA4 to MAP2K2 is about 1 to about 0.2, indicating a PS of 0.8. The normalized differential value of ARHGEF10 and MAP2K2 is about 3.2 to about 0.2, indicating a PS of 2.8. Based on the formula, PT35 has a prognostic score of 0.8 and 2.8, which is higher than 0.6, when comparing the 2\hat{ }dCt values of a good prognosis epigenetic signature value (e.g., SPATA4, and ARHGEF10) with a poor prognosis score (e.g., MAP2K2). As another example, an average of the normalized differential values of good prognosis epigenetic signature values of accessible chromatin regions including CLDN23, SPATA4, and ARHGEF10 is compared to an average of the normalized differential values of poor prognosis epigenetic signature values of accessible chromatin regions including RN7SL, CNBP, MAP2K2, ITGAV, PPAP2B, FIG. 4, PAPL, and TTC19 to determine a prognostic score. For illustration purposes, the average of the normalized differential values of good prognosis epigenetic signature values of CLDN23, SPATA4, and ARHGEF10 is about 1.67, and the average of the normalized differential values of good prognosis epigenetic signature values of RN7SL, CNBP, MAP2K2, ITGAV, PPAP2B, FIG. 4, PAPL, and TTC19 is about 0.12, giving a prognostic score of 1.55, which is higher than 0.6.

FIG. 9B shows Patient PT18 having a median DFS of 113. PT18 has 2\hat{ }⁻dCt values between about 0.15 to about 0.4 of good prognosis epigenetic signature values of accessible chromatin regions (e.g., CLDN23, SPATA4, and ARHGEF10), and has 2\hat{ }⁻dCt values between about 0.15 to about 0.3 of poor prognosis epigenetic signature values of accessible chromatin regions (e.g., RN7SL, CNBP, MAP2K2, ITGAV, PPAP2B, FIG. 4, PAPL, and TTC19). For example, the normalized differential value of CLND23 and MAP2K2 is about 0.18 to about 0.28, indicating a prognostic score (PS) of −0.1. The normalized differential value of SPATA4 and MAP2K2 is about 0.25 to about 0.28, indicating a prognostic score (PS) of −0.03. The normalized differential value of ARHGEF10 and MAP2K2 is about 0.38 to about 0.28, indicating a prognostic score (PS) of 0.1. Based on the formula, PT18 has a prognostic score of −0.1, −0.03, and 0.1, which is lower than 0.6, when comparing the 2\hat{ }dCt values of a good prognosis epigenetic signature value (e.g., CLDN23, SPATA4, and ARHGEF10) with a poor prognosis score (e.g., MAP2K2). As another example, an average of the normalized differential values of good prognosis epigenetic signature values of accessible chromatin regions including CLDN23, SPATA4, and ARHGEF10 is compared to an average of the normalized differential values of poor prognosis epigenetic signature values of accessible chromatin regions including RN7SL, CNBP, MAP2K2, ITGAV, PPAP2B, FIG. 4, PAPL, and TTC19 to determine a prognostic score. For illustration purposes, the average of the normalized differential values of good prognosis epigenetic signature values of CLDN23, SPATA4, and ARHGEF10 is about 0.27, and the average of the normalized differential values of good prognosis epigenetic signature values of RN7SL, CNBP, MAP2K2, ITGAV, PPAP2B, FIG. 4, PAPL, and TTC19 is about 0.21, giving a prognostic score of 0.06, which is lower than 0.6.

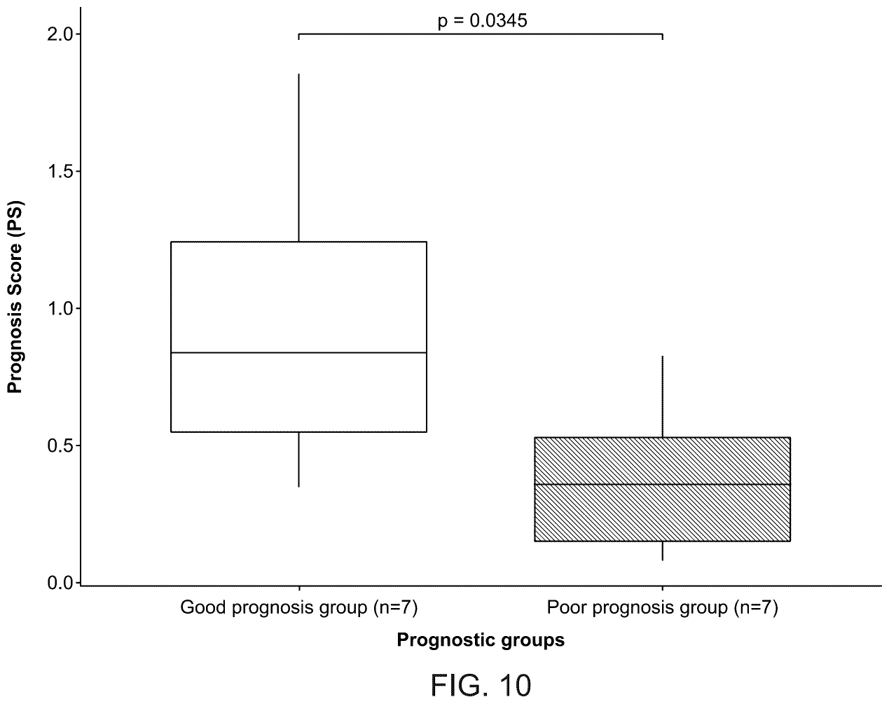

Using the formula described above, the individualized PS for each patient can be calculated and the PS of good and poor prognosis groups can be analyzed. The box plot as displayed in FIG. 10 demonstrated a statistically significant difference (t test p=0.034) of PS between the 7 good and 7 poor prognosis patients. Open bar represents good prognosis. Stripped bar represents poor prognosis.

In some embodiments, a prognostic score of between 0.3 to 2, between 0.5 to 1.5, or between 0.6 to 1.3 provides the prediction for a good prognosis. In some embodiments, a prognostic score of about 0.3, 0.6, 0.9, 1.2, 1.5, 1.8, or above provides the prediction for a good prognosis. In some embodiments, a prognostic score of above 0.6 provides the prediction for a good prognosis.

In some embodiments, a prognostic score of between 0.1 to 1, between 0.2 to 0.6, or between 0.4 to 0.8 provides the prediction for a poor prognosis. In some embodiments, a prognostic score of about 1, 0.9, 0.8, 0.7, 0.6, 0.5, 0.4, 0.3, 0.2, 0.1 or less provides the prediction for a poor prognosis. In some embodiments, a prognostic score of less than 0.6 provides the prediction for a poor prognosis.

The method can successfully determine a set of targeting oligonucleotide probes required for effectively determining a reliable prognostic score. In some embodiments, the set of targeting oligonucleotide probes required can be less than 100, or as few as 12 probes. The set of targeting oligonucleotide probes required for effectively determining good prognosis can be as few as 3 probes, and the set of targeting oligonucleotide probes required for effectively determining poor prognosis can be as few as 8 probes. The method using ATAC-qPCR array is a robust, reliable, and cost-effective approach for determining prognostic score of a patient having, or at risk of developing pancreatic cancer. Such information is critical for the selection of treatment modalities, which can enhance the subject's likelihood of disease-free survival.

### 7.3 Diagnosis, Prognosis, and Treatment of Cancer

In one aspect, the present disclosure provides a diagnostic or prognostic method. In certain embodiments, the diagnostic or prognostic method may distinguish between treatment-resistant and treatment-sensitive cancers. In certain embodiments, the diagnostic or prognostic method may distinguish between rapidly recurrent and non-recurrent tumors. In some such embodiments, the tumors are pancreatic tumors, such as pancreatic ductal adenocarcinoma.

In certain embodiments, the diagnostic or prognostic method comprises determining a epigenetic landscape from a biological sample obtained from a patient, wherein the epigenetic landscape comprises at least two, alternatively at least five, at least ten, at least twenty, at least thirty, at least forty, at least fifty, at least one hundred, at least two hundred, at least three hundred, at least four hundred, at least five hundred, at least six hundred, at least seven hundred, at least eight hundred, at least nine hundred, or at least one thousand chromatin regions selected from the list of chromatin regions in Table 1 and/or Table 2; and providing a diagnosis or prognosis based on the determination. In typical embodiments, the diagnostic or prognostic method comprises determining a epigenetic landscape comprising no more than 100 open chromatin regions in Table 1 and/or Table 2.

In one aspect, the present disclosure provides a method for treating a disease or condition such as cancer, particularly pancreatic cancer. In certain embodiments, the method comprises performing surgical resection to remove a pancreatic ductal adenocarcinoma from a patient, wherein prior to said resection a biological sample from the patient has been tested to determine an epigenetic landscape of the biological sample. In some such embodiments, the epigenetic landscape comprises a plurality of pre-selected differentially accessible chromatin regions and the plurality of pre-selected differentially accessible chromatin regions comprise at least two, alternatively at least five, at least ten, at least twenty, at least thirty, at least forty, at least fifty, at least one hundred, at least two hundred, at least three hundred, at least four hundred, at least five hundred, at least six hundred, at least seven hundred, at least eight hundred, at least nine hundred, or at least one thousand chromatin regions selected from the list of chromatin regions in Table 1 and/or Table 2, which provides a signature of >1000 loci that were differentially accessible between recurrent (disease free survival (DFS)<1 year) and non-recurrent patients (DFS>1 year). In typical embodiments, the diagnostic or prognostic method comprises determining a epigenetic landscape comprising no more than 100 differentially accessible open chromatin regions in Table 1 and/or Table 2. In certain embodiments, the method comprises performing surgical resection to remove a pancreatic ductal adenocarcinoma from a patient, wherein prior to said resection a biological sample from the patient has been tested to determine nuclear localization of one or more transcription factors.

In one aspect, the present disclosure provides a method for treating a disease or condition such as cancer, particularly pancreatic cancer. In certain embodiments, the method comprises administering a drug to the patient, wherein prior to administering the drug, a biological sample from the patent has been tested to determine an epigenetic landscape of the biological sample.

In certain embodiments, the patient is identified as likely being a non-responder to a treatment modality. In some such embodiments, the treatment modality is surgical resection with or without adjuvant chemotherapy. In certain embodiments, the patient is identified as having a tumor likely to recur within one year following surgical resection and adjuvant chemotherapy. In some such embodiments, the tumor is a pancreatic ductal adenocarcinoma.

In certain embodiments, the epigenetic landscape comprises a plurality of pre-selected differentially accessible chromatin regions and the plurality of pre-selected differentially accessible chromatin regions comprise at least two, alternatively at least five, at least ten, at least twenty, at least thirty, at least forty, at least fifty, at least one hundred, at least two hundred, at least three hundred, at least four hundred, at least five hundred, at least six hundred, at least seven hundred, at least eight hundred, at least nine hundred, or at least one thousand chromatin regions selected from the list of chromatin regions in Table 1, which provides a signature of >1000 loci that were differentially accessible between recurrent (disease-free survival (DFS)<1 year) and non-recurrent patients (DFS>1 year). In typical embodiments, the epigenetic landscape comprises no more than 100 pre-selected differentially accessible chromatin regions in Table 1 and/or Table 2.

In certain embodiments, the method comprises administering the epigenetic drug to the patient, wherein prior to said administration a biological sample from the patient has been tested to determine nuclear localization of one or more transcription factors.

In one aspect, the present disclosure provides a method for treating cancer in a patient in need thereof. In certain embodiments, the method comprises (a) assessing if the patient is likely to be a responder or a non-responder to a first treatment modality by determining or having determined an epigenetic landscape of a biological sample obtained from the cancer patient; and (b) treating the cancer patient with a second treatment modality if the patient is determined to be a likely non-responder to the first treatment modality.

### 7.3.1. Prognosis of Cancer

Described herein are methods for determining a prognosis of a cancer. The method can comprise determining and/or providing a prognostic score indicative of a subject's responsiveness to one or more treatment modalities or indicative of a duration of disease-free survival as described in Section 7.2.1, and Examples 2, 5, 6, and 7. In some embodiments, the method further comprises determining or providing a first epigenetic signature value, a second epigenetic signature value, a good prognosis report, a poor prognosis report, a disease-free survival score, a report of a likelihood of cancer recurrence or non-recurrence, and/or a prediction of treatment modality responsiveness.

In some embodiments, prognosis of a cancer comprises determining a first epigenetic signature value and a second epigenetic signature value. The first epigenetic signature value indicates a first phenotype, which is a poor prognosis of the cancer. The second epigenetic signature value indicates a second phenotype, which is a good prognosis of the cancer.

In some embodiments, prognosis of the cancer comprises using the first and second epigenetic signature values to determine the likelihood of recurrence or non-recurrence of a cancer. For example, a high first epigenetic signature value may indicate a higher likelihood of recurrence of the cancer within a year or less. A high second epigenetic signature value may indicate a lower likelihood of recurrence of the cancer within a year or less.

The differential of the first epigenetic signature value and the second epigenetic signature value can be normalized with a control epigenetic signature value to determine a prognostic score. For example, a prognostic score of less than 0.6 indicates recurrence of the cancer or poor responsiveness (e.g., non-responder) to one or more cancer treatment modalities. Conversely, a prognostic score of higher than 0.6 indicates non-recurrence of the cancer or good responsiveness (e.g., responder) to one or more cancer treatment modalities.

In some embodiments, the one or more treatment modalities are selected from resecting cancerous tissue, neo-adjuvant chemotherapy, adjuvant chemotherapy, immunotherapy, an epigenetic drug, or combinations thereof. In some embodiments, the epigenetic drug is selected from DNMT inhibitor, an HDAC inhibitor, an EZH2 inhibitor, or combinations thereof. In some embodiments, the DNMT inhibitor is azacytidine or decitabine. In some embodiments, the HDAC inhibitor is vorinostat or romidepsin. In some embodiments, the neo-adjuvant chemotherapy and/or adjuvant chemotherapy is selected from gemcitabine, nab-paclitaxel, fluorouracil (5-FU), irinotecan, oxaliplatin, leucovorin, capecitabine, cisplatin, or combinations thereof.

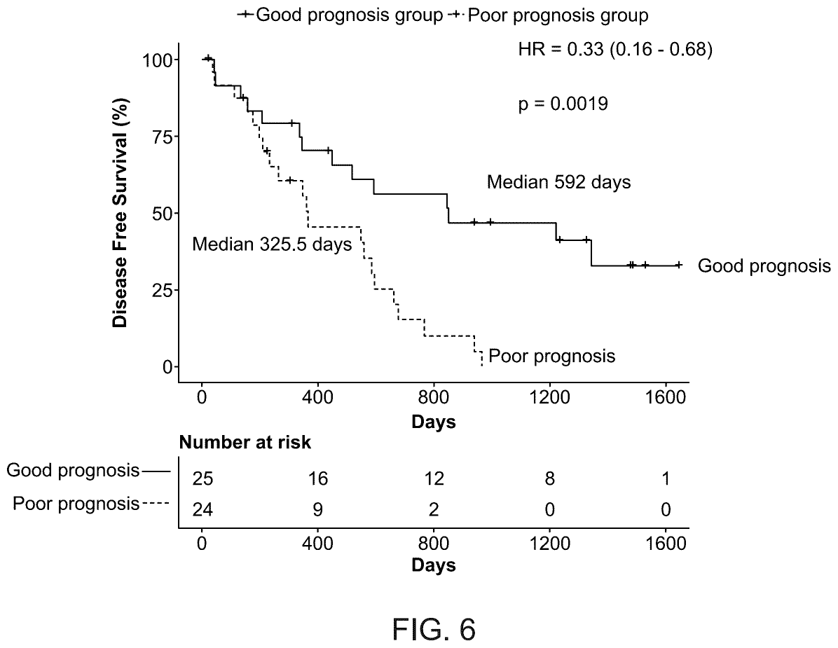

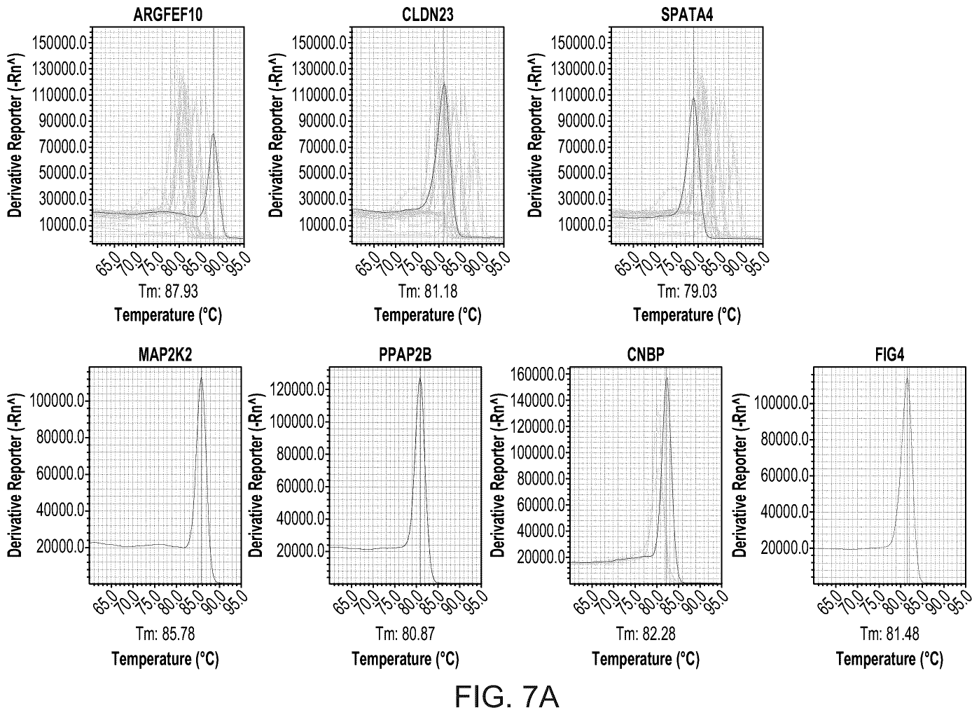

In some embodiments, the prognostic score is used to determine the disease-free survival (DFS) of the subject. For example, a subject having a prognostic score of higher than 0.6 may have a median DFS of at least of 1, 2, 3, 4, 5, 6, 7, 8, 9, 10, or more years, or at least 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, or more days. FIG. 6 shows subjects having a median DFS of 592 days are determined to have a good prognosis, and subjects having a median DFS of 325.5 days are determined to have a poor prognosis.

The prognostic score can be determined by assessing the differential values of good prognosis and poor prognosis by obtaining the epigenetic signature values of accessible chromatin regions as shown in FIGS. 9A and 9B, and/or Table 3. In some embodiments, the prognostic score can be determined by assessing the differential values of good prognosis and poor prognosis obtaining the epigenetic signature values of less than 12, 8, 4, or 3 accessible chromatin regions as shown in FIGS. 9A and 9B, and/or Table 3. Using the accessible chromatic regions in Table 3 to determine the prognostic score, FIG. 8A subjects having a median DFS of 1478 days are determined to have a good prognosis, and subjects having a median DFS of 47 days are determined to have a poor prognosis.

In some embodiments, the prognostic score is determined or provided by a physician, a surgeon, a healthcare provider, a laboratory technician or scientist, or a data analysist.

### 7.3.2. Treatment of Cancer

Described herein are methods for determining treatment modalities for a cancer such as pancreatic or other cancers. The method comprises determining and/or receiving a prognostic score indicative of a subject's responsiveness to one or more treatment modalities or indicative of a duration of disease-free survival and treating the subject with the one or more treatment modalities based on the prognostic score. In some embodiments, the prognostic score is determined using the ATAC-array qPCR assays described in Section 7.2.1, Section 7.3.1, and Examples 2, 5, 6, and 7. In some embodiments, the method further comprises determining or receiving a first epigenetic signature value, a second epigenetic signature value, a good prognosis report, a poor prognosis report, a disease-free survival score, a report of a likelihood of cancer recurrence or non-recurrence, and/or or prediction of treatment modality responsiveness.

A subject having a prognostic score higher than 0.6, a good prognosis report, indication of non-recurrence, or a high disease-free survival score may be prescribed or administered with one or more treatment modalities. Conversely, a subject having a prognostic score lower than 0.6, a poor prognosis report, indication or recurrence, or a low disease-free survival score may be advised to have no further treatment modalities.

In some embodiments, the one or more treatment modalities are selected from resecting cancerous tissue, neo-adjuvant chemotherapy, adjuvant chemotherapy, immunotherapy, an epigenetic drug, or combinations thereof. In some embodiments, the epigenetic drug is selected from DNMT inhibitor, an HDAC inhibitor, an EZH2 inhibitor, or combinations thereof. In some embodiments, the DNMT inhibitor is azacytidine or decitabine. In some embodiments, the HDAC inhibitor is vorinostat or romidepsin. In some embodiments, the neo-adjuvant chemotherapy and/or adjuvant chemotherapy is selected from gemcitabine, nab-paclitaxel, fluorouracil (5-FU), irinotecan, oxaliplatin, leucovorin, capecitabine, cisplatin, or combinations thereof.

In some embodiments, the treatment modalities are prescribed or administered by a physician, a surgeon, a healthcare provider, a laboratory technician or scientist, or a data analysist.

In some embodiments, the subject is a treatment naïve patient who has not received the one or more treatment modalities for a cancer such as pancreatic or other cancers. In some embodiments, the subject has not been diagnosed with a cancer such as pancreatic or other cancers. In some embodiments, the subject is under treatment or has been treated with the one or more treatment modalities for a cancer such as pancreatic or other cancers.

### 7.4 Kits

In one aspect, described are kits for determining an epigenetic landscape associated with a specific phenotypic trait of a biological sample, the kit comprising:
(d) an array configured to detect targeted accessible chromatin region fragments obtained from the biological sample, wherein the array comprises a panel of sets of targeting oligonucleotide probes specifically targeting differentially accessible chromatin regions;
(e) reagents; and
(f) instructions for amplifying and quantifying the targeted accessible chromatin region fragments to obtain a first epigenetic signature value and a second epigenetic value.

In one aspect, described are kits for determining an epigenetic landscape associated with a specific phenotypic trait of a biological sample, the kit comprising:
(d) one or more sets of targeting oligonucleotide probes for detect targeted accessible chromatin region fragments obtained from the biological sample, wherein the targeting oligonucleotide probes specifically target and amplify differentially accessible chromatin regions to generate one or more targeted accessible chromatin region fragments;
(e) reagents; and
(f) instructions for amplifying and quantifying the targeted accessible chromatin region fragments to obtain a first epigenetic signature value and a second epigenetic value.

In some embodiments, the targeting oligonucleotide probes are provided in a panel. In some embodiments, the panel of targeting oligonucleotide probes is arranged as described in FIG. 4A or 4B.

In some embodiments, the targeting oligonucleotide probes are provided in a 96-well plate. In some embodiments, the targeting oligonucleotide probes are provided in a 384-well plate. In some embodiments, the targeting oligonucleotide probes are provided in one or more individual containers, e.g., an Eppendorf tube.

In some embodiments, the biological sample comprises pancreatic ductal adenocarcinoma cells having morphologically intact nuclei or intact nucleosome (histone-DNA) structure.

In some embodiments, the kit further comprises reagents and instructions for obtaining the biological sample by contacting the morphologically intact nuclei or intact nucleosome (histone-DNA) structure to a transposase complex to produce a population of tagged DNA fragments representing the targeted accessible chromatin region fragments.

In some embodiments, the pair of targeting oligonucleotide probes comprise a nucleic acid sequence having a sequence of any one of SEQ ID NOs: 1-30. In some embodiments, the pair of targeting oligonucleotide probes comprise a nucleic acid sequence having a sequence having at least 10%, at least 20%, at least 30%, at least 40%, at least 50%, at least 60%, at least 70%, at least 80%, at least 90%, or at least 99% identify to any one of SEQ ID NOs: 1-30.

In some embodiments, the set of targeting oligonucleotide probes comprises at least 1, 2, 3, 4, 5, 6, 7, 8, 9, 10, 11, 12, 13, 14, 15, 16, 17, 18, 19, 20, or more pairs of oligonucleotide probes. In some embodiments, the set of targeting oligonucleotide probes are selected from any pair of oligonucleotide probes in Table 4. In some embodiments, at least 3 pairs of oligonucleotide probes are selected. In some embodiments, at least 8 pairs of oligonucleotide probes are selected. In some embodiments, at least 12 pairs of oligonucleotide probes are selected.

In some embodiments, the set of targeting oligonucleotide probes comprises no more than 2, 3, 4, 5, 6, 7, 8, 9, 10, 11, 12, 13, 14, 15, 16, 17, 18, 19, or 20 pairs of oligonucleotide probes. In some embodiments, the set of targeting oligonucleotide probes comprises no more than 3 pairs of oligonucleotide probes. In some embodiments, the set of targeting oligonucleotide probes comprises no more than 8 pairs of oligonucleotide probes. In some embodiments, the set of targeting oligonucleotide probes comprises no more than 12 pairs of oligonucleotide probes.

In some embodiments, the set of targeting oligonucleotide probes required for effectively determining the prognostic score is less than 100, 90, 80, 70, 60, 50, 40, 30, 20, or 10. In some embodiments, the set of targeting oligonucleotide probes required for effectively determining the prognostic score is about 12.

In some embodiments, the set of targeting oligonucleotide probes required for effectively determining good prognosis or poor prognosis is less than 100, 90, 80, 70, 60, 50, 40, 30, 20, or 10. In some embodiments, the set of targeting oligonucleotide probes required for effectively determining good prognosis is about 3. In some embodiments, the set of targeting oligonucleotide probes required for effectively determining poor prognosis is about 8.

In some embodiments, the set of targeting oligonucleotide probes comprises probes targeting at least one of ARHGEF10, CLDN23, C12orf36, or SPATA4. In some embodiments, the set of targeting oligonucleotide probes comprises probes targeting any two of ARHGEF10, CLDN23, C12orf36, or SPATA4. In some embodiments, the set of targeting oligonucleotide probes comprises probes targeting any three of ARHGEF10, CLDN23, C12orf36, or SPATA4. In some embodiments, the set of targeting oligonucleotide probes comprises probes targeting ARHGEF10, CLDN23, C12orf36, SPATA4, or combinations thereof. In some embodiments, the set of targeting oligonucleotide probes comprises probes targeting ARHGEF10, CLDN23, C12orf36, or SPATA4.

In some embodiments, the set of targeting oligonucleotide probes comprises probes targeting at least one of MAP2K2, PPAP2B, CNBP, FIG. 4, TTC19, PAPL, RN7SL300P, or ITGAV. In some embodiments, the set of targeting oligonucleotide probes comprises probes targeting at least two of MAP2K2, PPAP2B, CNBP, FIG. 4, TTC19, PAPL, RN7SL300P, or ITGAV. In some embodiments, the set of targeting oligonucleotide probes comprises probes targeting at least three of MAP2K2, PPAP2B, CNBP, FIG. 4, TTC19, PAPL, RN7SL300P, or ITGAV. In some embodiments, the set of targeting oligonucleotide probes comprises probes targeting at least 4, 5, 6, 7, or 8 of MAP2K2, PPAP2B, CNBP, FIG. 4, TTC19, PAPL, RN7SL300P, or ITGAV. In some embodiments, the set of targeting oligonucleotide probes comprises probes targeting MAP2K2, PPAP2B, CNBP, FIG. 4, TTC19, PAPL, RN7SL300P, ITGAV, or combinations thereof. In some embodiments, the set of targeting oligonucleotide probes comprises probes targeting MAP2K2, PPAP2B, CNBP, FIG. 4, TTC19, PAPL, RN7SL300P, or ITGAV.

In some embodiments, the set of targeting oligonucleotide probes comprises probes targeting at least one of GTF3C6, LINC01703, C1orf131, or XRCC2. In some embodiments, the set of targeting oligonucleotide probes comprises probes targeting at least two of GTF3C6, LINC01703, C1orf131, or XRCC2. In some embodiments, the set of targeting oligonucleotide probes comprises probes targeting at least three of GTF3C6, LINC01703, C1orf131, or XRCC2. In some embodiments, the set of targeting oligonucleotide probes comprises probes targeting at least one of GTF3C6, LINC01703, C1orf131, XRCC2, or combinations thereof. In some embodiments, the set of targeting oligonucleotide probes comprises probes targeting GTF3C6, LINC01703, C1orf131, or XRCC2.

In some embodiments, the set of targeting oligonucleotide probes comprises probes targeting at least one of ARHGEF10, CLDN23, C12orf36, and SPATA4, at least one of MAP2K2, PPAP2B, CNBP, FIG. 4, TTC19, PAPL, or RN7SL300P, and at least one of GTF3C6, LINC01703, C1orf131, or XRCC2. In some embodiments, the set of targeting oligonucleotide probes comprises probes targeting at least two of ARHGEF10, CLDN23, C12orf36, and SPATA4, at least two of MAP2K2, PPAP2B, CNBP, FIG. 4, TTC19, PAPL, or RN7SL300P, and at least two of GTF3C6, LINC01703, C1orf131, or XRCC2.

In some embodiments, the set of targeting oligonucleotide probes comprises probes targeting at least one of ARHGEF10, CLDN23, C12orf36, SPATA4, MAP2K2, PPAP2B, CNBP, FIG. 4, TTC19, PAPL, RN7SL300P, GTF3C6, LINC01703, C1orf131, XRCC2, or combinations thereof. In some embodiments, the set of targeting oligonucleotide probes comprises probes targeting any one of ARHGEF10, CLDN23, C12orf36, SPATA4, MAP2K2, PPAP2B, CNBP, FIG. 4, TTC19, PAPL, RN7SL300P, GTF3C6, LINC01703, C1orf131, XRCC2. In some embodiments, the set of targeting oligonucleotide probes comprises probes targeting ARHGEF10, CLDN23, C12orf36, SPATA4, MAP2K2, PPAP2B, CNBP, FIG. 4, TTC19, PAPL, RN7SL300P, GTF3C6, LINC01703, C1orf131, XRCC2.

In some embodiments, the kit further comprises reagents and instructions for enriching the biological sample obtained from the subject for tumor cells.

In some embodiments, the reagents comprise antibody-conjugated magnetic beads, EpCAM-conjugated magnetic beads, or a combination thereof to isolate tumor cells from non-tumor cells in the biological sample. The enrichment can be achieved by contacting the biological sample with an agent (e.g., antibody-conjugated magnetic beads, EpCAM-conjugated magnetic beads) to isolate tumor cells from non-tumor cells in the biological sample to enrich the sample for tumor cells. In some embodiments, the agent is an EpCAM-conjugated magnetic beads. In some embodiments, the agent is an EpCAM-conjugated non-magnetic beads such as one used in flow-cytometry assays or immunohistochemistry assays.

In some embodiments, the kit further comprising reagents and instructions for: (i) attaching a detectable label to the tagged DNA fragments to produce labeled fragments; and (ii) contacting the detectable labeled fragments to the set of oligonucleotide probes.

In some embodiments, the kit further comprising instructions for determining a prognostic score indicative of the subject's responsiveness to one or more treatment modalities based on a differential score of the first epigenetic signature value and the second epigenetic value.

In some embodiments, the instruction comprises normalizing the differential value with at least one of a positive control value and a negative control value to obtain the prognostic score.

In some embodiments, the prognostic score is at least 0.6. In some embodiments, the prognostic score is less than 0.6.

In some embodiments, the kit further comprises reagents and instructions for detecting nuclear localization of a transcription factor selected from any one of transcription factors in Tables 2A and 2B.

In some embodiments, the transcription factors are selected from ZKSCAN1, EPAS1, RUNX2, ZNF410, MAFF, RREB1, NR3C2, SMAD1, RUNX1, ZNF32, ZSCAN4, HOXB1, POU3F1, ZBTB3, CLOCK, TCF15, GCM1, HINFP, CGBP, MYPOP, ZNF384, GMEB2, E2F5, AC012531.1, ZBTB7B, HOXC9, HNF4G, CREB1, ATF2, E2F2, SP3, ARID5A, ZFP161, OTP, PBX3, ZBTB33, ONECUT3, ONECUT3, DLX2, HNF4A, PRRX1, TCFL5, HOXB7, IRF6, GRHL1, FOXD2, ISL1, MLL, GATA2, GATA1, HMBOX1, NRF1, ZFHX3, ONECUT1, TET1, E2F3, DNMT1, CTCFL, CTCF, HNF1B, and HNF1A.

In some embodiments, the transcription factors are selected from at least 1, 2, 3, 4, 5, 6, 7, 8, 9, or 10 of transcription factors ZKSCAN1, EPAS1, RUNX2, ZNF410, MAFF, RREB1, NR3C2, SMAD1, RUNX1, ZNF32, ZSCAN4, HOXB1, POU3F1, ZBTB3, CLOCK, TCF15, GCM1, HINFP, CGBP, MYPOP, ZNF384, GMEB2, E2F5, AC012531.1, ZBTB7B, HOXC9, HNF4G, CREB1, ATF2, E2F2, SP3, ARID5A, ZFP161, OTP, PBX3, ZBTB33, ONECUT3, ONECUT3, DLX2, HNF4A, PRRX1, TCFL5, HOXB7, IRF6, GRHL1, FOXD2, ISL1, MLL, GATA2, GATA1, HMBOX1, NRF1, ZFHX3, ONECUT1, TET1, E2F3, DNMT1, CTCFL, CTCF, HNF1B, and HNF1A.

In some embodiments, the transcription factors are selected ZKSCAN1A, HNF1B, or a combination thereof.

In some embodiments, the kit further comprises reagents and instructions for predicting a long duration of disease-free survival when the first epigenetic signature value is significantly lower than the second epigenetic signature value and/or predicting a short duration of disease-free survival when the first epigenetic signature value is significantly higher than the second epigenetic value.

In some embodiments, the kit further comprises reagents and instructions for determining a first phenotype and second phenotype of the subject's responsiveness to one or more treatment modalities.

In some embodiments, the first phenotype is recurrence of a cancer within 6 months, or 1, 2, 3, 4, or 5 years of surgical resection and/or the second phenotype is non-recurrence of a cancer within 6 months, or 1, 2, 3, 4, or 5 years of surgical resection. In some embodiments, the first phenotype is recurrence of a cancer within one year of surgical resection and the second phenotype is non-recurrence of a cancer within one year of surgical resection.

In some embodiments, the first phenotype is non-responder to a cancer therapy. In some embodiments, the second phenotype is responder to a cancer therapy. In some embodiments, the cancer therapy is selected from chemotherapy, immunotherapy, radiation, or combinations thereof.

In some embodiments, the second phenotype is having a median disease-free survival (DFS) of at least of 1, 2, 3, 4, 5, 6, 7, 8, 9, 10, or more years.

In some embodiments, the second phenotype is having a median disease-free survival (DFS) of at least 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, or more days.

In some embodiments, the second phenotype is having a median disease-free survival (DFS) of between 50 to 1500 days, between 100 to 1000 days, between 300 to 800 days, between 200 to 500 days, or between 400 to 600 days. In some embodiments, the first phenotype is having a median disease-free survival of at least 350 days.

In some embodiments, the first phenotype is having a median disease-free survival of less than 5, 4, 2, 3, 1 or less years. In some embodiments, the first phenotype is having a median disease-free survival of less than 12, 11, 10, 9, 8, 7, 6, 5, 4, 3, 2, 1 or less months. In some embodiments, the first phenotype is having a median disease-free survival of less than 350, 300, 250, 200, 150, 100, 50, 10, or less days. In some embodiments, the first phenotype is having a median disease-free survival of less than 50 days.

In some embodiments, the first phenotype is having a median disease-free survival of between 1 to 350 days, between 50-300 days, between 10-100 days, or between 40 to 250 days.

In some embodiments, the biological sample used to generate the ATAC-library, which can be used as ATAC-qPCR array template DNA, comprises treatment-naïve malignant cells obtained from a subject. In some embodiments, the subject is a treatment naïve patient who has not received the one or more treatment modalities.

In some embodiments, the subject is under treatment or has been treated with the one or more treatment modalities.

In some embodiments, the one or more treatment modalities are selected from resecting cancerous tissue, neo-adjuvant chemotherapy, adjuvant chemotherapy, immunotherapy, an epigenetic drug, or combinations thereof. In some embodiments, the epigenetic drug is selected from DNMT inhibitor, an HDAC inhibitor, an EZH2 inhibitor, or combinations thereof. In some embodiments, the DNMT inhibitor is azacytidine or decitabine. In some embodiments, the HDAC inhibitor is vorinostat or romidepsin.

In some embodiments, the neo-adjuvant chemotherapy and/or adjuvant chemotherapy is selected from gemcitabine, nab-paclitaxel, fluorouracil (5-FU), irinotecan, oxaliplatin, leucovorin, capecitabine, cisplatin, or combinations thereof.

In some embodiments, the biological sample is selected from a tumor biopsy or surgically resected tumor specimen.

In some embodiments, obtaining the targeted accessible chromatin region fragments does not include sequencing the tagged fragments or amplicons thereof. In some embodiments, the amplified targeted accessible chromatin fragments comprise a mean size of about 50 bp to about 1100 bp, about 100 bp to about 350 bp, about 150 bp to about 800 bp, about 80 bp to about 150 bp, or about 150 bp to about 400 bp. In some embodiments, the amplified targeted accessible chromatin fragments comprise a mean size of 80 bp, about 100 bp, about 120 bp, about 150 bp, about 180 bp, about 200 bp, about 250 bp, about 300 bp, about 400 bp, about 500 bp, about 600 bp, about 700 bp, about 800 bp, about 900 bp, about 1000 bp, about 1100 bp, or longer. In some embodiments, the amplified targeted accessible chromatin fragments comprise a mean size of about 120 bp.

### 8. EXAMPLES

### 8.1 Example 1: PDAC Recurrence

A prospective cohort of treatment-naïve, surgically resected tumors from 54 PDAC patients was collected (n=54). PDAC malignant cells from freshly resected tumors were sorted using EpCAM-conjugated magnetic beads. Both EpCAM+ and EpCAM-cells from each of the tumors were collected. The canonical variant allele frequencies (VAF) of pancreatic cancer driver genes KRAS and TP53 in the EpCAM+ cells were both dramatically higher than that of the EpCAM-cells (P<0.001, t-test) confirming the effective enrichment of malignant epithelial cells in EpCAM+subpopulation of the same tumor. This enrichment was further confirmed by transcriptome analysis, which demonstrated overexpression of epithelial genes in the EpCAM+subpopulation, with corresponding expression of immune cell and collagen genes in the EpCAM-subpopulation.

Assay for Transposase-Accessible Chromatin sequencing (ATAC-seq) was performed on the EpCAM+ cells to interrogate genome-wide chromatin accessibility and associated differentially accessible TF binding sites. A global atlas of 121,697 peaks with median width of 505 bp, where each peak was reproducible in replicate ATAC-seq libraries for at least one patient was assembled. Saturation analysis was performed to estimate incremental new peak discovery associated with step-wise increases in sample size and confirmed that a sample size of n=40 approached saturating coverage.

Follow-up clinical data were available for 36 out of 40 patients included in the atlas. Nineteen (19) of 36 patients were at least 365 days post-treatment, among whom 9 patients (47.4%) had recurred (DFS≤1 year, referred to as the recurrent group), and 10 patients had no recurrence (DFS>1 year; maximum of 660 days, referred to as the non-recurrent group). The latter group, however, was expected to be mixture of long-term survivors and others who will recur in 2-5 years. For the discovery analyses, 3 patients who did not receive any adjuvant chemotherapy were excluded, leaving 16 patients (6 recurrent and 10 non-recurrent). A multi-factor generalized linear model was then used to identify significant differential chromatin accessibility events between the recurrent versus non-recurrent groups, while controlling for the effects of read depth and margin status.

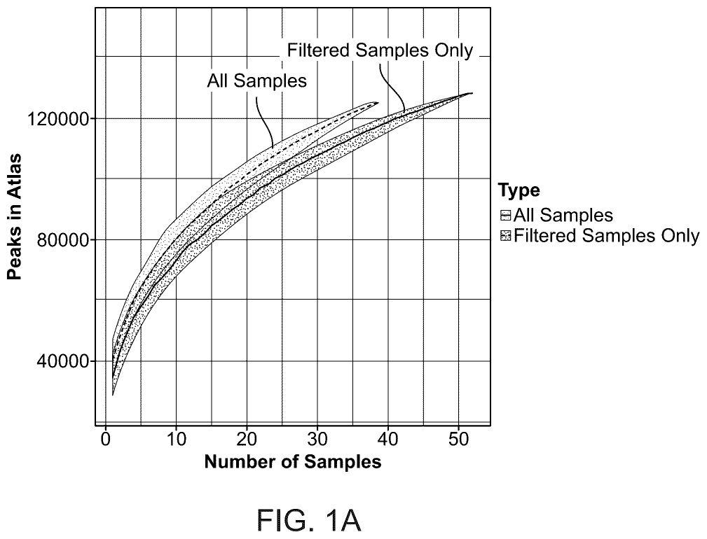

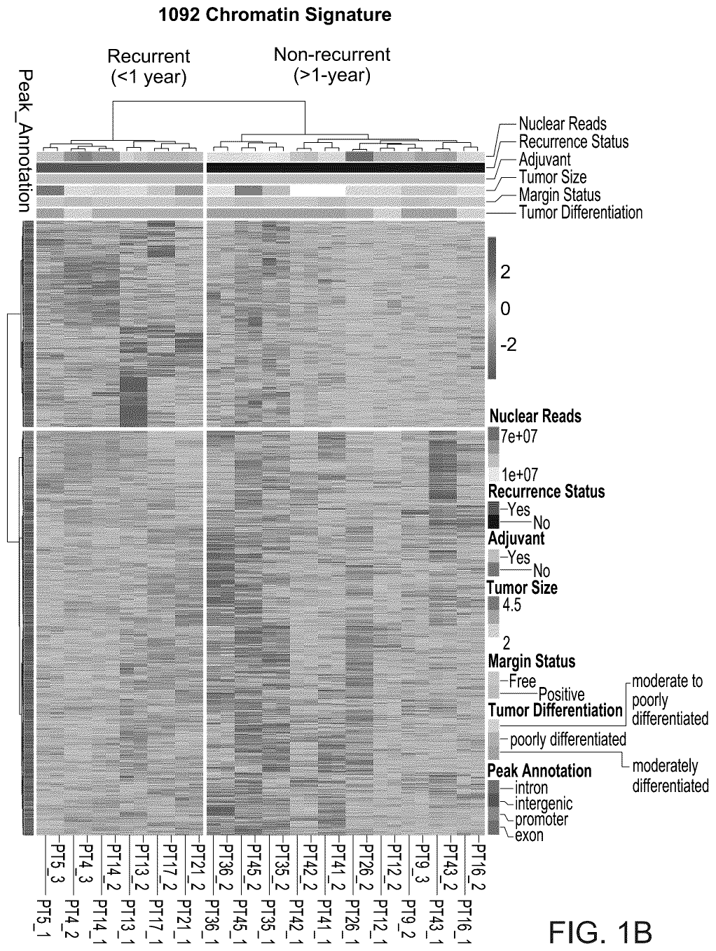

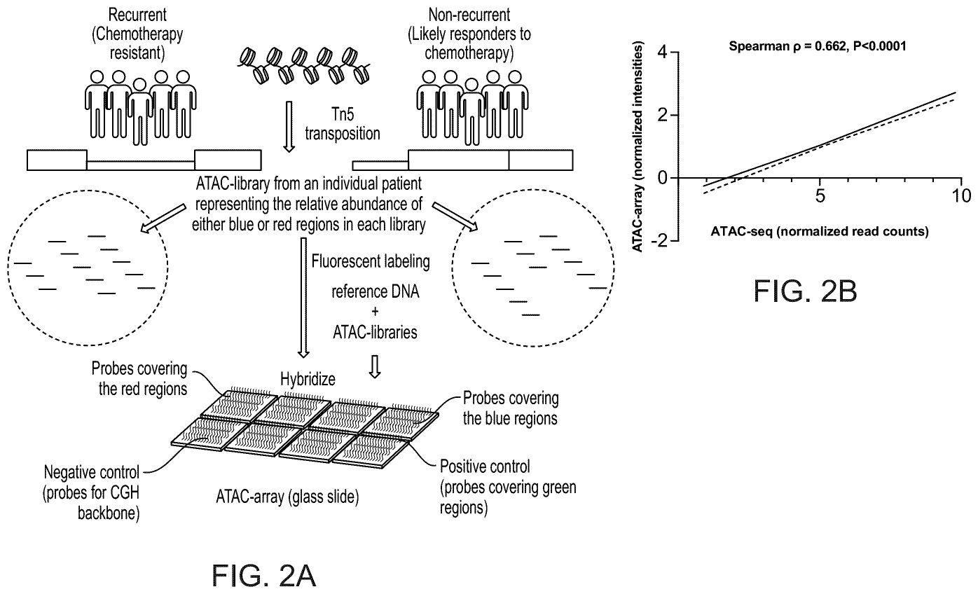

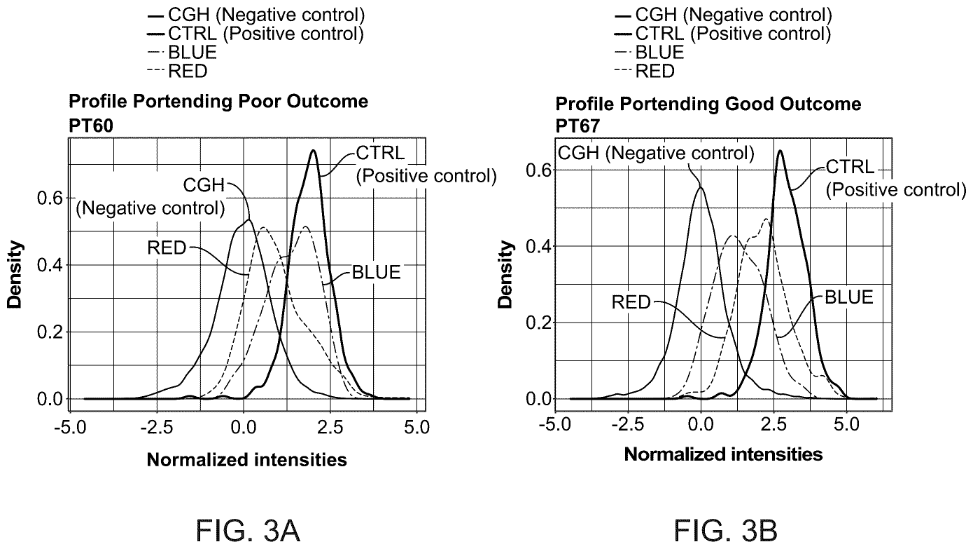

More than one thousand (1092) open chromatin peaks were identified as being differentially accessible (absolute log₂ fold change >1 and FDR-adjusted P<0.001) between the patients who recurred within a year of surgery and the patients who did not recur (maximum follow-up of 660 days) by ATAC-seq as in FIGS. 1A-1B. The differentially accessible chromatin regions are listed in Table 1.

TABLE 1

| | | | | | | | | Peak |
|---|---|---|---|---|---|---|---|---|
| | | | | | Gene | Fold | P-value | width |
| Chr | Start | End | Annotation | Transcript_id | symbol | Change | (adj) | (bp) |
| 17 | 52977819 | 52978913 | promoter | ENST00000575909 | TOM1L1 | −1.01 | 2.2E−06 | 1094 |
| 10 | 134254015 | 134254324 | promoter | ENST00000450206 | RP11-432J24.3 | 1.56 | 9.0E−04 | 309 |
| 10 | 1506248 | 1507137 | intron | ENST00000381312 | ADARB2 | 1.30 | 5.1E−05 | 889 |
| 9 | 27625883 | 27626853 | intergenic | ENST00000400348 | CTAGE12P | −1.38 | 2.1E−08 | 970 |
| 14 | 105698636 | 105698888 | intron | ENST00000550208 | BRF1 | 1.35 | 1.6E−05 | 252 |
| 7 | 401254 | 401879 | intergenic | ENST00000515213 | AC226118.1 | 1.31 | 5.8E−04 | 625 |
| 7 | 4652611 | 4652868 | intergenic | ENST00000446823 | FOXK1 | 1.05 | 8.9E−04 | 257 |
| 22 | 23744503 | 23745176 | promoter | ENST00000420968 | ZDHHC8P1 | −1.04 | 7.1E−04 | 673 |
| X | 3615234 | 3616144 | intron | ENST00000262848 | PRKX | −1.12 | 7.8E−04 | 910 |
| 17 | 104433 | 104716 | intron | ENST00000570638 | RPH3AL | 1.45 | 9.4E−04 | 283 |
| 2 | 1420825 | 1421839 | intron | ENST00000382198 | TPO | 1.08 | 8.6E−05 | 1014 |
| 16 | 12895395 | 12895819 | promoter | ENST00000539677 | CPPED1 | 1.20 | 1.6E−04 | 424 |
| 16 | 89495481 | 89495932 | intron | ENST00000566973 | ANKRD11 | 1.11 | 6.9E−04 | 451 |
| 16 | 1389753 | 1390364 | intron | ENST00000421665 | BAIAP3 | 1.24 | 3.3E−04 | 611 |
| 6 | 2504483 | 2504975 | intergenic | ENST00000606884 | GMDS-AS1 | 1.18 | 2.4E−04 | 492 |
| 2 | 2016399 | 2016710 | intron | ENST00000479156 | MYT1L | 1.82 | 3.4E−08 | 311 |
| 17 | 75480980 | 75481368 | intron | ENST00000585638 | 9-Sep | 1.29 | 2.8E−04 | 388 |
| 22 | 30601850 | 30602161 | promoter | ENST00000432360 | RP3-438O4.4 | 1.44 | 6.3E−04 | 311 |
| 1 | 3594879 | 3595126 | intron | ENST00000357733 | TP73 | 1.36 | 1.2E−04 | 247 |
| 11 | 460271 | 461039 | promoter | ENST00000526878 | PTDSS2 | 1.32 | 1.2E−04 | 768 |
| 9 | 138020994 | 138021355 | intergenic | ENST00000371796 | OLFM1 | 1.35 | 2.4E−04 | 361 |
| 12 | 123933887 | 123934119 | intergenic | ENST00000605712 | RP11-972P1.8 | 1.34 | 2.2E−04 | 232 |
| X | 10087464 | 10087962 | intron | ENST00000454666 | WWC3 | −1.50 | 7.4E−05 | 498 |
| 18 | 56179682 | 56180292 | intron | ENST00000361673 | ALPK2 | 1.07 | 2.5E−04 | 610 |
| 18 | 18970975 | 18971689 | intron | ENST00000584611 | RP11-296E23.1 | −1.05 | 2.6E−04 | 714 |
| 17 | 106478 | 107040 | intron | ENST00000570638 | RPH3AL | 1.30 | 1.9E−04 | 562 |
| 18 | 20512914 | 20514073 | promoter | ENST00000578831 | RP11-739L10.1 | −1.03 | 3.8E−04 | 1159 |
| 11 | 70454 | 70919 | intergenic | ENST00000519787 | RP11-304M2.1 | 1.54 | 5.0E−04 | 465 |
| 2 | 242869770 | 242871341 | intron | ENST00000429947 | AC131097.3 | 1.15 | 3.2E−04 | 1571 |
| 19 | 40421670 | 40422156 | intron | ENST00000221347 | FCGBP | 1.56 | 4.3E−04 | 486 |
| 11 | 92438452 | 92438798 | intron | ENST00000525166 | FAT3 | 1.03 | 5.5E−04 | 346 |
| 1 | 59246541 | 59247091 | exon | ENST00000371222_43680 | | −1.40 | 4.0E−04 | 550 |
| 18 | 6413976 | 6415319 | promoter | ENST00000580162 | L3MBTL4 | −1.20 | 3.9E−04 | 1343 |
| 14 | 67878683 | 67879198 | promoter | ENST00000557388 | PLEK2 | −1.06 | 2.8E−05 | 515 |
| 10 | 51566723 | 51567262 | promoter | ENST00000414907 | NCOA4 | −1.18 | 2.3E−04 | 539 |
| 11 | 128149728 | 128150176 | intergenic | ENST00000608492 | RP11-702B10.1 | 1.57 | 1.7E−04 | 448 |
| 20 | 5344571 | 5345402 | intergenic | ENST00000363443 | RNA5-8SP7 | 1.23 | 1.0E−04 | 831 |
| 10 | 106087848 | 106088405 | promoter | ENST00000358187 | ITPRIP | 1.27 | 1.3E−05 | 557 |
| 20 | 17540069 | 17540372 | promoter | ENST00000377868 | BFSP1 | 1.24 | 2.4E−04 | 303 |
| 7 | 66017307 | 66017790 | intron | ENST00000445080 | GS1-124K5.12 | 1.08 | 1.2E−04 | 483 |
| 2 | 9445224 | 9446284 | intron | ENST00000315273 | ASAP2 | 1.04 | 9.6E−04 | 1060 |
| 4 | 84255872 | 84256464 | promoter | ENST00000513463 | HPSE | −1.06 | 8.7E−05 | 592 |
| 6 | 78359808 | 78360616 | intergenic | ENST00000602452 | MEI4 | −1.49 | 2.7E−04 | 808 |
| 18 | 21017554 | 21018179 | promoter | ENST00000399707 | TMEM241 | −1.15 | 3.3E−04 | 625 |
| 19 | 4084177 | 4084702 | intergenic | ENST00000262948 | MAP2K2 | 1.05 | 2.6E−04 | 525 |
| 7 | 36555230 | 36555979 | promoter | ENST00000471806 | AOAH | 1.10 | 1.5E−04 | 749 |
| 3 | 18799504 | 18799922 | intron | ENST00000425799 | AC144521.1 | −1.11 | 1.4E−10 | 418 |
| 8 | 86375420 | 86376638 | promoter | ENST00000517697 | RP11-317J10.2 | −1.13 | 3.7E−09 | 1218 |
| 16 | 33345278 | 33346583 | promoter | ENST00000568752 | RP11-989E6.10 | 1.14 | 2.9E−04 | 1305 |
| 7 | 63220603 | 63221633 | intergenic | ENST00000605464 | CICP24 | 1.21 | 1.2E−04 | 1030 |
| 2 | 87651678 | 87651939 | intergenic | ENST00000444323 | AC068279.3 | 1.16 | 2.8E−04 | 261 |
| 22 | 19434714 | 19435523 | promoter | ENST00000333059 | C22orf39 | −1.03 | 9.5E−04 | 809 |
| 3 | 126945866 | 126946636 | intergenic | ENST00000492080 | RP11-305F5.2 | 1.05 | 4.0E−04 | 770 |
| 11 | 2011127 | 2011556 | promoter | ENST00000419080 | MRPL23-AS1 | 1.54 | 1.0E−05 | 429 |
| 11 | 119612823 | 119613563 | intergenic | ENST00000533253 | CTD-2523D13.2 | 1.14 | 2.5E−04 | 740 |
| 1 | 1293633 | 1294442 | promoter | ENST00000445648 | MXRA8 | 1.33 | 1.9E−08 | 809 |
| 11 | 102364324 | 102364756 | intron | ENST00000529278 | RP11-315O6.2 | −1.07 | 2.8E−05 | 432 |
| 1 | 117900726 | 117901380 | intergenic | ENST00000604156 | RP11-188D8.1 | 1.38 | 1.1E−04 | 654 |
| 7 | 102091876 | 102092500 | exon | ENST00000356387_249477 | | 1.38 | 3.5E−04 | 624 |
| 10 | 92690759 | 92691502 | intergenic | ENST00000364734 | RNU6-740P | 1.09 | 6.7E−04 | 743 |
| 10 | 135342280 | 135342918 | promoter | ENST00000599428 | AL161645.2 | 1.69 | 7.1E−06 | 638 |
| 12 | 124857925 | 124858331 | intron | ENST00000448614 | NCOR2 | 1.14 | 4.6E−04 | 406 |
| 18 | 9474785 | 9475995 | promoter | ENST00000383432 | RALBP1 | −1.03 | 3.7E−04 | 1210 |
| X | 20396229 | 20397054 | intergenic | ENST00000517169 | RN7SKP183 | −1.25 | 4.1E−04 | 825 |
| 1 | 2111576 | 2112325 | intron | ENST00000505322 | PRKCZ | 1.32 | 4.8E−04 | 749 |
| 4 | 71450043 | 71450658 | intergenic | ENST00000322937 | AMBN | −1.15 | 9.9E−05 | 615 |
| 11 | 1086528 | 1086937 | intron | ENST00000359061 | MUC2 | 1.19 | 7.0E−05 | 409 |
| 2 | 10539305 | 10539660 | intron | ENST00000419810 | HPCAL1 | 1.24 | 6.7E−05 | 355 |
| 16 | 33348985 | 33350971 | promoter | ENST00000568752 | RP11-989E6.10 | 1.18 | 4.8E−05 | 1986 |
| 21 | 47410321 | 47410540 | intron | ENST00000361866 | COL6A1 | 1.19 | 3.5E−04 | 219 |
| 19 | 36774890 | 36775141 | intergenic | ENST00000586345 | CTD-3162L10.1 | 1.18 | 9.4E−04 | 251 |
| 19 | 36790418 | 36790944 | intergenic | ENST00000586345 | CTD-3162L10.1 | 1.40 | 1.8E−05 | 526 |
| 19 | 36791007 | 36791167 | intergenic | ENST00000586345 | CTD-3162L10.1 | 1.61 | 2.0E−04 | 160 |
| 16 | 32297453 | 32298796 | intergenic | ENST00000568567 | RP11-17M15.2 | 1.16 | 3.0E−05 | 1343 |
| 19 | 36785355 | 36785870 | intergenic | ENST00000586345 | CTD-3162L10.1 | 1.28 | 7.8E−04 | 515 |
| 12 | 9558298 | 9559027 | intron | ENST00000540982 | RP11-599J14.2 | 1.17 | 4.1E−04 | 729 |
| 3 | 125726687 | 125727535 | promoter | ENST00000504118 | SLC41A3 | 1.08 | 6.4E−04 | 848 |
| 10 | 129058797 | 129060504 | intron | ENST00000464466 | DOCK1 | 1.09 | 3.6E−04 | 1707 |
| 2 | 87642804 | 87643025 | intergenic | ENST00000444323 | AC068279.3 | 1.10 | 3.8E−04 | 221 |
| 1 | 11296642 | 11297523 | intron | ENST00000361445 | MTOR | 1.43 | 9.5E−04 | 881 |
| 12 | 31871662 | 31871945 | intron | ENST00000509386 | AMN1 | 1.78 | 8.9E−05 | 283 |
| 5 | 1521927 | 1522688 | promoter | ENST00000514484 | LPCAT1 | 1.23 | 2.7E−06 | 761 |
| 1 | 4692485 | 4692949 | intergenic | ENST00000378190 | AJAP1 | 1.15 | 9.3E−04 | 464 |
| 1 | 66655817 | 66656717 | intron | ENST00000412480 | PDE4B | 1.25 | 5.2E−04 | 900 |
| 1 | 16970486 | 16970660 | promoter | ENST00000362058 | CROCCP2 | 1.01 | 7.5E−05 | 174 |
| 19 | 6677696 | 6678735 | promoter | ENST00000601475 | C3 | 1.13 | 5.3E−04 | 1039 |
| 1 | 56933904 | 56934476 | intergenic | ENST00000371250 | PPAP2B | 1.32 | 2.7E−04 | 572 |
| 1 | 4693158 | 4693721 | intergenic | ENST00000378190 | AJAP1 | 1.10 | 2.8E−04 | 563 |
| 12 | 123333197 | 123333659 | promoter | ENST00000536772 | HIP1R | 1.13 | 9.0E−04 | 462 |
| 1 | 193406539 | 193407729 | intergenic | ENST00000420807 | LINC01031 | 1.07 | 5.6E−04 | 1190 |
| 3 | 121723306 | 121724477 | promoter | ENST00000462014 | ILDR1 | 1.05 | 9.6E−04 | 1171 |
| 2 | 209676292 | 209676942 | intron | ENST00000419079 | PTH2R | 1.38 | 3.6E−04 | 650 |
| 12 | 3306839 | 3307985 | intron | ENST00000011898 | TSPAN9 | 1.42 | 5.2E−04 | 1146 |
| 11 | 94615832 | 94616486 | intron | ENST00000545958 | RP11-856F16.2 | 1.10 | 6.5E−04 | 654 |
| 1 | 3604957 | 3605264 | promoter | ENST00000378280 | TP73 | 1.32 | 3.3E−04 | 307 |
| 2 | 1391878 | 1392649 | intron | ENST00000497517 | TPO | 1.05 | 5.7E−04 | 771 |
| 1 | 811189 | 812119 | promoter | ENST00000427857 | FAM41C | 1.07 | 4.0E−04 | 930 |
| 19 | 38468627 | 38469128 | intron | ENST00000476317 | SIPA1L3 | 1.28 | 8.3E−04 | 501 |
| 1 | 238292984 | 238293512 | intergenic | ENST00000445891 | YWHAQP9 | 1.34 | 6.1E−05 | 528 |
| 13 | 39210941 | 39211343 | intergenic | ENST00000447765 | PRDX3P3 | −1.33 | 4.0E−04 | 402 |
| 5 | 42811882 | 42812507 | promoter | ENST00000508937 | SEPP1 | −1.05 | 1.9E−05 | 625 |
| 2 | 1560598 | 1560978 | intron | ENST00000438247 | AC144450.1 | 1.15 | 1.5E−04 | 380 |
| 12 | 132815639 | 132815889 | intron | ENST00000328957 | GALNT9 | 1.20 | 9.9E−04 | 250 |
| 9 | 115846414 | 115847424 | intergenic | ENST00000439875 | FAM225B | 1.27 | 1.8E−04 | 1010 |
| 19 | 54613041 | 54613311 | promoter | ENST00000482960 | NDUFA3 | 1.72 | 2.1E−06 | 270 |
| 2 | 239204444 | 239205433 | intergenic | ENST00000437372 | AC012485.2 | 1.28 | 1.9E−04 | 989 |
| 11 | 397903 | 398909 | promoter | ENST00000526971 | PKP3 | 1.04 | 2.5E−04 | 1006 |
| 1 | 1912821 | 1913709 | intron | ENST00000468610 | C1orf222 | 1.43 | 3.6E−06 | 888 |
| 19 | 37782096 | 37782418 | intergenic | ENST00000586442 | CTD-3220F14.1 | 1.63 | 6.4E−04 | 322 |
| 2 | 11776847 | 11777488 | intron | ENST00000396123 | GREB1 | 1.34 | 6.0E−05 | 641 |
| 12 | 132813440 | 132813985 | intron | ENST00000328957 | GALNT9 | 1.07 | 6.4E−04 | 545 |
| 1 | 110663023 | 110663256 | intergenic | ENST00000334179 | UBL4B | 1.20 | 8.9E−04 | 233 |
| 7 | 155893958 | 155894405 | intergenic | ENST00000384333 | Y_RNA | 1.04 | 4.3E−04 | 447 |
| 20 | 36202030 | 36202951 | intergenic | ENST00000423261 | GLRXP | 1.04 | 5.1E−04 | 921 |
| 1 | 228778123 | 228778480 | promoter | ENST00000365055 | RNA5S15 | 1.08 | 1.6E−05 | 357 |
| 1 | 4503130 | 4503709 | intergenic | ENST00000423197 | RP5-1166F10.1 | 1.15 | 8.4E−04 | 579 |
| 20 | 62781169 | 62781776 | promoter | ENST00000360149 | MYT1 | 1.23 | 6.1E−04 | 607 |
| 18 | 8890911 | 8891510 | intergenic | ENST00000359865 | SOGA2 | −1.21 | 4.2E−04 | 599 |
| 14 | 64330729 | 64331355 | promoter | ENST00000556725 | SYNE2 | −1.09 | 1.1E−06 | 626 |
| 3 | 195509793 | 195510202 | exon | ENST00000478156_152007 | | 1.45 | 8.0E−05 | 409 |
| 11 | 134831292 | 134832253 | intergenic | ENST00000528497 | RP11-555G19.1 | 1.30 | 3.1E−04 | 961 |
| 5 | 11443246 | 11443549 | intron | ENST00000508761 | CTNND2 | 1.41 | 3.7E−04 | 303 |
| 9 | 140244387 | 140245281 | intron | ENST00000484392 | EXD3 | 1.09 | 6.4E−04 | 894 |
| 19 | 39648485 | 39649257 | promoter | ENST00000599657 | PAK4 | 1.37 | 6.4E−04 | 772 |
| 10 | 132271970 | 132272400 | intron | ENST00000439421 | RP11-540N6.1 | 1.08 | 5.1E−04 | 430 |
| 11 | 41553950 | 41554439 | intron | ENST00000526978 | RP11-124G5.3 | 1.17 | 9.1E−04 | 489 |
| 1 | 247292118 | 247293363 | intron | ENST00000476312 | ZNF124 | 1.35 | 3.0E−06 | 1245 |
| 20 | 61588799 | 61589615 | intron | ENST00000411611 | SLC17A9 | 1.10 | 7.6E−04 | 816 |
| 19 | 30056819 | 30058660 | intergenic | ENST00000335523 | VSTM2B | 1.09 | 5.4E−04 | 1841 |
| 19 | 49337650 | 49338689 | promoter | ENST00000595764 | HSD17B14 | 1.14 | 4.5E−04 | 1039 |
| 1 | 37259254 | 37259535 | intergenic | ENST00000373091 | GRIK3 | 1.12 | 9.6E−04 | 281 |
| 12 | 131780776 | 131781831 | promoter | ENST00000508505 | RP11-495K9.3 | 1.00 | 9.5E−04 | 1055 |
| 19 | 32223716 | 32224929 | intergenic | ENST00000365024 | RNU6-967P | 1.19 | 4.6E−04 | 1213 |
| 2 | 211054239 | 211055494 | intron | ENST00000412065 | AC006994.2 | −1.02 | 8.6E−04 | 1255 |
| 1 | 37449602 | 37450029 | intron | ENST00000373093 | GRIK3 | 1.00 | 9.5E−04 | 427 |
| 1 | 40128961 | 40129465 | intron | ENST00000235628 | NT5C1A | 1.12 | 2.4E−04 | 504 |
| 14 | 38677991 | 38678610 | promoter | ENST00000267377 | SSTR1 | −1.26 | 6.9E−04 | 619 |
| 1 | 4769995 | 4770757 | promoter | ENST00000466761 | AJAP1 | 1.13 | 4.4E−04 | 762 |
| 21 | 47318748 | 47319014 | promoter | ENST00000468429 | PCBP3 | 1.07 | 5.3E−04 | 266 |
| 19 | 34280154 | 34280686 | intergenic | ENST00000587658 | KCTD15 | 1.16 | 3.4E−04 | 532 |
| X | 23522303 | 23522698 | intergenic | ENST00000458766 | snoU13 | −1.03 | 9.0E−04 | 395 |
| 13 | 108686039 | 108687002 | intergenic | ENST00000375915 | FAM155A | −1.67 | 1.9E−10 | 963 |
| 21 | 16512741 | 16513203 | intergenic | ENST00000449746 | AF127577.12 | −1.46 | 1.2E−05 | 462 |
| 4 | 69817171 | 69817631 | promoter | ENST00000251566 | UGT2A3 | −1.81 | 3.2E−07 | 460 |
| 1 | 224136990 | 224138052 | promoter | ENST00000424045 | CICP5 | 1.03 | 3.7E−04 | 1062 |
| 11 | 1796937 | 1797410 | intergenic | ENST00000449749 | AC068580.7 | 1.36 | 3.0E−04 | 473 |
| 11 | 132947949 | 132948284 | intron | ENST00000529038 | OPCML | 1.09 | 6.7E−04 | 335 |
| 1 | 1276059 | 1277202 | intron | ENST00000472445 | DVL1 | 1.09 | 6.2E−04 | 1143 |
| X | 2526973 | 2527761 | promoter | ENST00000527459 | CD99P1 | −1.15 | 1.8E−04 | 788 |
| 14 | 72448205 | 72449425 | intron | ENST00000402788 | RGS6 | 1.05 | 1.9E−04 | 1220 |
| 18 | 21718709 | 21719338 | promoter | ENST00000327201 | CABYR | −1.41 | 1.3E−05 | 629 |
| 18 | 21851298 | 21852369 | promoter | ENST00000585247 | OSBPL1A | −1.02 | 9.0E−05 | 1071 |
| 4 | 170121436 | 170122132 | promoter | ENST00000510225 | RP11-327O17.2 | −1.07 | 2.8E−04 | 696 |
| 14 | 77589823 | 77590311 | intron | ENST00000557752 | RP11-463C8.4 | −1.04 | 2.7E−04 | 488 |
| 3 | 65938946 | 65939447 | promoter | ENST00000460754 | MAGI1-IT1 | −1.18 | 1.7E−04 | 501 |
| 12 | 10826411 | 10827032 | promoter | ENST00000541561 | STYK1 | −1.10 | 3.7E−04 | 621 |
| 13 | 76334271 | 76334966 | promoter | ENST00000465261 | LMO7 | −1.05 | 1.4E−04 | 695 |
| 4 | 106816201 | 106816854 | promoter | ENST00000503451 | NPNT | −1.33 | 3.6E−07 | 653 |
| 8 | 119890394 | 119891274 | intergenic | ENST00000297350 | TNFRSF11B | −1.32 | 3.8E−05 | 880 |
| 18 | 3230353 | 3230820 | intron | ENST00000580139 | RP13-270P17.2 | −1.94 | 8.6E−05 | 467 |
| 4 | 23789895 | 23790557 | intron | ENST00000509702 | PPARGC1A | −1.28 | 2.2E−05 | 662 |
| 4 | 72978120 | 72978772 | intron | ENST00000358749 | NPFFR2 | −1.41 | 3.0E−10 | 652 |
| 6 | 53719637 | 53720021 | intron | ENST00000370882 | LRRC1 | −1.16 | 1.3E−04 | 384 |
| 18 | 59561256 | 59561922 | promoter | ENST00000588396 | RNF152 | −1.14 | 5.4E−05 | 666 |
| 5 | 158122586 | 158122909 | intergenic | ENST00000519890 | EBF1 | −1.36 | 6.3E−04 | 323 |
| 6 | 43894454 | 43895332 | intergenic | ENST00000422059 | RP5-1120P11.1 | −1.20 | 7.8E−06 | 878 |
| 17 | 38439964 | 38440892 | intron | ENST00000323571 | WIPF2 | −1.26 | 1.2E−04 | 928 |
| 4 | 103541806 | 103542546 | intergenic | ENST00000226574 | NFKB1 | −1.01 | 1.9E−07 | 740 |
| 5 | 170176920 | 170177427 | intron | ENST00000521965 | MIR4454 | −1.15 | 1.7E−04 | 507 |
| 4 | 25864064 | 25865011 | promoter | ENST00000513364 | SEL1L3 | −1.12 | 1.5E−04 | 947 |
| 12 | 15815552 | 15815954 | promoter | ENST00000540613 | EPS8 | −1.00 | 7.9E−05 | 402 |
| 19 | 11545786 | 11546623 | promoter | ENST00000586836 | CCDC151 | −1.05 | 9.7E−05 | 837 |
| X | 13006739 | 13007333 | intergenic | ENST00000451311 | TMSB4X | −1.36 | 1.0E−05 | 594 |
| 18 | 21692827 | 21693592 | promoter | ENST00000540918 | TTC39C | −1.64 | 1.9E−06 | 765 |
| 9 | 27371349 | 27371791 | intron | ENST00000603061 | MOB3B | −1.44 | 3.6E−09 | 442 |
| 18 | 29738048 | 29738594 | intron | ENST00000583696 | GAREM | −1.59 | 4.8E−04 | 546 |
| 3 | 18699491 | 18700274 | intron | ENST00000595388 | AC144521.1 | −1.65 | 5.8E−06 | 783 |
| 7 | 158995654 | 158996480 | intergenic | ENST00000437005 | PIP5K1P2 | 1.08 | 5.5E−04 | 826 |
| 12 | 15865506 | 15866240 | promoter | ENST00000543612 | EPS8 | −1.41 | 7.0E−07 | 734 |
| 4 | 155547868 | 155548555 | promoter | ENST00000499392 | LRAT | −1.02 | 6.5E−04 | 687 |
| 5 | 106810443 | 106811097 | intron | ENST00000505499 | EFNA5 | −1.08 | 3.5E−04 | 654 |
| 17 | 39956851 | 39957456 | intergenic | ENST00000355468 | LEPREL4 | −1.11 | 8.9E−04 | 605 |
| 18 | 68048808 | 68049145 | exon | ENST00000582251_572674 | | −1.16 | 1.3E−05 | 337 |
| 20 | 22471368 | 22471859 | intergenic | ENST00000420070 | LINC00261 | −1.14 | 8.0E−04 | 491 |
| 13 | 61989175 | 61989676 | promoter | ENST00000409204 | PCDH20 | −1.39 | 1.7E−05 | 501 |
| 5 | 78791005 | 78791692 | intron | ENST00000535690 | HOMER1 | −1.57 | 1.1E−08 | 687 |
| 1 | 157210261 | 157210939 | intergenic | ENST00000449345 | RP11-85G21.1 | −1.09 | 5.7E−04 | 678 |
| 17 | 56591826 | 56592157 | promoter | ENST00000582390 | MTMR4 | −1.12 | 3.1E−04 | 331 |
| 1 | 27240311 | 27240999 | promoter | ENST00000254227 | NR0B2 | −1.34 | 2.6E−04 | 688 |
| 4 | 149366324 | 149366956 | promoter | ENST00000344721 | NR3C2 | −1.25 | 2.5E−05 | 632 |
| 13 | 74861868 | 74862243 | promoter | ENST00000383890 | RNY1P5 | −1.51 | 9.2E−07 | 375 |
| 15 | 53745621 | 53746295 | intergenic | ENST00000567224 | WDR72 | −1.62 | 2.4E−08 | 674 |
| 7 | 87198356 | 87198910 | intron | ENST00000543898 | ABCB1 | −1.22 | 3.7E−05 | 554 |
| 4 | 38134715 | 38135185 | promoter | ENST00000492180 | TBC1D1 | −1.18 | 4.6E−05 | 470 |
| 18 | 77005558 | 77006476 | intron | ENST00000587878 | ATP9B | 1.10 | 2.1E−04 | 918 |
| 4 | 42658842 | 42659808 | promoter | ENST00000562054 | RP11-109E24.2 | −1.11 | 1.3E−05 | 966 |
| 8 | 128309764 | 128310584 | intron | ENST00000523825 | CASC8 | −1.23 | 2.4E−04 | 820 |
| 18 | 25236246 | 25236678 | intergenic | ENST00000584546 | RP11-739N10.1 | −1.75 | 1.7E−04 | 432 |
| 4 | 83316004 | 83316436 | intergenic | ENST00000503202 | IGBP1P4 | −1.15 | 3.0E−05 | 432 |
| 18 | 3773069 | 3773731 | promoter | ENST00000584060 | RP11-874J12.3 | −2.08 | 3.4E−08 | 662 |
| 1 | 65210283 | 65210996 | promoter | ENST00000371072 | RAVER2 | −1.16 | 7.6E−05 | 713 |
| 4 | 22970924 | 22971638 | intergenic | ENST00000511453 | RP11-412P11.1 | −1.27 | 2.9E−07 | 714 |
| 15 | 29966880 | 29967293 | promoter | ENST00000536835 | RP11-680F8.1 | −1.31 | 5.6E−05 | 413 |
| 8 | 4195706 | 4196553 | intron | ENST00000539096 | CSMD1 | −2.07 | 1.4E−09 | 847 |
| 18 | 21594009 | 21595594 | promoter | ENST00000579713 | RP11-403A21.2 | −1.10 | 8.6E−05 | 1585 |
| 18 | 13823915 | 13824237 | promoter | ENST00000390194 | AP001525.1 | −1.11 | 8.9E−05 | 322 |
| 17 | 48845654 | 48846094 | promoter | ENST00000502517 | LINC00483 | −1.26 | 3.3E−04 | 440 |
| 8 | 22601135 | 22601604 | promoter | ENST00000519624 | RP11-459E5.1 | −1.06 | 1.5E−04 | 469 |
| X | 19352288 | 19352590 | intergenic | ENST00000379806 | PDHA1 | −1.30 | 5.2E−04 | 302 |
| 14 | 65346358 | 65347344 | promoter | ENST00000542895 | SPTB | −1.01 | 2.5E−04 | 986 |
| 15 | 64540179 | 64540503 | intron | ENST00000606793 | CTD-2116N17.1 | −1.00 | 3.2E−04 | 324 |
| 6 | 82547755 | 82548150 | intergenic | ENST00000418567 | RP11-379B8.1 | −1.18 | 2.1E−05 | 395 |
| 11 | 104322692 | 104323628 | intron | ENST00000536529 | RP11-886D15.1 | −1.28 | 9.9E−05 | 936 |
| 17 | 46342828 | 46343603 | intron | ENST00000581419 | SKAP1 | −1.10 | 2.3E−04 | 775 |
| 2 | 146971789 | 146972404 | intergenic | ENST00000413391 | RPL17P12 | −1.68 | 2.1E−08 | 615 |
| X | 24517071 | 24517405 | intron | ENST00000493226 | PDK3 | −1.04 | 8.8E−04 | 334 |
| 12 | 15323979 | 15324554 | intron | ENST00000393736 | RERG | −2.21 | 7.2E−14 | 575 |
| 14 | 73928913 | 73929398 | promoter | ENST00000561382 | RP1-240K6.3 | −1.13 | 4.9E−07 | 485 |
| 12 | 71556548 | 71557645 | intron | ENST00000549421 | TSPAN8 | −1.66 | 4.2E−05 | 1097 |
| 4 | 77625261 | 77626040 | intron | ENST00000486758 | SHROOM3 | −1.47 | 1.7E−09 | 779 |
| 14 | 53167381 | 53167871 | intergenic | ENST00000556039 | ERO1L | −1.06 | 1.8E−04 | 490 |
| 15 | 83349039 | 83349480 | promoter | ENST00000543938 | AP3B2 | −1.68 | 4.4E−05 | 441 |
| 18 | 28591355 | 28591777 | intron | ENST00000434452 | DSC3 | −1.70 | 3.6E−04 | 422 |
| 6 | 30226869 | 30227564 | promoter | ENST00000420110 | HLA-L | −1.04 | 4.9E−07 | 695 |
| 12 | 12550932 | 12551724 | intron | ENST00000298571 | LOH12CR1 | −1.06 | 7.7E−04 | 792 |
| 18 | 7926531 | 7927006 | intron | ENST00000400053 | PTPRM | −1.56 | 9.3E−07 | 475 |
| 5 | 156874176 | 156874688 | intron | ENST00000519499 | CTB-109A12.1 | −1.29 | 6.0E−05 | 512 |
| 4 | 105415971 | 105416679 | promoter | ENST00000466963 | CXXC4 | −1.12 | 2.8E−07 | 708 |
| 1 | 247526375 | 247526698 | intergenic | ENST00000478225 | ZNF496 | 1.35 | 3.8E−04 | 323 |
| 14 | 68205454 | 68206247 | intron | ENST00000394455 | ZFYVE26 | −1.09 | 1.8E−05 | 793 |
| 18 | 21977090 | 21978175 | promoter | ENST00000582618 | OSBPL1A | −1.13 | 5.8E−04 | 1085 |
| 5 | 31020930 | 31021844 | intergenic | ENST00000495944 | RPL19P11 | −1.60 | 3.8E−06 | 914 |
| 17 | 73597354 | 73597809 | promoter | ENST00000584323 | MYO15B | −1.01 | 3.9E−04 | 455 |
| 1 | 165614855 | 165615573 | promoter | ENST00000461759 | MGST3 | −1.15 | 2.6E−04 | 718 |
| 12 | 89466458 | 89467244 | intron | ENST00000549278 | RP11-13A1.3 | −1.99 | 6.4E−04 | 786 |
| 4 | 139120636 | 139121025 | intron | ENST00000509248 | SLC7A11 | −1.06 | 2.2E−04 | 389 |
| 8 | 103941579 | 103942473 | promoter | ENST00000517996 | KB-1507C5.2 | −1.07 | 8.4E−09 | 894 |
| 15 | 36469921 | 36470501 | intron | ENST00000561394 | RP11-184D12.1 | −1.53 | 9.4E−04 | 580 |
| 8 | 15397612 | 15398367 | promoter | ENST00000503731 | TUSC3 | −1.80 | 5.8E−05 | 755 |
| 7 | 98013278 | 98014497 | promoter | ENST00000398259 | RPS3AP26 | −1.20 | 2.7E−04 | 1219 |
| 18 | 3051740 | 3052729 | intergenic | ENST00000356443 | MYOM1 | −1.45 | 3.8E−05 | 989 |
| 15 | 98491142 | 98491429 | intron | ENST00000538249 | ARRDC4 | −1.05 | 4.7E−04 | 287 |
| X | 24167349 | 24168808 | promoter | ENST00000427551 | ZFX-AS1 | −1.12 | 9.2E−07 | 1459 |
| 13 | 30682897 | 30683442 | promoter | ENST00000432770 | LINC00365 | −1.02 | 6.8E−04 | 545 |
| 10 | 65479858 | 65480099 | intron | ENST00000444770 | RP11-170M17.1 | −1.28 | 2.0E−06 | 241 |
| 22 | 43336262 | 43336736 | intron | ENST00000453079 | PACSIN2 | −1.07 | 4.8E−04 | 474 |
| 18 | 24235854 | 24237453 | promoter | ENST00000584630 | KCTD1 | −1.21 | 1.1E−04 | 1599 |
| 18 | 29665002 | 29665389 | intron | ENST00000583184 | RP11-53I6.2 | −1.47 | 4.1E−04 | 387 |
| X | 123540218 | 123540808 | intron | ENST00000469481 | STAG2 | −1.15 | 2.0E−06 | 590 |
| 21 | 29628568 | 29629059 | intergenic | ENST00000453420 | AL035610.2 | −1.35 | 1.8E−05 | 491 |
| 14 | 24777038 | 24777597 | promoter | ENST00000554411 | CIDEB | −1.09 | 5.1E−06 | 559 |
| 7 | 90350197 | 90350681 | intron | ENST00000436577 | CDK14 | −1.28 | 5.1E−05 | 484 |
| 3 | 118930104 | 118930466 | intergenic | ENST00000483209 | B4GALT4 | −1.33 | 9.1E−04 | 362 |
| 17 | 33759489 | 33760107 | promoter | ENST00000304905 | SLFN12 | −1.36 | 3.0E−05 | 618 |
| 6 | 126265396 | 126265975 | intergenic | ENST00000229633 | HINT3 | −1.22 | 5.2E−05 | 579 |
| 18 | 8341512 | 8342175 | intron | ENST00000577827 | PTPRM | −1.38 | 7.6E−05 | 663 |
| 13 | 60586478 | 60586983 | promoter | ENST00000435636 | DIAPH3-AS1 | −1.16 | 2.0E−04 | 505 |
| 2 | 43232429 | 43233212 | promoter | ENST00000457457 | AC016735.1 | −1.36 | 2.3E−07 | 783 |
| 4 | 72052163 | 72052582 | promoter | ENST00000264485 | SLC4A4 | −1.53 | 1.8E−07 | 419 |
| 18 | 11005554 | 11005954 | intron | ENST00000582913 | PIEZO2 | −1.21 | 3.1E−05 | 400 |
| 6 | 52254401 | 52254862 | intron | ENST00000360726 | PAQR8 | −1.13 | 1.8E−05 | 461 |
| 16 | 1031471 | 1032054 | promoter | ENST00000565467 | RP11-161M6.2 | −1.39 | 3.3E−04 | 583 |
| 14 | 68987627 | 68988132 | intron | ENST00000478014 | RAD51B | −1.03 | 2.2E−06 | 505 |
| 4 | 38387157 | 38387752 | intron | ENST00000503465 | RP11-83C7.1 | −1.45 | 1.6E−12 | 595 |
| 12 | 18951259 | 18952375 | intergenic | ENST00000317658 | CAPZA3 | −1.17 | 8.8E−04 | 1116 |
| 8 | 74219833 | 74220352 | intron | ENST00000520894 | RP11-434I12.2 | −1.28 | 8.7E−04 | 519 |
| 11 | 134526444 | 134526989 | intergenic | ENST00000529417 | RP11-469N6.3 | 2.13 | 6.3E−08 | 545 |
| 10 | 108273148 | 108273531 | intergenic | ENST00000399415 | RP11-446H13.2 | −1.96 | 1.5E−04 | 383 |
| 2 | 165770474 | 165770888 | promoter | ENST00000483641 | SLC38A11 | −1.19 | 5.7E−05 | 414 |
| 9 | 28915264 | 28915864 | intergenic | ENST00000401120 | MIR873 | −1.80 | 1.7E−04 | 600 |
| 1 | 244231070 | 244231550 | intron | ENST00000598000 | AL590483.1 | −1.21 | 4.3E−04 | 480 |
| 4 | 24384043 | 24384371 | intergenic | ENST00000410330 | AC092846.1 | −1.10 | 5.1E−05 | 328 |
| 5 | 103398196 | 103398978 | intergenic | ENST00000514769 | RP11-138J23.1 | −1.19 | 9.4E−04 | 782 |
| 8 | 1878704 | 1879351 | intron | ENST00000522435 | ARHGEF10 | −1.31 | 3.5E−04 | 647 |
| 8 | 37159582 | 37160492 | intergenic | ENST00000518765 | RP11-527N22.1 | −1.01 | 2.5E−04 | 910 |
| 19 | 10859669 | 10860777 | intron | ENST00000586939 | DNM2 | −1.13 | 1.9E−04 | 1108 |
| 8 | 38124767 | 38125231 | promoter | ENST00000530193 | PPAPDC1B | −1.13 | 8.4E−05 | 464 |
| 14 | 100625737 | 100626234 | promoter | ENST00000553834 | DEGS2 | −1.04 | 8.6E−04 | 497 |
| 17 | 70514867 | 70515633 | intron | ENST00000580861 | LINC00511 | −1.10 | 1.8E−04 | 766 |
| 11 | 22213851 | 22215484 | promoter | ENST00000324559 | ANO5 | −1.10 | 5.0E−06 | 1633 |
| 11 | 91530137 | 91530591 | promoter | ENST00000581290 | RP11-201M22.1 | −1.11 | 8.9E−04 | 454 |
| 4 | 174112844 | 174113342 | intron | ENST00000512285 | GALNT7 | −1.32 | 1.1E−04 | 498 |
| 8 | 98861557 | 98862712 | intron | ENST00000521545 | LAPTM4B | −1.07 | 3.5E−04 | 1155 |
| 12 | 132401688 | 132401954 | promoter | ENST00000540647 | ULK1 | 1.88 | 3.5E−05 | 266 |
| 10 | 98623698 | 98624364 | intron | ENST00000371097 | LCOR | −1.04 | 8.8E−04 | 666 |
| 5 | 67497853 | 67498258 | intergenic | ENST00000520762 | RP11-404L6.2 | −1.34 | 1.8E−04 | 405 |
| 8 | 71115117 | 71115743 | intron | ENST00000518287 | NCOA2 | −1.46 | 1.6E−05 | 626 |
| 18 | 20695658 | 20696122 | intergenic | ENST00000400473 | CABLES1 | −1.22 | 4.5E−05 | 464 |
| 18 | 19577616 | 19577921 | promoter | ENST00000577673 | AC091043.1 | −1.35 | 1.3E−05 | 305 |
| 17 | 72746567 | 72746861 | promoter | ENST00000585285 | MIR3615 | −1.28 | 8.9E−05 | 294 |
| 18 | 19866602 | 19866925 | intergenic | ENST00000459476 | snoU13 | −1.38 | 3.8E−06 | 323 |
| 1 | 2688905 | 2690000 | intron | ENST00000401095 | TTC34 | 1.02 | 3.3E−04 | 1095 |
| 12 | 15842656 | 15843267 | intron | ENST00000544064 | EPS8 | −1.26 | 9.2E−07 | 611 |
| 5 | 54467950 | 54468191 | promoter | ENST00000516047 | MIR449C | −1.04 | 4.2E−06 | 241 |
| 12 | 19219371 | 19219904 | intergenic | ENST00000449390 | RPL7P6 | −1.53 | 1.4E−04 | 533 |
| 2 | 109002050 | 109002496 | intron | ENST00000409309 | SULT1C4 | −1.20 | 5.7E−04 | 446 |
| 4 | 40475810 | 40476436 | promoter | ENST00000507180 | RBM47 | −1.30 | 1.1E−05 | 626 |
| 4 | 115484596 | 115485293 | intergenic | ENST00000310836 | UGT8 | −1.13 | 1.7E−04 | 697 |
| 5 | 56731545 | 56732157 | intron | ENST00000506106 | CTD-2023N9.1 | −1.02 | 1.9E−04 | 612 |
| 5 | 98215879 | 98216617 | intron | ENST00000284049 | CHD1 | −1.03 | 8.2E−04 | 738 |
| 6 | 155649620 | 155650370 | intergenic | ENST00000475849 | TFB1M | −1.27 | 4.6E−04 | 750 |
| 8 | 23039576 | 23039972 | intergenic | ENST00000518308 | RP11-1149O23.2 | −1.07 | 8.2E−04 | 396 |
| 14 | 65409340 | 65409856 | promoter | ENST00000557323 | GPX2 | −1.13 | 1.8E−05 | 516 |
| 18 | 12659958 | 12660445 | promoter | ENST00000589405 | PSMG2 | −1.99 | 1.3E−06 | 487 |
| 16 | 57286027 | 57286608 | promoter | ENST00000564376 | RP11-407G23.3 | −1.45 | 1.3E−04 | 581 |
| 12 | 89900906 | 89901589 | intron | ENST00000546830 | POC1B | −1.67 | 2.2E−05 | 683 |
| 3 | 172635673 | 172636396 | intron | ENST00000351008 | SPATA16 | −1.28 | 3.0E−04 | 723 |
| 6 | 56263991 | 56264896 | intergenic | ENST00000370819 | COL21A1 | −1.30 | 2.7E−05 | 905 |
| 8 | 86459177 | 86459730 | intergenic | ENST00000520459 | RP11-317J10.4 | −1.05 | 8.6E−04 | 553 |
| 18 | 21699037 | 21699241 | promoter | ENST00000583782 | RP11-799B12.2 | −1.68 | 9.4E−07 | 204 |
| 8 | 4188712 | 4189987 | intron | ENST00000539096 | CSMD1 | −1.19 | 7.6E−05 | 1275 |
| 15 | 41324040 | 41324393 | intron | ENST00000558357 | INO80 | 3.06 | 1.2E−04 | 353 |
| 7 | 57265415 | 57265595 | promoter | ENST00000423752 | RP11-1217F2.13 | 2.76 | 6.9E−04 | 180 |
| 12 | 7055207 | 7055997 | promoter | ENST00000538318 | PTPN6 | −1.13 | 4.7E−05 | 790 |
| 1 | 73361638 | 73361801 | intron | ENST00000445976 | RP4-660H19.1 | 2.75 | 4.5E−04 | 163 |
| 2 | 15499821 | 15500945 | intron | ENST00000442506 | NBAS | 2.05 | 8.1E−04 | 1124 |
| 6 | 97944099 | 97944304 | intergenic | ENST00000574739 | RP3-418C23.2 | 2.08 | 1.3E−04 | 205 |
| 19 | 31869090 | 31869843 | intron | ENST00000585336 | AC007796.1 | 1.50 | 9.7E−04 | 753 |
| 17 | 80544014 | 80544489 | promoter | ENST00000575578 | FOXK2 | 1.24 | 2.5E−04 | 475 |
| 7 | 148469337 | 148470194 | intron | ENST00000325222 | CUL1 | 1.23 | 9.7E−04 | 857 |
| 10 | 129595626 | 129595975 | intergenic | ENST00000388920 | FOXI2 | 1.21 | 7.1E−04 | 349 |
| 2 | 217237783 | 217238658 | promoter | ENST00000273067 | 4-Mar | 1.66 | 2.3E−04 | 875 |
| 19 | 38489929 | 38490545 | intron | ENST00000476317 | SIPA1L3 | 2.18 | 4.3E−06 | 616 |
| 10 | 133797280 | 133797729 | promoter | ENST00000368636 | BNIP3 | 1.38 | 2.4E−04 | 449 |
| 10 | 133661124 | 133661318 | intergenic | ENST00000341866 | AL450307.1 | 1.96 | 9.7E−04 | 194 |
| 2 | 36129295 | 36129643 | intergenic | ENST00000431951 | MRPL50P1 | 1.77 | 6.1E−04 | 348 |
| 4 | 122791099 | 122792004 | promoter | ENST00000567769 | RP11-63B13.1 | −1.05 | 3.2E−04 | 905 |
| 10 | 96989136 | 96989837 | promoter | ENST00000451737 | RP11-310E22.4 | 1.48 | 3.7E−04 | 701 |
| 12 | 6387233 | 6388200 | intergenic | ENST00000539998 | RP1-96H9.5 | −1.01 | 1.6E−04 | 967 |
| 1 | 237963084 | 237963484 | promoter | ENST00000466626 | RYR2 | −1.15 | 6.8E−04 | 400 |
| 11 | 117109912 | 117110426 | exon | ENST00000529869_361297 | | 1.45 | 8.5E−04 | 514 |
| 9 | 137494257 | 137495098 | intergenic | ENST00000371817 | COL5A1 | 1.68 | 1.7E−04 | 841 |
| 19 | 35809800 | 35810562 | promoter | ENST00000601414 | CD22 | 1.10 | 1.0E−04 | 762 |
| 19 | 38530496 | 38531253 | intron | ENST00000476317 | SIPA1L3 | 2.28 | 9.2E−07 | 757 |
| 12 | 108876411 | 108877044 | intron | ENST00000502160 | RP11-13G14.4 | 1.73 | 3.5E−05 | 633 |
| 1 | 210612139 | 210613054 | promoter | ENST00000367009 | HHAT | 1.58 | 2.2E−04 | 915 |
| 7 | 157599753 | 157600564 | intron | ENST00000404321 | PTPRN2 | 1.35 | 2.6E−04 | 811 |
| 17 | 68185179 | 68185450 | intergenic | ENST00000243457 | KCNJ2 | 1.93 | 3.5E−04 | 271 |
| 19 | 30019124 | 30019835 | promoter | ENST00000579268 | CTC-525D6.2 | 1.50 | 6.3E−04 | 711 |
| 7 | 154861699 | 154862044 | promoter | ENST00000287907 | HTR5A | 1.23 | 1.5E−04 | 345 |
| 7 | 2915618 | 2916223 | intergenic | ENST00000396946 | CARD11 | 1.24 | 8.6E−04 | 605 |
| 3 | 168602522 | 168603249 | intergenic | ENST00000484765 | RP11-368I23.2 | 1.30 | 6.4E−04 | 727 |
| 2 | 15309734 | 15310359 | intron | ENST00000485694 | NBAS | 1.55 | 1.8E−04 | 625 |
| 19 | 33367595 | 33368355 | promoter | ENST00000586628 | CTD-2085J24.4 | 1.70 | 7.0E−05 | 760 |
| 11 | 117151727 | 117152451 | promoter | ENST00000524917 | RNF214 | 1.29 | 6.4E−04 | 724 |
| 12 | 116400382 | 116401203 | promoter | ENST00000549725 | RP11-493P1.2 | 1.69 | 2.6E−05 | 821 |
| 4 | 85420209 | 85421036 | promoter | ENST00000295886 | NKX6-1 | −1.14 | 1.9E−04 | 827 |
| 19 | 37793700 | 37794465 | intergenic | ENST00000591471 | HKR1 | 1.62 | 3.6E−04 | 765 |
| 3 | 183894085 | 183894896 | promoter | ENST00000431779 | AP2M1 | 1.02 | 2.5E−04 | 811 |
| 16 | 86985326 | 86986094 | intergenic | ENST00000566109 | RP11-107C10.1 | 1.33 | 4.8E−04 | 768 |
| 3 | 14203211 | 14203401 | intron | ENST00000477324 | XPC | 2.20 | 3.0E−04 | 190 |
| 16 | 28394898 | 28395627 | intron | ENST00000398943 | EIF3CL | 1.66 | 3.5E−04 | 729 |
| 19 | 42617722 | 42618169 | intron | ENST00000531773 | POU2F2 | 1.24 | 8.3E−04 | 447 |
| 1 | 165868016 | 165868540 | promoter | ENST00000463772 | UCK2 | 1.08 | 8.5E−04 | 524 |
| 5 | 79715065 | 79715253 | intron | ENST00000510995 | ZFYVE16 | 2.32 | 2.0E−04 | 188 |
| X | 44731642 | 44733410 | promoter | ENST00000475233 | KDM6A | −1.02 | 7.2E−05 | 1768 |
| 19 | 36095937 | 36096410 | intergenic | ENST00000589603 | AC002115.9 | 1.37 | 9.3E−05 | 473 |
| 16 | 28742292 | 28743038 | promoter | ENST00000569005 | EIF3C | 1.36 | 6.4E−04 | 746 |
| 18 | 21032725 | 21033693 | promoter | ENST00000577501 | RIOK3 | −1.05 | 5.2E−04 | 968 |
| 11 | 12185010 | 12186343 | promoter | ENST00000379612 | MICAL2 | 1.03 | 9.2E−04 | 1333 |
| 14 | 76815171 | 76815651 | promoter | ENST00000390772 | AC016543.1 | 1.20 | 6.3E−04 | 480 |
| 17 | 21305235 | 21305901 | intron | ENST00000583088 | KCNJ12 | 1.03 | 7.6E−04 | 666 |
| 9 | 137394472 | 137395015 | intergenic | ENST00000444936 | RP11-473E2.2 | 1.21 | 7.7E−04 | 543 |
| 19 | 38704515 | 38705167 | promoter | ENST00000488378 | DPF1 | 1.71 | 5.1E−06 | 652 |
| 8 | 143273979 | 143275177 | intergenic | ENST00000517704 | LINC00051 | 1.44 | 3.1E−05 | 1198 |
| 18 | 24060728 | 24061749 | intron | ENST00000578973 | KCTD1 | −1.30 | 2.7E−04 | 1021 |
| 11 | 20118774 | 20119500 | intron | ENST00000311043 | NAV2 | 1.49 | 6.7E−04 | 726 |
| 14 | 56298766 | 56299226 | intergenic | ENST00000560336 | LINC00520 | −1.01 | 6.7E−05 | 460 |
| 20 | 22392204 | 22392708 | intron | ENST00000377121 | RP5-1004I9.1 | −1.09 | 5.9E−04 | 504 |
| 19 | 39564251 | 39564693 | intergenic | ENST00000601575 | PAPL | 1.36 | 8.2E−05 | 442 |
| 3 | 126326051 | 126326334 | promoter | ENST00000519162 | TXNRD3 | 1.54 | 5.5E−04 | 283 |
| 5 | 89316952 | 89317321 | intergenic | ENST00000584845 | MIR3660 | −1.03 | 4.1E−04 | 369 |
| 11 | 117069701 | 117070445 | promoter | ENST00000278968 | TAGLN | 1.19 | 4.0E−04 | 744 |
| 1 | 19586986 | 19587534 | intergenic | ENST00000330263 | MRTO4 | 1.32 | 5.2E−04 | 548 |
| 15 | 26020460 | 26021175 | intron | ENST00000555815 | ATP10A | 1.33 | 1.3E−06 | 715 |
| 2 | 102353912 | 102354557 | intron | ENST00000417294 | MAP4K4 | −1.29 | 7.3E−05 | 645 |
| 4 | 141264454 | 141264871 | promoter | ENST00000506322 | SCOC | −1.08 | 3.8E−05 | 417 |
| 2 | 242054831 | 242055272 | intron | ENST00000493544 | PASK | 1.77 | 1.7E−06 | 441 |
| 17 | 39686341 | 39686778 | promoter | ENST00000361566 | KRT19 | −1.12 | 1.3E−04 | 437 |
| 13 | 42270599 | 42271143 | promoter | ENST00000478987 | VWA8 | −1.26 | 3.1E−04 | 544 |
| 19 | 33236950 | 33238144 | intron | ENST00000421545 | TDRD12 | 1.04 | 4.6E−04 | 1194 |
| 12 | 33049306 | 33050344 | promoter | ENST00000546741 | PKP2 | −1.04 | 1.4E−04 | 1038 |
| 10 | 81239097 | 81239352 | intergenic | ENST00000557620 | TPRX1P1 | 1.62 | 4.5E−04 | 255 |
| 20 | 36919560 | 36920024 | exon | ENST00000451435_619426 | | 1.18 | 1.8E−04 | 464 |
| 10 | 126028465 | 126028958 | intergenic | ENST00000539214 | OAT | 1.54 | 9.5E−04 | 493 |
| 11 | 120088623 | 120089064 | intron | ENST00000531220 | OAF | 1.43 | 7.0E−04 | 441 |
| 15 | 51369174 | 51369713 | intron | ENST00000559909 | RP11-108K3.1 | 1.16 | 4.6E−04 | 539 |
| 16 | 19843028 | 19843331 | intron | ENST00000568061 | IQCK | 1.29 | 9.5E−04 | 303 |
| X | 1510891 | 1512012 | promoter | ENST00000484026 | SLC25A6 | −1.04 | 8.6E−04 | 1121 |
| 3 | 71591682 | 71592117 | promoter | ENST00000408337 | MIR1284 | 1.17 | 1.5E−04 | 435 |
| 19 | 33963942 | 33964303 | intron | ENST00000590408 | PEPD | 1.31 | 3.3E−04 | 361 |
| 17 | 64536177 | 64536808 | intron | ENST00000284384 | PRKCA | 1.39 | 9.4E−04 | 631 |
| 11 | 1078428 | 1079839 | intron | ENST00000359061 | MUC2 | 1.34 | 8.4E−04 | 1411 |
| 12 | 98793216 | 98793758 | intergenic | ENST00000364426 | RNU4-41P | 1.16 | 2.4E−05 | 542 |
| 1 | 15322511 | 15323031 | intron | ENST00000400797 | KAZN | 1.15 | 4.6E−04 | 520 |
| 2 | 208352490 | 208352976 | intron | ENST00000418850 | AC007879.5 | 1.98 | 2.9E−04 | 486 |
| 3 | 128914473 | 128915151 | intergenic | ENST00000422453 | CNBP | 1.16 | 4.6E−04 | 678 |
| 6 | 110064994 | 110065287 | intron | ENST00000230124 | FIG4 | 1.24 | 5.5E−04 | 293 |
| 7 | 86688557 | 86689480 | promoter | ENST00000423294 | KIAA1324L | −1.09 | 2.5E−04 | 923 |
| 3 | 127453590 | 127454743 | promoter | ENST00000398101 | MGLL | 1.22 | 1.0E−04 | 1153 |
| 9 | 127105090 | 127105743 | intron | ENST00000539416 | NEK6 | 1.31 | 1.7E−04 | 653 |
| 4 | 99064059 | 99065056 | promoter | ENST00000295268 | STPG2 | −1.10 | 9.8E−07 | 997 |
| 11 | 70496478 | 70496740 | intron | ENST00000445654 | SHANK2 | 1.30 | 6.5E−06 | 262 |
| 11 | 1691687 | 1692395 | intergenic | ENST00000382167 | FAM99A | 1.49 | 4.4E−05 | 708 |
| 4 | 173647115 | 173647791 | intron | ENST00000508122 | GALNTL6 | −1.60 | 1.3E−05 | 676 |
| 14 | 102172379 | 102172956 | intron | ENST00000557778 | RP11-1029J19.5 | 1.02 | 4.1E−04 | 577 |
| 18 | 21082967 | 21083951 | promoter | ENST00000592119 | C18orf8 | −1.12 | 9.3E−05 | 984 |
| 7 | 150810759 | 150811221 | promoter | ENST00000335367 | AGAP3 | 1.13 | 4.0E−04 | 462 |
| 2 | 74010590 | 74010935 | promoter | ENST00000409561 | C2orf78 | 1.10 | 3.0E−04 | 345 |
| 10 | 133759398 | 133760269 | intron | ENST00000472664 | PPP2R2D | 1.38 | 2.4E−04 | 871 |
| 8 | 101635463 | 101636150 | intron | ENST00000520661 | SNX31 | 1.30 | 9.0E−05 | 687 |
| 13 | 114579128 | 114579433 | promoter | ENST00000449453 | RP11-199F6.4 | 1.33 | 2.3E−04 | 305 |
| 12 | 47488676 | 47488915 | intron | ENST00000546455 | PCED1B | 1.53 | 9.5E−04 | 239 |
| 4 | 48946273 | 48946960 | intergenic | ENST00000507399 | RP11-317G22.2 | −1.22 | 2.1E−05 | 687 |
| 17 | 40074968 | 40075633 | promoter | ENST00000590735 | ACLY | −1.00 | 4.9E−04 | 665 |
| X | 16804037 | 16805127 | promoter | ENST00000398155 | TXLNG | −1.12 | 2.1E−05 | 1090 |
| 15 | 102215274 | 102215634 | intron | ENST00000539112 | TARSL2 | 1.43 | 6.6E−04 | 360 |
| 16 | 88840365 | 88840766 | intron | ENST00000301015 | PIEZO1 | 1.47 | 1.5E−04 | 401 |
| 2 | 239835989 | 239836732 | intergenic | ENST00000455228 | AC114788.2 | 1.19 | 5.2E−04 | 743 |
| 2 | 129063639 | 129064276 | intron | ENST00000494089 | HS6ST1 | 1.06 | 6.7E−04 | 637 |
| 1 | 230994632 | 230995105 | intron | ENST00000522201 | C1orf198 | 1.59 | 4.5E−04 | 473 |
| 1 | 12100647 | 12101031 | intergenic | ENST00000496974 | RN7SL649P | 1.01 | 7.1E−04 | 384 |
| 1 | 178877654 | 178877828 | intron | ENST00000478871 | RALGPS2 | 1.66 | 8.4E−04 | 174 |
| 17 | 15917197 | 15917706 | intron | ENST00000497842 | TTC19 | 1.20 | 7.6E−04 | 509 |
| 8 | 142157841 | 142158130 | intron | ENST00000523015 | DENND3 | 1.62 | 1.9E−04 | 289 |
| 10 | 121010086 | 121010469 | intron | ENST00000392870 | GRK5 | 1.31 | 2.5E−04 | 383 |
| 7 | 63212550 | 63212945 | intergenic | ENST00000605464 | CICP24 | 1.43 | 3.5E−05 | 395 |
| 12 | 131851320 | 131852149 | promoter | ENST00000539209 | RP13-507P19.1 | 1.52 | 5.6E−05 | 829 |
| 7 | 63217941 | 63218533 | intergenic | ENST00000605464 | CICP24 | 1.48 | 5.1E−05 | 592 |
| 5 | 40679080 | 40680306 | promoter | ENST00000514343 | PTGER4 | −1.00 | 5.4E−07 | 1226 |
| 7 | 155199524 | 155200087 | intergenic | ENST00000569431 | RP5-912I13.1 | 1.59 | 6.7E−06 | 563 |
| 5 | 628422 | 629006 | intron | ENST00000444221 | CEP72 | 1.27 | 1.7E−04 | 584 |
| 17 | 81140434 | 81141322 | intergenic | ENST00000572343 | AC139099.4 | 1.21 | 6.6E−04 | 888 |
| 7 | 63216118 | 63216460 | intergenic | ENST00000605464 | CICP24 | 1.48 | 1.8E−04 | 342 |
| 17 | 105730 | 106265 | intron | ENST00000570638 | RPH3AL | 1.38 | 1.2E−04 | 535 |
| 16 | 86878909 | 86879904 | intergenic | ENST00000566109 | RP11-107C10.1 | 1.25 | 3.6E−05 | 995 |
| 21 | 33157360 | 33157791 | intergenic | ENST00000610276 | AP000255.6 | 1.15 | 1.2E−04 | 431 |
| 11 | 22174396 | 22174976 | intergenic | ENST00000530837 | CTD-2019O4.1 | −1.71 | 4.2E−08 | 580 |
| 16 | 33293693 | 33295127 | intergenic | ENST00000573021 | RP11-23E10.5 | 1.15 | 8.0E−04 | 1434 |
| 5 | 2490324 | 2490714 | intergenic | ENST00000560688 | RP11-129I19.2 | 1.13 | 1.5E−04 | 390 |
| 19 | 1164280 | 1165046 | intron | ENST00000587655 | SBNO2 | 1.01 | 5.2E−04 | 766 |
| 13 | 113680424 | 113680653 | promoter | ENST00000473345 | MCF2L | 1.61 | 1.6E−04 | 229 |
| 14 | 60043166 | 60043680 | promoter | ENST00000281581 | CCDC175 | −1.25 | 1.8E−04 | 514 |
| 18 | 34408158 | 34409506 | promoter | ENST00000587139 | KIAA1328 | −1.07 | 1.1E−05 | 1348 |
| 17 | 55740045 | 55740953 | intron | ENST00000579505 | MSI2 | −1.01 | 1.1E−05 | 908 |
| 17 | 44438927 | 44439708 | promoter | ENST00000450673 | ARL17B | −1.22 | 6.7E−04 | 781 |
| 7 | 206405 | 206816 | intron | ENST00000477004 | FAM20C | 1.19 | 6.5E−04 | 411 |
| 7 | 63222975 | 63223858 | intergenic | ENST00000605464 | CICP24 | 1.12 | 5.0E−04 | 883 |
| 13 | 80055053 | 80055742 | promoter | ENST00000457171 | NDFIP2-AS1 | −1.00 | 4.2E−04 | 689 |
| 4 | 40578882 | 40579574 | intron | ENST00000513044 | RBM47 | −1.07 | 3.0E−08 | 692 |
| 9 | 140188004 | 140189043 | promoter | ENST00000566954 | RP13-122B23.8 | −1.23 | 8.9E−04 | 1039 |
| 17 | 70613945 | 70614728 | intron | ENST00000581549 | LINC00511 | −1.01 | 5.7E−06 | 783 |
| 5 | 74332978 | 74333338 | intergenic | ENST00000322348 | GCNT4 | −1.09 | 4.2E−04 | 360 |
| 4 | 1722559 | 1723411 | promoter | ENST00000536901 | TMEM129 | −1.03 | 1.4E−04 | 852 |
| 18 | 21166005 | 21167139 | promoter | ENST00000540608 | NPC1 | −1.36 | 1.1E−09 | 1134 |
| 17 | 39058236 | 39058611 | intergenic | ENST00000167588 | KRT20 | −1.19 | 5.7E−04 | 375 |
| 2 | 167231978 | 167233085 | promoter | ENST00000375387 | SCN9A | −1.12 | 1.6E−05 | 1107 |
| Y | 297421 | 298266 | intergenic | ENST00000516032 | RNU6-1334P | −1.02 | 5.9E−04 | 845 |
| 17 | 70462355 | 70462619 | intron | ENST00000580861 | LINC00511 | −1.03 | 4.9E−06 | 264 |
| 22 | 42709789 | 42710226 | intron | ENST00000515426 | TCF20 | −1.13 | 2.4E−04 | 437 |
| 13 | 30646504 | 30647236 | intergenic | ENST00000413591 | LINC00365 | −1.28 | 4.7E−05 | 732 |
| 18 | 77393621 | 77394083 | intergenic | ENST00000317008 | RP11-567M16.3 | 1.02 | 8.3E−04 | 462 |
| 17 | 73613416 | 73613713 | promoter | ENST00000578300 | MYO15B | −1.09 | 4.0E−05 | 297 |
| 18 | 20558174 | 20558672 | intron | ENST00000585177 | RBBP8 | −1.33 | 2.7E−07 | 498 |
| 21 | 18899540 | 18900000 | promoter | ENST00000363884 | Y_RNA | −1.31 | 8.0E−05 | 460 |
| 4 | 19557727 | 19558281 | intron | ENST00000511431 | RP11-608O21.1 | −1.74 | 1.6E−06 | 554 |
| 4 | 99582947 | 99583241 | exon | ENST00000569927_160528 | | −1.24 | 1.7E−04 | 294 |
| 15 | 102432818 | 102433991 | intergenic | ENST00000560907 | WBP1LP5 | 1.21 | 2.1E−04 | 1173 |
| 3 | 195487289 | 195487523 | intron | ENST00000480843 | MUC4 | 1.63 | 6.1E−05 | 234 |
| 19 | 2128409 | 2128837 | promoter | ENST00000590683 | AP3D1 | 2.03 | 4.6E−07 | 428 |
| 4 | 156679791 | 156681400 | promoter | ENST00000513437 | GUCY1B3 | −1.10 | 2.2E−06 | 1609 |
| 4 | 38735730 | 38736026 | intergenic | ENST00000410298 | RNA5SP158 | 1.17 | 5.5E−04 | 296 |
| X | 15755897 | 15756576 | promoter | ENST00000380319 | CA5B | −1.02 | 1.5E−04 | 679 |
| 19 | 51898699 | 51898961 | promoter | ENST00000600765 | CTD-2616J11.14 | 1.26 | 8.8E−04 | 262 |
| 4 | 103994568 | 103995223 | intron | ENST00000508136 | SLC9B2 | −1.52 | 4.2E−05 | 655 |
| 2 | 241564963 | 241565884 | promoter | ENST00000407714 | GPR35 | 1.21 | 1.8E−05 | 921 |
| 4 | 7404260 | 7404679 | intron | ENST00000329016 | SORCS2 | −1.76 | 1.7E−08 | 419 |
| 9 | 115851492 | 115852115 | intergenic | ENST00000439875 | FAM225B | 1.39 | 2.6E−04 | 623 |
| 17 | 79486482 | 79486780 | promoter | ENST00000442532 | RP13-766D20.2 | −1.29 | 2.9E−04 | 298 |
| 18 | 24159844 | 24160367 | intron | ENST00000580191 | KCTD1 | −1.80 | 3.4E−06 | 523 |
| 13 | 21277892 | 21278693 | promoter | ENST00000468605 | IL17D | −1.14 | 2.2E−05 | 801 |
| 18 | 2654993 | 2656229 | promoter | ENST00000579647 | CBX3P2 | −1.01 | 9.8E−04 | 1236 |
| 9 | 108081065 | 108081533 | intron | ENST00000607692 | SLC44A1 | −1.33 | 1.4E−04 | 468 |
| 10 | 35838253 | 35839249 | intron | ENST00000497692 | CCNY | 1.19 | 4.4E−04 | 996 |
| 18 | 3218007 | 3218215 | promoter | ENST00000261606 | MYOM1 | −1.32 | 6.7E−04 | 208 |
| 16 | 32351227 | 32353593 | intergenic | ENST00000562853 | RP11-17M15.4 | 1.20 | 6.5E−05 | 2366 |
| 4 | 41992323 | 41992873 | promoter | ENST00000510460 | SLC30A9 | −1.05 | 1.2E−05 | 550 |
| 4 | 122369404 | 122369799 | intergenic | ENST00000512282 | TUBB4BP5 | −1.10 | 4.1E−04 | 395 |
| 18 | 6315695 | 6316404 | intron | ENST00000580162 | L3MBTL4 | −2.14 | 3.8E−06 | 709 |
| 17 | 29816786 | 29817073 | promoter | ENST00000578694 | RAB11FIP4 | −1.46 | 1.2E−07 | 287 |
| 17 | 38501710 | 38502341 | promoter | ENST00000475125 | RARA | −1.14 | 5.8E−05 | 631 |
| 13 | 35923722 | 35924281 | intron | ENST00000379939 | NBEA | −1.45 | 9.2E−04 | 559 |
| 13 | 103553441 | 103553830 | intergenic | ENST00000605100 | METTL21EP | −1.72 | 8.0E−05 | 389 |
| 4 | 62406648 | 62407173 | intron | ENST00000514996 | LPHN3 | −1.51 | 4.9E−04 | 525 |
| 17 | 31281498 | 31281947 | intergenic | ENST00000578289 | TMEM98 | −1.10 | 8.1E−05 | 449 |
| 8 | 134440828 | 134441594 | intergenic | ENST00000393673 | ST13P6 | −1.33 | 3.1E−04 | 766 |
| 5 | 40784185 | 40784659 | intron | ENST00000397006 | PRKAA1 | −1.24 | 2.1E−04 | 474 |
| 4 | 185269668 | 185270393 | promoter | ENST00000511465 | RP11-290F5.2 | −1.12 | 1.4E−04 | 725 |
| 4 | 164471320 | 164471761 | intron | ENST00000510786 | 1-Mar | −1.12 | 1.3E−05 | 441 |
| 17 | 45393737 | 45394013 | intron | ENST00000575039 | RP11-290H9.4 | −1.59 | 1.3E−05 | 276 |
| 12 | 6873219 | 6873910 | promoter | ENST00000540667 | PTMS | −1.11 | 4.8E−04 | 691 |
| 1 | 201374557 | 201374865 | exon | ENST00000361379_57596 | | −1.02 | 2.7E−05 | 308 |
| 22 | 41983726 | 41984326 | promoter | ENST00000466645 | PMM1 | −1.17 | 8.3E−04 | 600 |
| 18 | 22067707 | 22067934 | promoter | ENST00000583122 | RP11-178F10.2 | −1.13 | 6.5E−04 | 227 |
| 17 | 74392058 | 74392341 | exon | ENST00000586409_558822 | | −1.21 | 2.0E−04 | 283 |
| 14 | 59296342 | 59296858 | promoter | ENST00000555378 | RP11-112J1.2 | −1.17 | 1.0E−04 | 516 |
| 4 | 103701581 | 103701969 | intergenic | ENST00000453744 | UBE2D3 | −1.03 | 5.8E−04 | 388 |
| 14 | 88715001 | 88715398 | intron | ENST00000556282 | KCNK10 | −1.14 | 3.7E−04 | 397 |
| 4 | 57107532 | 57108067 | intron | ENST00000264229 | KIAA1211 | −1.19 | 9.0E−04 | 535 |
| 18 | 52613423 | 52613785 | intron | ENST00000587148 | CCDC68 | −1.13 | 4.0E−05 | 362 |
| 4 | 129495033 | 129495556 | intergenic | ENST00000514265 | RP11-184M15.1 | −1.63 | 3.2E−06 | 523 |
| 10 | 112835917 | 112837154 | promoter | ENST00000280155 | ADRA2A | −1.34 | 9.1E−05 | 1237 |
| X | 7894985 | 7896017 | promoter | ENST00000442940 | PNPLA4 | −1.21 | 4.6E−08 | 1032 |
| 2 | 183956117 | 183956559 | intron | ENST00000444562 | AC064871.3 | −1.51 | 6.7E−04 | 442 |
| 18 | 71892391 | 71892807 | promoter | ENST00000480810 | RN7SL551P | −1.30 | 2.1E−04 | 416 |
| 6 | 2986172 | 2986575 | promoter | ENST00000450238 | LINC01011 | −1.22 | 1.0E−04 | 403 |
| 14 | 38438045 | 38438416 | intron | ENST00000533625 | TTC6 | −1.49 | 6.0E−04 | 371 |
| 4 | 30964479 | 30964886 | intron | ENST00000509759 | PCDH7 | −1.41 | 5.2E−05 | 407 |
| 18 | 29740444 | 29740915 | intron | ENST00000583696 | GAREM | −1.99 | 5.7E−05 | 471 |
| 17 | 57069125 | 57069558 | intron | ENST00000393066 | TRIM37 | −1.41 | 1.9E−07 | 433 |
| 12 | 105711706 | 105711997 | intron | ENST00000549251 | RP11-474B16.1 | −1.49 | 3.2E−04 | 291 |
| 18 | 20284179 | 20284604 | intron | ENST00000578831 | RP11-739L10.1 | −1.18 | 4.2E−05 | 425 |
| 17 | 64382980 | 64383423 | intron | ENST00000284384 | PRKCA | −1.24 | 1.5E−05 | 443 |
| 3 | 24640233 | 24640703 | intergenic | ENST00000415266 | EIF3KP2 | −1.32 | 4.0E−04 | 470 |
| 18 | 14430668 | 14431655 | intergenic | ENST00000584783 | LONRF2P1 | −1.04 | 9.7E−04 | 987 |
| 9 | 79249252 | 79250114 | intron | ENST00000223609 | PRUNE2 | −1.81 | 4.8E−08 | 862 |
| 3 | 24565803 | 24566193 | intergenic | ENST00000580344 | MIR4792 | −1.24 | 6.9E−04 | 390 |
| 4 | 108729691 | 108730105 | intergenic | ENST00000506462 | SGMS2 | −1.23 | 3.2E−04 | 414 |
| 12 | 3982194 | 3982816 | promoter | ENST00000450737 | PARP11 | −1.00 | 3.1E−04 | 622 |
| 14 | 50453931 | 50454479 | intron | ENST00000530176 | C14orf182 | −1.02 | 2.7E−04 | 548 |
| 2 | 42422735 | 42423150 | intron | ENST00000401738 | EML4 | −1.32 | 2.6E−04 | 415 |
| 8 | 8547367 | 8547711 | intergenic | ENST00000519106 | CLDN23 | −1.14 | 1.6E−04 | 344 |
| 1 | 28648608 | 28649153 | intergenic | ENST00000479574 | MED18 | −2.06 | 1.0E−07 | 545 |
| 12 | 646923 | 647267 | intron | ENST00000535680 | B4GALNT3 | −1.24 | 4.0E−04 | 344 |
| 8 | 22222876 | 22223300 | promoter | ENST00000359741 | SLC39A14 | −1.28 | 6.6E−04 | 424 |
| 5 | 162110217 | 162110778 | intergenic | ENST00000517722 | RP11-167P20.1 | −1.85 | 3.0E−04 | 561 |
| 22 | 50228082 | 50228576 | intron | ENST00000565177 | RP3-522J7.6 | −1.18 | 1.8E−04 | 494 |
| 12 | 1779737 | 1779986 | intergenic | ENST00000577921 | MIR3649 | −1.41 | 3.7E−04 | 249 |
| 13 | 24758417 | 24758918 | intron | ENST00000382141 | RP11-307N16.6 | −1.20 | 1.4E−04 | 501 |
| 4 | 187027154 | 187027446 | promoter | ENST00000508379 | FAM149A | −1.07 | 8.8E−05 | 292 |
| 4 | 149908119 | 149908467 | intergenic | ENST00000458836 | RNU7-197P | −1.05 | 8.7E−04 | 348 |
| 9 | 90184915 | 90185347 | intron | ENST00000489291 | DAPK1 | −1.44 | 3.3E−04 | 432 |
| 4 | 154140059 | 154140489 | intron | ENST00000338700 | TRIM2 | −1.62 | 9.8E−07 | 430 |
| 12 | 12556572 | 12557059 | intron | ENST00000298571 | LOH12CR1 | −1.35 | 3.7E−04 | 487 |
| 4 | 37684752 | 37685026 | intron | ENST00000454158 | RELL1 | −1.32 | 2.2E−04 | 274 |
| 17 | 62700725 | 62701052 | intergenic | ENST00000604003 | MINOS1P2 | −1.42 | 8.0E−05 | 327 |
| 17 | 79823676 | 79823948 | promoter | ENST00000576021 | RP11-498C9.3 | −1.24 | 6.2E−04 | 272 |
| 17 | 30533043 | 30533564 | promoter | ENST00000581148 | RHOT1 | −1.01 | 5.9E−04 | 521 |
| 18 | 25185269 | 25185490 | intergenic | ENST00000584546 | RP11-739N10.1 | −1.24 | 4.9E−04 | 221 |
| 18 | 2939329 | 2939618 | intron | ENST00000261596 | LPIN2 | −1.42 | 9.6E−05 | 289 |
| 18 | 19774213 | 19774529 | intron | ENST00000581694 | GATA6 | −1.46 | 9.2E−07 | 316 |
| 18 | 54937345 | 54938049 | intergenic | ENST00000365370 | RNU6-737P | −1.63 | 1.9E−04 | 704 |
| 3 | 191194228 | 191194546 | intergenic | ENST00000518817 | PYDC2 | −1.75 | 2.5E−05 | 318 |
| 5 | 90184384 | 90184958 | intron | ENST00000425867 | GPR98 | −1.05 | 2.7E−04 | 574 |
| 6 | 143160084 | 143160736 | promoter | ENST00000367604 | HIVEP2 | −1.11 | 4.9E−04 | 652 |
| 18 | 30050445 | 30051372 | promoter | ENST00000399218 | GAREM | −1.53 | 2.7E−04 | 927 |
| 3 | 43255202 | 43255564 | intergenic | ENST00000410399 | AC104434.1 | −1.35 | 1.8E−04 | 362 |
| 5 | 98360931 | 98361324 | intergenic | ENST00000513175 | CTD-2007H13.3 | −1.26 | 4.8E−06 | 393 |
| 19 | 45198585 | 45199263 | intron | ENST00000590796 | CTB-171A8.1 | −1.39 | 9.9E−05 | 678 |
| 17 | 76334969 | 76335254 | intron | ENST00000586321 | AC061992.2 | −1.35 | 2.2E−06 | 285 |
| 3 | 24358451 | 24358695 | intron | ENST00000418247 | THRB | −1.11 | 3.7E−04 | 244 |
| 4 | 31148080 | 31148352 | exon | ENST00000511884_155940 | | −1.70 | 1.2E−10 | 272 |
| 5 | 34212911 | 34213718 | intron | ENST00000512782 | RP11-1023L17.1 | −1.68 | 2.1E−05 | 807 |
| 10 | 482220 | 483506 | promoter | ENST00000425723 | RP11-490E15.2 | 2.06 | 9.3E−04 | 1286 |
| 12 | 132060998 | 132062024 | intergenic | ENST00000541343 | RP11-292117.1 | 2.01 | 2.0E−05 | 1026 |
| 20 | 61695692 | 61696532 | intron | ENST00000607802 | RP11-305P22.9 | 1.85 | 1.3E−04 | 840 |
| 4 | 7541341 | 7542231 | intron | ENST00000329016 | SORCS2 | 1.81 | 1.3E−04 | 890 |
| 16 | 88366497 | 88367260 | intergenic | ENST00000563190 | LA16c-444G7.1 | 1.32 | 2.6E−04 | 763 |
| 1 | 30664002 | 30664591 | intergenic | ENST00000442774 | RP3-357I16.1 | 1.41 | 2.7E−04 | 589 |
| 16 | 84558648 | 84558989 | intron | ENST00000565079 | TLDC1 | 1.67 | 6.6E−04 | 341 |
| 18 | 21453249 | 21453428 | promoter | ENST00000587184 | LAMA3 | −1.37 | 2.1E−05 | 179 |
| 3 | 195542062 | 195542854 | intergenic | ENST00000463781 | MUC4 | 1.03 | 9.1E−04 | 792 |
| 15 | 29269492 | 29270164 | promoter | ENST00000560531 | RP13-126C7.1 | 1.12 | 4.0E−05 | 672 |
| 8 | 143026250 | 143026924 | promoter | ENST00000408196 | AC104417.1 | 1.21 | 6.0E−04 | 674 |
| 2 | 233755631 | 233756268 | promoter | ENST00000461944 | NGEF | 1.49 | 2.4E−04 | 637 |
| X | 130712602 | 130713291 | intergenic | ENST00000444577 | OR13K1P | 1.94 | 4.5E−05 | 689 |
| 2 | 242838585 | 242839046 | intron | ENST00000429947 | AC131097.3 | 1.12 | 1.5E−04 | 461 |
| 19 | 38943593 | 38944148 | intron | ENST00000359596 | RYR1 | 1.41 | 7.3E−05 | 555 |
| 19 | 50215579 | 50216042 | promoter | ENST00000598072 | CPT1C | 1.61 | 4.9E−04 | 463 |
| 10 | 132897016 | 132897650 | intron | ENST00000368642 | TCERG1L | 1.35 | 1.9E−04 | 634 |
| 18 | 3117490 | 3118235 | intron | ENST00000261606 | MYOM1 | −1.48 | 4.7E−04 | 745 |
| 16 | 10394727 | 10395216 | intergenic | ENST00000564797 | ATF7IP2 | 1.47 | 3.0E−04 | 489 |
| 19 | 34112310 | 34112461 | promoter | ENST00000591231 | CHST8 | 1.65 | 6.3E−05 | 151 |
| 11 | 45149239 | 45150097 | intron | ENST00000530656 | PRDM11 | 1.11 | 4.1E−04 | 858 |
| 2 | 60524652 | 60525178 | intergenic | ENST00000457668 | AC007381.3 | 1.23 | 8.5E−04 | 526 |
| 18 | 19770500 | 19771301 | intron | ENST00000581694 | GATA6 | −1.05 | 9.9E−05 | 801 |
| 2 | 3497474 | 3498028 | intergenic | ENST00000607415 | RP11-1293J14.1 | 1.49 | 8.0E−05 | 554 |
| 20 | 55201436 | 55201906 | intergenic | ENST00000201031 | TFAP2C | 1.68 | 1.9E−04 | 470 |
| 19 | 39569172 | 39569875 | intergenic | ENST00000601575 | PAPL | 1.42 | 2.7E−04 | 703 |
| 19 | 51893704 | 51894598 | promoter | ENST00000570516 | C19orf84 | 1.52 | 2.2E−04 | 894 |
| 10 | 133908226 | 133908803 | intergenic | ENST00000298622 | JAKMIP3 | 1.61 | 5.7E−06 | 577 |
| 17 | 44656868 | 44657529 | promoter | ENST00000336125 | ARL17A | −1.17 | 6.7E−04 | 661 |
| 7 | 101321102 | 101321282 | intergenic | ENST00000223167 | MYL10 | 1.54 | 5.7E−04 | 180 |
| 3 | 139289513 | 139290376 | intron | ENST00000381790 | RP11-319G6.1 | 1.22 | 6.6E−05 | 863 |
| 7 | 6116687 | 6117343 | intergenic | ENST00000436915 | AC004895.4 | 1.06 | 9.1E−05 | 656 |
| 1 | 117635514 | 117636236 | promoter | ENST00000492682 | TTF2 | 1.20 | 7.6E−04 | 722 |
| 12 | 132816724 | 132819336 | intron | ENST00000328957 | GALNT9 | 1.52 | 2.6E−04 | 2612 |
| 1 | 16005038 | 16005519 | intergenic | ENST00000606262 | RP4-680D5.9 | 1.45 | 1.1E−04 | 481 |
| 18 | 29522315 | 29523852 | promoter | ENST00000580420 | RP11-326K13.4 | −1.51 | 8.6E−04 | 1537 |
| 1 | 17574935 | 17575827 | promoter | ENST00000375460 | PADI3 | 1.34 | 5.8E−04 | 892 |
| 9 | 104053040 | 104053880 | intron | ENST00000463206 | LPPR1 | 1.34 | 9.7E−04 | 840 |
| 15 | 80164774 | 80165510 | intron | ENST00000494999 | ST20-MTHFS | 1.13 | 8.1E−05 | 736 |
| 20 | 44978838 | 44979690 | exon | ENST00000493599_627499 | | 1.19 | 5.6E−05 | 852 |
| 16 | 56641008 | 56641623 | promoter | ENST00000245185 | MT2A | 1.21 | 3.2E−04 | 615 |
| 1 | 61105637 | 61106487 | promoter | ENST00000439156 | RP11-776H12.1 | 1.76 | 3.9E−06 | 850 |
| 9 | 139240060 | 139240754 | intron | ENST00000354753 | GPSM1 | 1.04 | 3.3E−04 | 694 |
| 16 | 53453058 | 53453761 | intergenic | ENST00000567964 | RBL2 | 1.22 | 5.7E−04 | 703 |
| 1 | 19724621 | 19725289 | intron | ENST00000482808 | CAPZB | 1.45 | 1.7E−04 | 668 |
| 17 | 60266034 | 60266758 | intergenic | ENST00000577881 | RP11-51L5.3 | −1.21 | 7.8E−05 | 724 |
| 19 | 52645300 | 52645902 | promoter | ENST00000597886 | CTC-471J1.9 | 1.26 | 1.9E−04 | 602 |
| 11 | 33202571 | 33203188 | intron | ENST00000500025 | CSTF3-AS1 | 1.21 | 7.8E−04 | 617 |
| 14 | 81769514 | 81770277 | intron | ENST00000556280 | STON2 | −1.00 | 3.0E−06 | 763 |
| 11 | 9567258 | 9568184 | intergenic | ENST00000396602 | ZNF143 | 1.34 | 2.6E−04 | 926 |
| 5 | 34466571 | 34467442 | intergenic | ENST00000503549 | RP11-1325J9.1 | −1.32 | 1.0E−09 | 871 |
| 2 | 237573927 | 237574674 | intergenic | ENST00000455068 | AC011286.1 | 1.18 | 8.6E−04 | 747 |
| 7 | 114670431 | 114671261 | intergenic | ENST00000257724 | MDFIC | 1.48 | 6.6E−04 | 830 |
| 4 | 2420021 | 2420910 | promoter | ENST00000382849 | RP11-503N18.1 | −1.03 | 6.5E−04 | 889 |
| 3 | 80745459 | 80745848 | intergenic | ENST00000482003 | RP11-47P18.1 | −1.28 | 2.8E−06 | 389 |
| 4 | 125353676 | 125354469 | intergenic | ENST00000506481 | RP11-93I21.2 | −1.10 | 2.8E−06 | 793 |
| 2 | 165477406 | 165478493 | promoter | ENST00000446413 | GRB14 | −1.03 | 5.2E−04 | 1087 |
| 19 | 31899364 | 31900164 | intron | ENST00000585336 | AC007796.1 | 1.30 | 6.5E−04 | 800 |
| 20 | 45887465 | 45888265 | intron | ENST00000468376 | ZMYND8 | 1.10 | 4.6E−04 | 800 |
| 4 | 54342467 | 54343100 | intron | ENST00000507166 | FIP1L1 | 1.32 | 2.9E−04 | 633 |
| 1 | 25296870 | 25297681 | promoter | ENST00000568143 | RP11-84D1.2 | 1.02 | 6.3E−04 | 811 |
| X | 2815696 | 2816658 | intergenic | ENST00000381154 | ARSD | −1.22 | 2.8E−06 | 962 |
| 12 | 7950400 | 7950813 | intergenic | ENST00000229307 | NANOG | −1.42 | 1.7E−04 | 413 |
| 1 | 92791916 | 92792644 | intron | ENST00000610020 | RPAP2 | 1.20 | 5.9E−04 | 728 |
| 5 | 92414000 | 92415132 | intergenic | ENST00000515153 | CTD-2091N23.1 | −1.04 | 2.9E−06 | 1132 |
| 11 | 70270264 | 70270605 | promoter | ENST00000393747 | CTTN | 1.11 | 9.0E−04 | 341 |
| 18 | 24067372 | 24067793 | intron | ENST00000578973 | KCTD1 | −1.42 | 3.2E−04 | 421 |
| X | 15692727 | 15694099 | promoter | ENST00000380333 | CA5BP1 | −1.05 | 1.8E−05 | 1372 |
| 3 | 195890536 | 195890927 | intergenic | ENST00000457079 | LINC00885 | 1.15 | 4.3E−04 | 391 |
| 10 | 133849722 | 133850635 | intergenic | ENST00000368636 | BNIP3 | 1.05 | 6.1E−05 | 913 |
| 1 | 29839867 | 29840197 | intergenic | ENST00000515851 | RP11-810H18.1 | 1.02 | 8.7E−04 | 330 |
| 12 | 132280700 | 132281100 | promoter | ENST00000537582 | SFSWAP | 1.26 | 2.7E−04 | 400 |
| 4 | 120549649 | 120550511 | promoter | ENST00000354960 | PDE5A | −1.27 | 5.1E−07 | 862 |
| 5 | 60954962 | 60955315 | exon | ENST00000505623_198864 | | −1.11 | 8.6E−04 | 353 |
| 8 | 107630045 | 107630587 | promoter | ENST00000497705 | OXR1 | −1.06 | 4.7E−05 | 542 |
| 10 | 132892787 | 132893492 | promoter | ENST00000436942 | TCERG1L-AS1 | 1.17 | 2.2E−04 | 705 |
| 7 | 16961496 | 16961960 | intergenic | ENST00000419352 | AC098592.7 | −1.21 | 2.5E−04 | 464 |
| 8 | 142597388 | 142597870 | intergenic | ENST00000427937 | AC138647.1 | 1.15 | 4.2E−05 | 482 |
| 4 | 125127833 | 125128704 | intron | ENST00000507299 | CTD-2325B11.1 | −1.33 | 5.1E−05 | 871 |
| 2 | 233124653 | 233125150 | exon | ENST00000433430_85344 | | 1.04 | 4.0E−04 | 497 |
| 1 | 6305892 | 6306263 | promoter | ENST00000377898 | HES3 | 1.14 | 6.6E−04 | 371 |
| X | 47052740 | 47053352 | promoter | ENST00000335972 | UBA1 | −1.22 | 6.5E−06 | 612 |
| 20 | 59832756 | 59833009 | intron | ENST00000360469 | CDH4 | 1.72 | 5.8E−04 | 253 |
| 5 | 87564239 | 87565285 | promoter | ENST00000512724 | TMEM161B-AS1 | −1.06 | 5.2E−05 | 1046 |
| 4 | 124467237 | 124467606 | intergenic | ENST00000508291 | RP11-381N20.1 | −1.29 | 4.6E−05 | 369 |
| 2 | 241811517 | 241811995 | promoter | ENST00000476698 | AGXT | 1.47 | 1.3E−06 | 478 |
| 16 | 73116469 | 73116806 | intergenic | ENST00000569990 | HCCAT5 | 1.31 | 2.4E−04 | 337 |
| 16 | 32639949 | 32640460 | intergenic | ENST00000564327 | RP11-96K14.1 | 1.29 | 2.5E−04 | 511 |
| 7 | 151169967 | 151170459 | promoter | ENST00000482053 | RHEB | 1.27 | 6.5E−04 | 492 |
| 15 | 59548285 | 59548587 | intron | ENST00000558571 | MYO1E | −1.06 | 2.4E−04 | 302 |
| 16 | 63651192 | 63652144 | promoter | ENST00000563855 | RP11-368L12.1 | −1.25 | 5.9E−04 | 952 |
| 19 | 30154965 | 30155734 | promoter | ENST00000436066 | PLEKHF1 | 1.11 | 3.8E−06 | 769 |
| 7 | 5635384 | 5635656 | promoter | ENST00000405801 | FSCN1 | 1.01 | 7.3E−05 | 272 |
| 11 | 2008321 | 2008791 | intron | ENST00000419080 | MRPL23-AS1 | 1.26 | 3.8E−05 | 470 |
| 4 | 142271254 | 142271697 | intergenic | ENST00000511213 | RP11-362F19.1 | −1.05 | 4.3E−04 | 443 |
| X | 7050318 | 7051134 | intron | ENST00000498474 | HDHD1 | −1.08 | 1.8E−05 | 816 |
| 4 | 176986570 | 176987383 | promoter | ENST00000280190 | WDR17 | −1.55 | 5.1E−05 | 813 |
| 3 | 15900398 | 15901920 | promoter | ENST00000439830 | ANKRD28 | −1.06 | 4.2E−04 | 1522 |
| 18 | 21408398 | 21408763 | promoter | ENST00000591749 | LAMA3 | −1.25 | 1.7E−06 | 365 |
| 4 | 36352766 | 36353045 | intron | ENST00000504344 | RP11-431M7.2 | −1.08 | 1.8E−04 | 279 |
| 4 | 26828299 | 26828789 | intergenic | ENST00000494628 | STIM2 | −1.12 | 5.5E−04 | 490 |
| 19 | 34760796 | 34761482 | intron | ENST00000585833 | KIAA0355 | 1.04 | 1.6E−05 | 686 |
| 3 | 188506277 | 188507139 | intron | ENST00000459897 | LPP | 1.01 | 3.1E−04 | 862 |
| 17 | 36507408 | 36508157 | promoter | ENST00000577233 | SOCS7 | −1.02 | 2.2E−05 | 749 |
| 4 | 149297345 | 149297623 | intron | ENST00000511528 | NR3C2 | −1.19 | 5.8E−04 | 278 |
| 19 | 38538873 | 38540260 | intron | ENST00000476317 | SIPA1L3 | 1.19 | 3.3E−04 | 1387 |
| 12 | 17795043 | 17795272 | intergenic | ENST00000539105 | RP11-606D9.1 | −1.30 | 8.9E−06 | 229 |
| 11 | 64512396 | 64512888 | promoter | ENST00000377485 | RASGRP2 | 1.12 | 6.7E−04 | 492 |
| 18 | 77679919 | 77680340 | intron | ENST00000478144 | PQLC1 | 1.18 | 5.5E−04 | 421 |
| 5 | 156692779 | 156693779 | promoter | ENST00000517634 | CTC-248O19.1 | −1.15 | 8.9E−07 | 1000 |
| 19 | 38524195 | 38525390 | intron | ENST00000476317 | SIPA1L3 | 1.46 | 2.0E−04 | 1195 |
| 18 | 21452574 | 21453145 | promoter | ENST00000587184 | LAMA3 | −1.60 | 8.0E−11 | 571 |
| 19 | 36760064 | 36760513 | intergenic | ENST00000355114 | ZNF565 | 1.43 | 9.9E−04 | 449 |
| 4 | 90226929 | 90227192 | promoter | ENST00000609438 | GPRIN3 | −1.34 | 7.6E−05 | 263 |
| 16 | 4464103 | 4464762 | promoter | ENST00000576457 | CORO7 | −1.03 | 5.3E−04 | 659 |
| X | 24482963 | 24483767 | promoter | ENST00000441463 | PDK3 | −1.15 | 2.0E−06 | 804 |
| 18 | 12657581 | 12658532 | promoter | ENST00000400512 | AP005482.1 | −1.10 | 6.7E−04 | 951 |
| 7 | 534134 | 534368 | promoter | ENST00000434541 | AC147651.1 | 1.29 | 7.7E−07 | 234 |
| 7 | 30829073 | 30829346 | intron | ENST00000451002 | INMT-FAM188B | 1.25 | 4.4E−04 | 273 |
| 5 | 70743142 | 70743357 | promoter | ENST00000502659 | RP11-136K7.2 | −1.14 | 7.6E−04 | 215 |
| 3 | 195510841 | 195511431 | exon | ENST00000478156_152007 | | 1.25 | 9.4E−04 | 590 |
| 4 | 54457506 | 54458027 | promoter | ENST00000512247 | LNX1 | −1.07 | 9.4E−06 | 521 |
| 16 | 4394345 | 4394677 | promoter | ENST00000575848 | PAM16 | 1.18 | 4.0E−04 | 332 |
| 10 | 11927228 | 11927674 | intron | ENST00000445498 | PROSER2-AS1 | 1.49 | 3.1E−04 | 446 |
| 22 | 43892550 | 43892910 | intron | ENST00000538182 | MPPED1 | 1.12 | 3.3E−04 | 360 |
| 9 | 114827947 | 114828604 | intron | ENST00000374264 | SUSD1 | −1.39 | 1.0E−04 | 657 |
| 20 | 59950361 | 59951203 | intron | ENST00000360469 | CDH4 | 1.05 | 5.7E−04 | 842 |
| 17 | 72987700 | 72988299 | intron | ENST00000337231 | CDR2L | −1.01 | 2.0E−04 | 599 |
| 17 | 62161429 | 62162290 | intron | ENST00000584041 | ERN1 | −1.17 | 5.3E−05 | 861 |
| 18 | 20263110 | 20263735 | intergenic | ENST00000578831 | RP11-739L10.1 | −1.58 | 3.4E−08 | 625 |
| 20 | 31208975 | 31209164 | intergenic | ENST00000360785 | C20orf203 | 1.39 | 1.7E−04 | 189 |
| 7 | 158995289 | 158995591 | intergenic | ENST00000437005 | PIP5K1P2 | 1.27 | 5.3E−04 | 302 |
| 8 | 17658296 | 17659254 | promoter | ENST00000522768 | RP11-156K13.1 | −1.23 | 3.1E−04 | 958 |
| 19 | 1144620 | 1144966 | intron | ENST00000587655 | SBNO2 | 1.32 | 8.2E−04 | 346 |
| 2 | 97117403 | 97117850 | intergenic | ENST00000310865 | NEURL3 | 1.02 | 1.3E−04 | 447 |
| 1 | 245100328 | 245100603 | intergenic | ENST00000364888 | RN7SKP55 | 1.66 | 1.0E−04 | 275 |
| 19 | 38735536 | 38736387 | promoter | ENST00000590510 | SPINT2 | 1.49 | 2.8E−04 | 851 |
| 19 | 34809126 | 34810741 | intron | ENST00000588338 | KIAA0355 | 1.38 | 7.8E−06 | 1615 |
| 17 | 854896 | 856177 | intron | ENST00000575171 | NXN | 1.26 | 2.9E−04 | 1281 |
| 19 | 31830912 | 31831630 | intron | ENST00000558569 | TSHZ3 | 1.69 | 4.6E−04 | 718 |
| 19 | 38905395 | 38905919 | promoter | ENST00000588708 | RASGRP4 | 1.69 | 5.2E−05 | 524 |
| 13 | 30122775 | 30123280 | intron | ENST00000450494 | SLC7A1 | −1.31 | 2.2E−04 | 505 |
| 3 | 152974102 | 152975125 | intergenic | ENST00000582522 | RN7SL300P | 1.04 | 4.6E−04 | 1023 |
| 17 | 56494818 | 56495318 | promoter | ENST00000580014 | RNF43 | −1.02 | 1.8E−04 | 500 |
| 12 | 15427333 | 15427966 | intron | ENST00000393736 | RERG | −1.30 | 1.0E−05 | 633 |
| 18 | 19862218 | 19863030 | intergenic | ENST00000459476 | snoU13 | −1.39 | 6.3E−06 | 812 |
| 14 | 31697679 | 31698056 | intergenic | ENST00000365532 | Y_RNA | −1.23 | 7.5E−05 | 377 |
| 20 | 55363228 | 55363724 | intergenic | ENST00000384429 | RNU6-929P | 1.55 | 1.6E−06 | 496 |
| 19 | 36799597 | 36800084 | promoter | ENST00000600983 | CTD-3162L10.1 | 1.36 | 8.1E−04 | 487 |
| 19 | 31828906 | 31829306 | intron | ENST00000558569 | TSHZ3 | 1.46 | 3.1E−04 | 400 |
| 4 | 79548832 | 79549112 | intergenic | ENST00000364128 | Y_RNA | −1.04 | 8.0E−04 | 280 |
| 1 | 148929648 | 148931757 | promoter | ENST00000457390 | RP11-14N7.2 | 1.14 | 5.7E−04 | 2109 |
| 16 | 57298954 | 57299312 | promoter | ENST00000564018 | PLLP | −1.09 | 1.7E−07 | 358 |
| 18 | 20679542 | 20679947 | intergenic | ENST00000400473 | CABLES1 | −1.17 | 2.1E−05 | 405 |
| 12 | 12223581 | 12224233 | promoter | ENST00000308721 | BCL2L14 | −1.13 | 2.6E−04 | 652 |
| 5 | 170224689 | 170225199 | intron | ENST00000519598 | GABRP | 1.03 | 2.0E−04 | 510 |
| 8 | 118958604 | 118959299 | intron | ENST00000436216 | EXT1 | −1.00 | 6.4E−04 | 695 |
| 5 | 170184196 | 170184589 | promoter | ENST00000521965 | MIR4454 | −1.23 | 1.2E−05 | 393 |
| 15 | 39565852 | 39566905 | promoter | ENST00000561058 | RP11-624L4.1 | −1.14 | 1.8E−08 | 1053 |
| 5 | 81931049 | 81932003 | intergenic | ENST00000510845 | CTD-2015A6.2 | −1.01 | 2.7E−04 | 954 |
| 1 | 8800026 | 8800575 | intron | ENST00000480342 | RERE | 1.26 | 8.8E−04 | 549 |
| 14 | 87265459 | 87266198 | intergenic | ENST00000557527 | RP11-322L20.1 | −1.35 | 8.6E−04 | 739 |
| 4 | 169019178 | 169019931 | intron | ENST00000506926 | RP11-310I9.1 | −1.29 | 1.3E−06 | 753 |
| 1 | 165742556 | 165743015 | exon | ENST00000423121_23045 | | 1.33 | 7.8E−04 | 459 |
| 1 | 180126329 | 180127241 | intron | ENST00000367600 | QSOX1 | −1.08 | 4.7E−04 | 912 |
| 3 | 195627548 | 195627967 | intron | ENST00000468819 | TNK2 | 1.01 | 8.7E−04 | 419 |
| 1 | 68345690 | 68346295 | intron | ENST00000413628 | GNG12-AS1 | −1.11 | 1.1E−04 | 605 |
| 5 | 95429064 | 95430289 | intron | ENST00000511775 | CTD-2337A12.1 | −1.22 | 6.0E−05 | 1225 |
| 12 | 113342092 | 113342931 | promoter | ENST00000202917 | OAS1 | 1.04 | 9.7E−04 | 839 |
| 14 | 50908246 | 50909117 | intron | ENST00000013125 | MAP4K5 | −1.07 | 4.0E−05 | 871 |
| 16 | 56687942 | 56688603 | intergenic | ENST00000334346 | MT1B | 1.20 | 9.2E−04 | 661 |
| 4 | 98353586 | 98354125 | intron | ENST00000518105 | RP11-681L8.1 | −1.30 | 8.0E−05 | 539 |
| X | 17613238 | 17614124 | intron | ENST00000380060 | NHS | −1.26 | 3.9E−04 | 886 |
| 7 | 86475603 | 86476697 | intron | ENST00000439827 | GRM3 | −1.30 | 5.4E−04 | 1094 |
| 13 | 73745224 | 73745935 | intergenic | ENST00000364383 | RNU4-10P | −1.20 | 9.9E−05 | 711 |
| 13 | 39260761 | 39261550 | promoter | ENST00000280481 | FREM2 | −1.21 | 2.3E−10 | 789 |
| 1 | 11999122 | 11999719 | intron | ENST00000196061 | PLOD1 | 1.23 | 9.4E−04 | 597 |
| 18 | 14178703 | 14179225 | promoter | ENST00000581181 | ANKRD20A5P | −1.32 | 1.2E−05 | 522 |
| 6 | 15949256 | 15950233 | intergenic | ENST00000448802 | ARPC3P5 | −1.21 | 3.3E−04 | 977 |
| 5 | 73704005 | 73704568 | intron | ENST00000507781 | CTC-419K13.1 | −1.18 | 2.1E−05 | 563 |
| 4 | 184276391 | 184276972 | intergenic | ENST00000514910 | RP11-451F20.1 | −1.10 | 3.7E−04 | 581 |
| 18 | 29952163 | 29952949 | intron | ENST00000269209 | GAREM | −1.80 | 1.2E−05 | 786 |
| 4 | 120651110 | 120651691 | intergenic | ENST00000503266 | RP11-236P13.1 | −1.15 | 8.8E−06 | 581 |
| 4 | 147866860 | 147867427 | promoter | ENST00000502319 | TTC29 | −1.10 | 5.1E−05 | 567 |
| 13 | 24606606 | 24607289 | intron | ENST00000382141 | RP11-307N16.6 | −1.27 | 4.9E−06 | 683 |
| 9 | 116333099 | 116333705 | intron | ENST00000428429 | RP11-168K11.2 | −1.05 | 1.5E−04 | 606 |
| 16 | 52288281 | 52288983 | promoter | ENST00000408588 | AC007333.1 | −1.15 | 9.5E−04 | 702 |
| 4 | 168139291 | 168139787 | intron | ENST00000512042 | SPOCK3 | −1.35 | 6.0E−04 | 496 |
| 2 | 237791572 | 237792049 | intergenic | ENST00000413385 | AC011286.1 | 1.28 | 2.9E−04 | 477 |
| 1 | 4016604 | 4017089 | intergenic | ENST00000412674 | RP13-614K11.1 | 1.09 | 4.1E−04 | 485 |
| 5 | 50728721 | 50729673 | intergenic | ENST00000505723 | CTD-2335O3.2 | −1.01 | 1.4E−04 | 952 |
| 10 | 14862005 | 14862511 | intron | ENST00000465530 | CDNF | 1.30 | 9.5E−04 | 506 |
| 4 | 111751532 | 111751971 | intergenic | ENST00000515999 | AC024198.1 | −1.22 | 6.6E−04 | 439 |
| X | 64416588 | 64417229 | intergenic | ENST00000451184 | RP11-231N9.1 | −1.05 | 3.8E−04 | 641 |
| 1 | 227947119 | 227947769 | intron | ENST00000478768 | SNAP47 | 1.30 | 6.6E−05 | 650 |
| 13 | 76583584 | 76584230 | intergenic | ENST00000448806 | LINC01034 | −1.58 | 7.1E−05 | 646 |
| 18 | 21207297 | 21207674 | intron | ENST00000587763 | ANKRD29 | −1.54 | 1.3E−08 | 377 |
| 22 | 32475114 | 32475693 | intron | ENST00000543737 | SLC5A1 | −1.52 | 4.1E−07 | 579 |
| 3 | 126678871 | 126679767 | intron | ENST00000510044 | CHCHD6 | 1.17 | 1.3E−06 | 896 |
| 4 | 106830892 | 106831539 | promoter | ENST00000506056 | NPNT | −1.21 | 7.1E−06 | 647 |
| 15 | 63343399 | 63343882 | promoter | ENST00000561241 | RP11-244F12.3 | −1.35 | 4.6E−05 | 483 |
| 3 | 141133388 | 141134001 | intron | ENST00000513570 | ZBTB38 | 1.28 | 9.2E−04 | 613 |
| 21 | 36391861 | 36392371 | intron | ENST00000416754 | RUNX1 | −1.14 | 4.7E−04 | 510 |
| 13 | 103782751 | 103783563 | intergenic | ENST00000245312 | SLC10A2 | −1.43 | 2.0E−05 | 812 |
| 5 | 110072468 | 110072845 | promoter | ENST00000512886 | TMEM232 | −1.09 | 1.4E−04 | 377 |
| 9 | 89951812 | 89952262 | intergenic | ENST00000391119 | SNORA26 | −1.23 | 7.0E−08 | 450 |
| 18 | 8794410 | 8794963 | intron | ENST00000518815 | SOGA2 | −1.63 | 6.2E−05 | 553 |
| 10 | 79115617 | 79115970 | promoter | ENST00000418515 | RP11-619F23.2 | 1.10 | 6.8E−04 | 353 |
| 17 | 48770069 | 48771000 | promoter | ENST00000574246 | RP11-294J22.6 | −1.21 | 9.2E−05 | 931 |
| 5 | 14581642 | 14582228 | promoter | ENST00000274217 | FAM105A | −1.12 | 2.4E−05 | 586 |
| 18 | 71007537 | 71008213 | intron | ENST00000583942 | CTD-2354A18.1 | −1.40 | 7.0E−04 | 676 |
| 22 | 34142384 | 34142996 | intron | ENST00000416275 | LARGE-AS1 | −1.32 | 4.7E−04 | 612 |
| 19 | 51596977 | 51597664 | intergenic | ENST00000421832 | CTU1 | 1.06 | 8.0E−04 | 687 |
| 18 | 7878650 | 7879298 | intron | ENST00000400053 | PTPRM | −1.20 | 1.8E−04 | 648 |
| 4 | 67440362 | 67441524 | intergenic | ENST00000470993 | RPS23P3 | −1.27 | 3.6E−08 | 1162 |
| 11 | 68847695 | 68848373 | intron | ENST00000442692 | TPCN2 | 1.11 | 3.5E−05 | 678 |
| 15 | 86106408 | 86107073 | intron | ENST00000558811 | AKAP13 | −1.08 | 1.4E−04 | 665 |
| 14 | 38063747 | 38065628 | promoter | ENST00000556845 | TTC6 | −1.08 | 4.5E−07 | 1881 |
| 13 | 74864507 | 74864895 | promoter | ENST00000383890 | RNY1P5 | −1.55 | 6.9E−04 | 388 |
| 22 | 40783623 | 40784186 | promoter | ENST00000607915 | RP5-1042K10.10 | −1.27 | 4.5E−04 | 563 |
| 18 | 23669906 | 23671402 | promoter | ENST00000578595 | SS18 | −1.27 | 4.1E−05 | 1496 |
| 2 | 228626684 | 228627219 | promoter | ENST00000516537 | RNA5SP121 | −1.67 | 2.7E−04 | 535 |
| 14 | 75749392 | 75750562 | intergenic | ENST00000303562 | FOS | −1.27 | 3.2E−06 | 1170 |
| 5 | 34717596 | 34718270 | promoter | ENST00000502736 | RAI14 | 1.03 | 1.4E−04 | 674 |
| 1 | 204616727 | 204616979 | intron | ENST00000496057 | LRRN2 | 1.14 | 1.1E−04 | 252 |
| 9 | 132105932 | 132106561 | promoter | ENST00000423122 | RP11-65J3.1 | 1.05 | 3.0E−04 | 629 |
| 19 | 7489776 | 7490370 | intron | ENST00000593531 | CTD-2207O23.3 | −1.17 | 1.7E−04 | 594 |
| X | 21816665 | 21817660 | intergenic | ENST00000465888 | MBTPS2 | −1.03 | 6.1E−04 | 995 |
| 9 | 131821742 | 131822331 | promoter | ENST00000474639 | FAM73B | 1.16 | 9.9E−04 | 589 |
| 18 | 60087362 | 60088390 | promoter | ENST00000591796 | RP11-640A1.4 | −1.02 | 1.3E−05 | 1028 |
| 2 | 187426114 | 187426881 | intergenic | ENST00000261023 | ITGAV | 1.22 | 1.6E−05 | 767 |
| 18 | 21269015 | 21270342 | promoter | ENST00000399516 | LAMA3 | −1.15 | 8.2E−10 | 1327 |
| 8 | 26165314 | 26165833 | intron | ENST00000523964 | PPP2R2A | −1.10 | 3.8E−04 | 519 |
| 6 | 43663358 | 43663937 | intergenic | ENST00000372133 | MRPS18A | −1.39 | 5.1E−06 | 579 |
| 10 | 8610021 | 8610921 | intergenic | ENST00000425516 | CHCHD3P1 | −1.01 | 7.6E−04 | 900 |
| 10 | 52753171 | 52754401 | intron | ENST00000373985 | PRKG1 | −1.13 | 1.3E−05 | 1230 |
| 17 | 69325178 | 69326441 | intergenic | ENST00000410631 | RNU6-305P | −1.35 | 2.1E−05 | 1263 |
| 12 | 22741552 | 22742171 | intergenic | ENST00000535801 | RP11-268P4.2 | −1.66 | 1.6E−05 | 619 |
| 4 | 77613059 | 77614188 | intron | ENST00000486758 | SHROOM3 | −1.47 | 8.2E−06 | 1129 |
| 22 | 42579385 | 42580044 | intron | ENST00000404876 | TCF20 | −1.28 | 4.7E−04 | 659 |
| 11 | 102800546 | 102801385 | intergenic | ENST00000260302 | MMP13 | −1.15 | 5.5E−05 | 839 |
| 1 | 168769107 | 168770153 | intergenic | ENST00000420691 | LINC00626 | −1.51 | 3.6E−04 | 1046 |
| 17 | 48968048 | 48968736 | intron | ENST00000514358 | TOB1-AS1 | −1.42 | 8.2E−09 | 688 |
| 6 | 131579205 | 131579893 | intron | ENST00000474850 | AKAP7 | −2.08 | 9.6E−06 | 688 |
| 5 | 111869063 | 111869538 | intergenic | ENST00000514243 | RP11-159K7.1 | −1.05 | 2.7E−05 | 475 |
| 10 | 9866325 | 9867152 | intergenic | ENST00000419836 | RP5-1051H14.2 | −1.51 | 4.2E−04 | 827 |
| 21 | 40174479 | 40175013 | intergenic | ENST00000360214 | ETS2 | −1.48 | 1.0E−04 | 534 |
| 3 | 169022989 | 169023782 | intron | ENST00000485957 | MECOM | −1.09 | 1.3E−04 | 793 |
| 10 | 74209572 | 74210383 | intron | ENST00000489666 | MICU1 | −1.48 | 9.2E−04 | 811 |
| 2 | 101441977 | 101442437 | intron | ENST00000430586 | AC092168.2 | −1.65 | 2.1E−05 | 460 |
| 15 | 71438884 | 71439471 | intron | ENST00000261862 | THSD4 | −1.30 | 6.6E−04 | 587 |
| 18 | 52434366 | 52434770 | intron | ENST00000586570 | RAB27B | −1.67 | 1.4E−06 | 404 |
| X | 17027964 | 17029048 | intron | ENST00000380064 | REPS2 | −1.40 | 4.7E−05 | 1084 |
| 4 | 74889262 | 74890088 | intergenic | ENST00000464637 | RN7SL218P | −1.21 | 4.3E−04 | 826 |
| 8 | 127836689 | 127837275 | intergenic | ENST00000519319 | PCAT1 | −1.41 | 3.6E−14 | 586 |
| 5 | 60757258 | 60757764 | intron | ENST00000252744 | ZSWIM6 | −1.20 | 6.5E−05 | 506 |
| 3 | 151576923 | 151578197 | intron | ENST00000475855 | RP11-454C18.2 | −1.62 | 1.9E−04 | 1274 |
| X | 17050088 | 17050991 | intron | ENST00000380064 | REPS2 | −1.22 | 4.1E−05 | 903 |
| 15 | 54081718 | 54082628 | intergenic | ENST00000383914 | RNU6-449P | −1.63 | 3.1E−04 | 910 |
| 4 | 115433283 | 115434630 | intergenic | ENST00000310836 | UGT8 | −1.23 | 6.4E−06 | 1347 |
| 6 | 131579943 | 131580553 | intron | ENST00000474850 | AKAP7 | −1.61 | 5.7E−07 | 610 |
| 7 | 65226259 | 65226827 | promoter | ENST00000384058 | SNORA15 | −1.65 | 3.5E−05 | 568 |
| 12 | 22715040 | 22716069 | intergenic | ENST00000542742 | RP11-359J14.3 | −1.65 | 1.9E−06 | 1029 |
| 8 | 8543551 | 8544101 | intergenic | ENST00000519106 | CLDN23 | −1.53 | 3.8E−05 | 550 |
| 17 | 56477290 | 56477780 | intron | ENST00000583841 | BZRAP1-AS1 | −1.09 | 9.2E−05 | 490 |
| 4 | 30903182 | 30904207 | intron | ENST00000511884 | PCDH7 | −1.09 | 1.8E−05 | 1025 |
| 12 | 13539722 | 13539939 | promoter | ENST00000532841 | C12orf36 | −1.34 | 4.5E−04 | 217 |
| 12 | 13539993 | 13540519 | promoter | ENST00000531049 | C12orf36 | −1.34 | 8.7E−05 | 526 |
| 4 | 109875916 | 109876470 | intron | ENST00000399126 | COL25A1 | −1.34 | 2.7E−04 | 554 |
| 15 | 97862475 | 97863361 | promoter | ENST00000559394 | RP11-315L6.1 | −1.52 | 4.7E−04 | 886 |
| 5 | 32302570 | 32303291 | intron | ENST00000513622 | MTMR12 | −1.36 | 4.6E−04 | 721 |
| 15 | 30110396 | 30110856 | intron | ENST00000473741 | TJP1 | −1.14 | 2.9E−04 | 460 |
| 4 | 175181121 | 175181620 | intron | ENST00000513696 | FBXO8 | −1.43 | 4.1E−05 | 499 |
| 21 | 16513635 | 16514425 | intergenic | ENST00000449746 | AF127577.12 | −1.38 | 2.9E−05 | 790 |
| 2 | 66800612 | 66801208 | promoter | ENST00000433396 | AC007392.3 | −1.41 | 5.1E−04 | 596 |
| 3 | 169097224 | 169097849 | intron | ENST00000485957 | MECOM | −1.23 | 1.3E−05 | 625 |
| 4 | 149352458 | 149353065 | intron | ENST00000511528 | NR3C2 | −1.02 | 9.1E−04 | 607 |
| 7 | 116452899 | 116453499 | promoter | ENST00000464223 | CAPZA2 | −1.44 | 9.7E−06 | 600 |
| 12 | 122595449 | 122596247 | intron | ENST00000319080 | MLXIP | −1.04 | 9.3E−04 | 798 |
| X | 110580244 | 110580776 | intron | ENST00000496551 | DCX | −1.33 | 6.6E−04 | 532 |
| 6 | 56558773 | 56559190 | promoter | ENST00000521104 | DST | −1.14 | 8.2E−04 | 417 |
| 8 | 71578881 | 71579614 | promoter | ENST00000276590 | LACTB2 | −1.09 | 2.1E−04 | 733 |
| 14 | 31503002 | 31503435 | intron | ENST00000555417 | AP4S1 | −1.35 | 3.1E−07 | 433 |
| 5 | 55053665 | 55054839 | intron | ENST00000504880 | SLC38A9 | −1.45 | 2.7E−05 | 1174 |
| 14 | 68631120 | 68631904 | intron | ENST00000557045 | RAD51B | −1.19 | 9.1E−04 | 784 |
| 4 | 53728083 | 53728700 | promoter | ENST00000515677 | RASL11B | −1.00 | 3.4E−05 | 617 |
| X | 17878644 | 17879810 | promoter | ENST00000545871 | RAI2 | −1.24 | 4.3E−04 | 1166 |
| 17 | 31121546 | 31122070 | intron | ENST00000583621 | MYO1D | −1.43 | 6.7E−04 | 524 |
| 13 | 73899238 | 73900358 | intergenic | ENST00000420129 | MARK2P12 | −1.07 | 2.2E−04 | 1120 |
| 7 | 117356474 | 117357412 | intron | ENST00000445366 | CTTNBP2 | −1.10 | 3.5E−04 | 938 |
| 17 | 71856589 | 71857110 | intergenic | ENST00000580370 | CTD-2532D12.5 | −1.05 | 9.1E−04 | 521 |
| 4 | 87863404 | 87863696 | intron | ENST00000511442 | AFF1 | −1.14 | 1.0E−04 | 292 |
| 14 | 90114844 | 90115344 | promoter | ENST00000516846 | Y_RNA | −1.33 | 6.3E−04 | 500 |
| 13 | 113339543 | 113340006 | intergenic | ENST00000356049 | C13orf35 | −1.43 | 4.0E−04 | 463 |
| 18 | 20714210 | 20714563 | promoter | ENST00000579963 | CABLES1 | −1.18 | 7.2E−05 | 353 |
| 13 | 106458613 | 106459355 | intergenic | ENST00000415294 | LINC00343 | −1.09 | 4.0E−04 | 742 |
| 18 | 10798713 | 10799240 | intron | ENST00000579112 | PIEZO2 | −1.31 | 2.7E−05 | 527 |
| 4 | 154110178 | 154111052 | intron | ENST00000437508 | TRIM2 | −1.00 | 4.2E−04 | 874 |
| 15 | 74305515 | 74306058 | intron | ENST00000564725 | PML | −1.73 | 6.2E−04 | 543 |
| 5 | 60550923 | 60551655 | intron | ENST00000503882 | CTC-436P18.3 | −1.45 | 5.6E−06 | 732 |
| 10 | 60228227 | 60229121 | intergenic | ENST00000373886 | BICC1 | −1.33 | 9.2E−05 | 894 |
| 2 | 151828282 | 151829233 | intergenic | ENST00000425983 | AC023469.2 | −1.10 | 9.5E−05 | 951 |
| 4 | 156625042 | 156625531 | intron | ENST00000513574 | GUCY1A3 | −1.16 | 1.8E−05 | 489 |
| 16 | 82061215 | 82061820 | intergenic | ENST00000563491 | HSD17B2 | −1.53 | 4.5E−06 | 605 |
| 3 | 27683392 | 27684170 | intergenic | ENST00000607601 | RP11-222K16.1 | −1.08 | 4.7E−04 | 778 |
| 8 | 38624299 | 38625022 | intron | ENST00000348567 | TACC1 | −1.03 | 8.6E−05 | 723 |
| 17 | 46018633 | 46019210 | promoter | ENST00000433001 | AC003665.1 | −1.15 | 2.2E−04 | 577 |
| 5 | 139544548 | 139545540 | exon | ENST00000607850_189600 | | −1.20 | 3.2E−04 | 992 |
| 4 | 30954382 | 30954826 | intron | ENST00000509759 | PCDH7 | −1.17 | 5.2E−04 | 444 |
| X | 35457520 | 35458562 | intergenic | ENST00000516602 | RNU6-1087P | −1.06 | 2.0E−04 | 1042 |
| 8 | 17652219 | 17652783 | intron | ENST00000381862 | MTUS1 | −1.53 | 5.6E−05 | 564 |
| 1 | 172137033 | 172137953 | intron | ENST00000523513 | DNM3 | −1.28 | 9.8E−04 | 920 |
| 4 | 155664739 | 155665500 | promoter | ENST00000510733 | LRAT | −1.59 | 1.9E−14 | 761 |
| 22 | 39317071 | 39317566 | promoter | ENST00000450216 | CTA-150C2.13 | −1.00 | 3.0E−04 | 495 |
| 11 | 22696063 | 22696714 | promoter | ENST00000433790 | GAS2 | −1.36 | 2.2E−06 | 651 |
| 5 | 66381100 | 66381787 | intron | ENST00000447738 | MAST4 | −1.09 | 1.3E−04 | 687 |
| 4 | 45648854 | 45650096 | intergenic | ENST00000363850 | RNU6-931P | −1.02 | 2.8E−04 | 1242 |
| 4 | 187564825 | 187565498 | intron | ENST00000441802 | FAT1 | −1.40 | 5.3E−07 | 673 |
| 15 | 53746791 | 53747925 | intergenic | ENST00000567224 | WDR72 | −1.40 | 5.0E−05 | 1134 |
| 4 | 105862880 | 105863326 | intron | ENST00000515649 | RP11-556I14.1 | −1.11 | 7.2E−04 | 446 |
| 4 | 77521435 | 77522140 | intron | ENST00000485780 | SHROOM3 | −1.13 | 7.2E−04 | 705 |
| 1 | 160512233 | 160512642 | exon | ENST00000534968_54273 | | −1.54 | 1.4E−04 | 409 |
| 4 | 25789258 | 25790342 | intron | ENST00000502949 | SEL1L3 | −1.49 | 6.0E−05 | 1084 |
| 21 | 15588231 | 15588966 | promoter | ENST00000400577 | RBM11 | −1.36 | 4.0E−05 | 735 |
| 15 | 23095116 | 23095978 | promoter | ENST00000559762 | RP11-566K19.5 | −1.14 | 6.8E−05 | 862 |
| 10 | 3598428 | 3598998 | intergenic | ENST00000426811 | RP11-482E14.2 | 1.87 | 5.3E−04 | 570 |
| 12 | 43309649 | 43310455 | intergenic | ENST00000603420 | RP11-510P12.1 | −1.26 | 3.0E−04 | 806 |
| 2 | 36008748 | 36009185 | intergenic | ENST00000431951 | MRPL50P1 | −1.81 | 2.2E−04 | 437 |
| 4 | 175547466 | 175548242 | intergenic | ENST00000274093 | GLRA3 | −1.43 | 1.7E−04 | 776 |
| 12 | 123129219 | 123129801 | intron | ENST00000356987 | HCAR1 | 1.17 | 3.5E−04 | 582 |
| 8 | 42082268 | 42083254 | promoter | ENST00000459183 | snoU13 | −1.26 | 5.4E−04 | 986 |
| 5 | 139598938 | 139599611 | intron | ENST00000509789 | CYSTM1 | −1.22 | 5.5E−04 | 673 |
| 7 | 121037949 | 121038214 | promoter | ENST00000411715 | CYCSP19 | −1.35 | 9.2E−07 | 265 |
| 4 | 94763615 | 94764289 | intergenic | ENST00000306011 | ATOH1 | −1.26 | 2.5E−04 | 674 |
| 12 | 12603953 | 12604650 | promoter | ENST00000605743 | RP11-253I19.4 | −1.48 | 8.2E−04 | 697 |
| 18 | 21075012 | 21075330 | intergenic | ENST00000269221 | C18orf8 | −1.19 | 3.7E−04 | 318 |
| X | 23925684 | 23926349 | promoter | ENST00000490078 | APOO | −1.02 | 8.0E−05 | 665 |
| 21 | 36250878 | 36251125 | intron | ENST00000486278 | RUNX1 | −1.32 | 7.2E−04 | 247 |
| 18 | 8329209 | 8329564 | intron | ENST00000577827 | PTPRM | −1.19 | 1.4E−04 | 355 |
| 2 | 73944031 | 73944360 | intergenic | ENST00000489476 | TPRKB | −1.29 | 2.2E−06 | 329 |
| 4 | 37491862 | 37492339 | intron | ENST00000508175 | C4orf19 | −1.21 | 5.4E−05 | 477 |
| Y | 2558421 | 2558773 | intergenic | ENST00000516032 | RNU6-1334P | −1.32 | 9.6E−05 | 352 |
| 15 | 63969949 | 63970349 | promoter | ENST00000559715 | HERC1 | −1.11 | 4.9E−04 | 400 |
| 18 | 19664513 | 19664896 | intergenic | ENST00000579830 | RP11-595B24.2 | −1.15 | 4.9E−07 | 383 |
| 4 | 74548559 | 74549428 | intergenic | ENST00000436089 | AC112518.3 | −1.19 | 4.1E−04 | 869 |
| 18 | 9422752 | 9423417 | intergenic | ENST00000262120 | TWSG1 | −1.51 | 3.5E−04 | 665 |
| 18 | 21464667 | 21465113 | promoter | ENST00000586751 | LAMA3 | −1.31 | 6.7E−05 | 446 |
| 4 | 48261077 | 48261668 | intron | ENST00000381501 | TEC | −1.28 | 8.2E−05 | 591 |
| 12 | 15305835 | 15306272 | promoter | ENST00000541243 | RERG-AS1 | −1.21 | 5.5E−05 | 437 |
| 4 | 105979088 | 105979826 | intron | ENST00000506386 | RP11-556I14.1 | −1.08 | 4.5E−04 | 738 |
| X | 2608934 | 2609490 | promoter | ENST00000381180 | CD99 | −1.44 | 9.0E−07 | 556 |
| 13 | 73544410 | 73545113 | promoter | ENST00000469712 | PIBF1 | −1.58 | 1.1E−06 | 703 |
| 4 | 55896756 | 55897737 | promoter | ENST00000517006 | RNU6-410P | −1.39 | 1.6E−04 | 981 |
| 4 | 13703459 | 13704075 | intron | ENST00000510907 | RP11-341G5.1 | −1.31 | 3.2E−05 | 616 |
| 14 | 64137369 | 64137812 | intergenic | ENST00000247225 | SGPP1 | −1.71 | 4.0E−04 | 443 |
| 12 | 26421726 | 26422408 | intron | ENST00000540392 | RP11-283G6.4 | −1.37 | 8.2E−05 | 682 |
| 18 | 4004111 | 4004976 | promoter | ENST00000582051 | DLGAP1 | −1.51 | 3.5E−04 | 865 |
| X | 16328282 | 16328968 | intergenic | ENST00000516839 | AC078993.1 | −1.31 | 2.9E−04 | 686 |
| X | 13012317 | 13012875 | intergenic | ENST00000451311 | TMSB4X | −1.75 | 2.4E−05 | 558 |
| 5 | 50521576 | 50522552 | intergenic | ENST00000468490 | CTD-2312P21.1 | −1.06 | 1.1E−04 | 976 |
| 4 | 87933836 | 87934323 | intron | ENST00000544085 | AFF1 | −1.28 | 1.1E−04 | 487 |
| 15 | 57619201 | 57619605 | promoter | ENST00000567319 | RP11-358M11.4 | −1.29 | 1.4E−04 | 404 |
| 8 | 118959719 | 118960347 | intron | ENST00000436216 | EXT1 | −1.20 | 9.7E−05 | 628 |
| 4 | 170106361 | 170107215 | intron | ENST00000508685 | SH3RF1 | −1.55 | 6.5E−06 | 854 |
| 14 | 23029755 | 23030313 | intergenic | ENST00000557595 | AE000662.92 | −1.03 | 2.5E−05 | 558 |
| 13 | 102392011 | 102392599 | intron | ENST00000376143 | FGF14 | −1.38 | 5.2E−04 | 588 |
| 4 | 186639663 | 186640609 | intron | ENST00000456060 | SORBS2 | −1.17 | 2.3E−04 | 946 |
| 17 | 35281035 | 35281678 | intron | ENST00000529264 | RP11-445F12.1 | −1.22 | 1.8E−05 | 643 |
| 18 | 19790101 | 19790813 | intergenic | ENST00000578741 | RP11-627G18.4 | −1.43 | 2.0E−07 | 712 |
| 4 | 85432843 | 85433341 | intergenic | ENST00000295886 | NKX6-1 | −1.15 | 5.1E−04 | 498 |
| 1 | 40357889 | 40358640 | intergenic | ENST00000397332 | MYCL | −1.30 | 7.4E−05 | 751 |
| 13 | 52532098 | 52532856 | intron | ENST00000542656 | ATP7B | −1.03 | 7.5E−05 | 758 |
| 12 | 92940036 | 92940836 | promoter | ENST00000459090 | snoU13 | −1.50 | 7.6E−05 | 800 |
| 4 | 158954507 | 158955331 | intergenic | ENST00000513850 | RP11-312A15.3 | −1.07 | 2.3E−06 | 824 |
| X | 132843583 | 132844339 | intron | ENST00000406757 | GPC3 | −1.88 | 1.5E−05 | 756 |
| 5 | 31048491 | 31049119 | intergenic | ENST00000495944 | RPL19P11 | −1.19 | 4.2E−04 | 628 |
| 18 | 24337137 | 24337871 | intron | ENST00000579964 | AQP4-AS1 | −1.32 | 5.9E−04 | 734 |
| 4 | 151435655 | 151436697 | intron | ENST00000513021 | LRBA | −1.10 | 6.8E−04 | 1042 |
| 4 | 72003550 | 72004695 | intergenic | ENST00000264485 | SLC4A4 | −1.27 | 2.5E−04 | 1145 |
| 16 | 52290147 | 52290849 | promoter | ENST00000408588 | AC007333.1 | −1.27 | 1.0E−04 | 702 |
| 18 | 19624260 | 19625733 | intron | ENST00000584898 | RP11-595B24.1 | −1.37 | 3.1E−07 | 1473 |
| 18 | 21209345 | 21209877 | promoter | ENST00000587763 | ANKRD29 | −1.41 | 3.0E−10 | 532 |
| 13 | 102399458 | 102399928 | intron | ENST00000376143 | FGF14 | −1.68 | 3.8E−06 | 470 |
| 4 | 106772105 | 106772882 | intron | ENST00000510876 | INTS12 | −1.11 | 2.2E−04 | 777 |
| 18 | 21290854 | 21291433 | promoter | ENST00000588044 | RPL23AP77 | −1.41 | 5.1E−05 | 579 |
| 13 | 108486621 | 108487030 | promoter | ENST00000449551 | FAM155A-IT1 | −1.35 | 5.9E−04 | 409 |
| 8 | 135029476 | 135029978 | intergenic | ENST00000605278 | RP11-157E21.2 | −1.27 | 5.8E−04 | 502 |
| 13 | 73614637 | 73615691 | intergenic | ENST00000437000 | PSMD10P3 | −1.09 | 3.1E−04 | 1054 |
| 18 | 60766821 | 60767604 | intergenic | ENST00000398117 | BCL2 | −1.01 | 1.8E−04 | 783 |
| 9 | 27385265 | 27386040 | intron | ENST00000603061 | MOB3B | −1.31 | 3.5E−04 | 775 |
| 17 | 72970801 | 72971274 | promoter | ENST00000532900 | HID1 | −1.29 | 2.8E−04 | 473 |
| X | 24163828 | 24164250 | intergenic | ENST00000427551 | ZFX-AS1 | −1.95 | 1.9E−06 | 422 |
| 18 | 70985941 | 70986635 | intergenic | ENST00000563172 | CTD-2354A18.1 | −1.61 | 3.1E−04 | 694 |
| 12 | 9880385 | 9880890 | intron | ENST00000327839 | CLECL1 | −1.33 | 8.1E−04 | 505 |
| 13 | 60181712 | 60182550 | intergenic | ENST00000400324 | DIAPH3 | −1.26 | 6.9E−05 | 838 |
| 15 | 90877324 | 90877942 | intergenic | ENST00000412799 | GABARAPL3 | −1.17 | 2.2E−04 | 618 |
| 18 | 59402679 | 59403762 | intron | ENST00000590968 | RP11-879F14.1 | −1.28 | 8.2E−04 | 1083 |
| 14 | 39308853 | 39309445 | promoter | ENST00000557440 | LINC00639 | −1.08 | 1.4E−04 | 592 |
| 4 | 22943322 | 22944138 | intergenic | ENST00000511453 | RP11-412P11.1 | −1.16 | 7.5E−04 | 816 |
| 4 | 139833077 | 139833445 | intron | ENST00000507038 | RP11-371F15.3 | −1.03 | 2.4E−04 | 368 |
| 18 | 19686422 | 19686904 | intergenic | ENST00000579830 | RP11-595B24.2 | −1.11 | 6.5E−06 | 482 |
| 10 | 43137085 | 43137382 | intergenic | ENST00000486614 | ZNF33B | −1.35 | 2.5E−04 | 297 |
| 20 | 15119226 | 15119713 | intron | ENST00000310348 | MACROD2 | −1.51 | 5.0E−07 | 487 |
| 21 | 36168889 | 36169428 | intron | ENST00000399240 | RUNX1 | −1.09 | 9.0E−04 | 539 |
| 18 | 4017582 | 4018096 | intron | ENST00000577430 | DLGAP1 | −1.26 | 7.7E−04 | 514 |
| 5 | 132208952 | 132209463 | promoter | ENST00000485457 | LEAP2 | −1.25 | 7.5E−04 | 511 |
| 7 | 115979679 | 115980039 | intron | ENST00000446355 | AC002066.1 | −1.22 | 4.9E−04 | 360 |
| 18 | 55102256 | 55103165 | promoter | ENST00000581316 | AC090340.1 | −1.02 | 3.1E−04 | 909 |
| 4 | 170035695 | 170036113 | intron | ENST00000284637 | SH3RF1 | −1.61 | 1.7E−04 | 418 |
| X | 15872339 | 15873736 | promoter | ENST00000421527 | AP1S2 | −1.09 | 2.1E−08 | 1397 |
| 4 | 177114274 | 177114599 | promoter | ENST00000515234 | SPATA4 | −1.69 | 2.3E−04 | 325 |
| 18 | 40105871 | 40106286 | intron | ENST00000589068 | LINC00907 | −1.40 | 7.3E−04 | 415 |
| 13 | 99300363 | 99300982 | intergenic | ENST00000430810 | CALM2P4 | −1.25 | 1.6E−04 | 619 |
| 7 | 12969053 | 12969525 | intergenic | ENST00000441256 | RBMX2P4 | −1.31 | 3.5E−04 | 472 |
| X | 117907769 | 117908146 | intron | ENST00000371637 | IL13RA1 | −1.28 | 2.2E−04 | 377 |
| 1 | 12050437 | 12051116 | intron | ENST00000412236 | MFN2 | 1.27 | 8.3E−04 | 679 |
| 4 | 171147427 | 171147816 | intergenic | ENST00000504509 | RP11-789C1.1 | −1.58 | 8.7E−04 | 389 |
| 12 | 13158692 | 13159059 | intron | ENST00000543321 | RP11-377D9.3 | −1.66 | 2.7E−04 | 367 |
| 8 | 29595979 | 29596739 | intron | ENST00000506121 | LINC00589 | −1.10 | 3.4E−05 | 760 |
| 8 | 22312699 | 22313062 | intron | ENST00000522000 | PPP3CC | −1.41 | 2.6E−04 | 363 |
| 4 | 103811017 | 103811934 | intron | ENST00000514972 | SLC9B1 | −1.11 | 5.3E−04 | 917 |
| 8 | 8549498 | 8549897 | intergenic | ENST00000519106 | CLDN23 | −1.38 | 3.2E−04 | 399 |
| 4 | 106818891 | 106819676 | promoter | ENST00000513430 | NPNT | −1.04 | 7.7E−04 | 785 |
| 10 | 6343519 | 6344014 | intron | ENST00000399868 | RP11-563J2.2 | −1.02 | 2.6E−04 | 495 |
| 9 | 78528856 | 78529314 | promoter | ENST00000459505 | AL359253.1 | −1.59 | 9.4E−06 | 458 |
| 5 | 17114415 | 17114792 | intron | ENST00000606445 | BASP1 | −1.06 | 3.2E−04 | 377 |
| X | 15624226 | 15624853 | intron | ENST00000421585 | GS1-594A7.3 | −1.44 | 5.7E−04 | 627 |
| 18 | 21189439 | 21189988 | intron | ENST00000591617 | ANKRD29 | −1.54 | 1.6E−05 | 549 |
| 10 | 115312349 | 115312929 | promoter | ENST00000541666 | HABP2 | −1.19 | 9.3E−04 | 580 |
| 6 | 119915982 | 119916519 | intergenic | ENST00000368468 | MAN1A1 | −1.26 | 8.3E−04 | 537 |
| 19 | 39646961 | 39647663 | promoter | ENST00000599657 | PAK4 | 1.28 | 2.7E−04 | 702 |
| 4 | 157873335 | 157873855 | intron | ENST00000422544 | PDGFC | −1.58 | 3.2E−04 | 520 |
| 4 | 77510524 | 77510923 | intron | ENST00000485780 | SHROOM3 | −1.06 | 2.6E−04 | 399 |
| 3 | 7246159 | 7246840 | intron | ENST00000435689 | GRM7 | −1.25 | 7.7E−04 | 681 |
| 18 | 9673064 | 9673873 | intergenic | ENST00000581937 | KRT18P8 | −1.16 | 5.7E−04 | 809 |
| 18 | 71068317 | 71068856 | intergenic | ENST00000563172 | CTD-2354A18.1 | −1.22 | 8.9E−04 | 539 |
| 4 | 18814749 | 18815500 | intergenic | ENST00000503815 | RP11-608B3.1 | −1.54 | 6.5E−04 | 751 |
| 18 | 9736984 | 9737287 | promoter | ENST00000578806 | RP11-692N5.2 | −1.58 | 7.5E−04 | 303 |
| 12 | 21597079 | 21597766 | intron | ENST00000538582 | PYROXD1 | −1.38 | 7.6E−05 | 687 |
| 18 | 28981489 | 28981834 | promoter | ENST00000581452 | RP11-534N16.1 | −1.56 | 5.4E−04 | 345 |
| 5 | 37165920 | 37166523 | intron | ENST00000511824 | C5orf42 | −1.15 | 1.4E−04 | 603 |
| 12 | 60566172 | 60566790 | intergenic | ENST00000551882 | RP11-335M9.1 | −1.26 | 6.7E−04 | 618 |
| 18 | 26372413 | 26372889 | intergenic | ENST00000582726 | RP11-510D21.1 | −1.30 | 1.3E−04 | 476 |
| X | 3631095 | 3632157 | promoter | ENST00000262848 | PRKX | −1.25 | 2.1E−06 | 1062 |
| 18 | 3250757 | 3251051 | promoter | ENST00000578562 | MYL12A | −1.05 | 8.9E−04 | 294 |
| 18 | 65288323 | 65288979 | intron | ENST00000583687 | RP11-638L3.1 | −1.51 | 4.2E−04 | 656 |
| 5 | 144843814 | 144844163 | intergenic | ENST00000510259 | PRELID2 | −1.41 | 1.0E−04 | 349 |
| 18 | 21544367 | 21545241 | intron | ENST00000582300 | RP11-403A21.1 | −1.06 | 4.2E−04 | 874 |
| 12 | 71557965 | 71558303 | intron | ENST00000549421 | TSPAN8 | −1.61 | 1.8E−04 | 338 |
| 12 | 13025022 | 13026103 | intergenic | ENST00000459725 | RPL13AP20 | −1.09 | 2.5E−04 | 1081 |
| 12 | 71555389 | 71555659 | intron | ENST00000549421 | TSPAN8 | −1.60 | 5.5E−04 | 270 |
| 5 | 54660393 | 54660916 | intron | ENST00000545714 | SKIV2L2 | −1.16 | 7.1E−04 | 523 |
| 6 | 106894847 | 106895225 | intergenic | ENST00000365516 | RNA5SP211 | −1.80 | 2.0E−04 | 378 |
| X | 77192772 | 77193146 | intron | ENST00000602791 | RP5-1000K24.2 | −1.23 | 2.4E−04 | 374 |
| 18 | 12000289 | 12000722 | promoter | ENST00000588863 | IMPA2 | −2.16 | 3.4E−05 | 433 |
| 18 | 3456781 | 3457062 | promoter | ENST00000472042 | TGIF1 | −1.75 | 3.1E−05 | 281 |
| 5 | 43893907 | 43894383 | intergenic | ENST00000508829 | RP11-8L21.1 | −1.61 | 1.2E−04 | 476 |
| 13 | 35515748 | 35516975 | promoter | ENST00000379939 | NBEA | −1.58 | 3.8E−11 | 1227 |
| 18 | 26374435 | 26374857 | intergenic | ENST00000582726 | RP11-510D21.1 | −1.31 | 2.1E−04 | 422 |
| 15 | 89668375 | 89668644 | intron | ENST00000562073 | ABHD2 | −1.65 | 2.6E−04 | 269 |
| 4 | 37978642 | 37979668 | promoter | ENST00000446803 | TBC1D1 | −1.06 | 6.4E−05 | 1026 |
| 13 | 77498752 | 77499091 | intergenic | ENST00000426582 | BTF3P11 | −1.37 | 8.4E−04 | 339 |
| X | 105961933 | 105962318 | intron | ENST00000324342 | RNF128 | −1.51 | 9.7E−04 | 385 |
| 14 | 56355837 | 56356276 | intergenic | ENST00000569625 | RP11-1012E15.1 | −1.08 | 3.5E−04 | 439 |
| 3 | 66543117 | 66543471 | intron | ENST00000475366 | LRIG1 | −1.23 | 1.5E−04 | 354 |
| 4 | 4501198 | 4501552 | intron | ENST00000512780 | STX18 | −1.44 | 8.4E−04 | 354 |
| 15 | 90401815 | 90402255 | intron | ENST00000559629 | C15orf38-AP3S2 | −1.93 | 2.5E−05 | 440 |
| 7 | 13005419 | 13005842 | intergenic | ENST00000441256 | RBMX2P4 | −1.15 | 2.6E−04 | 423 |
| 14 | 37798337 | 37798669 | intron | ENST00000556940 | MIPOL1 | −1.52 | 6.7E−06 | 332 |
| 17 | 48774453 | 48774711 | promoter | ENST00000364470 | Y_RNA | −1.16 | 6.7E−04 | 258 |
| 13 | 32519681 | 32520190 | intron | ENST00000428783 | EEF1DP3 | −1.12 | 7.1E−06 | 509 |
| 17 | 10640501 | 10640980 | intron | ENST00000583012 | CTC-297N7.5 | −1.12 | 5.2E−05 | 479 |
| 8 | 142140988 | 142141629 | promoter | ENST00000517908 | RP11-809O17.1 | 1.23 | 3.7E−04 | 641 |
| 9 | 45008582 | 45009082 | intron | ENST00000421848 | RP11-374M1.4 | −1.17 | 6.0E−04 | 500 |
| 18 | 19748853 | 19749787 | promoter | ENST00000583490 | GATA6-AS1 | −1.54 | 1.0E−06 | 934 |
| 8 | 17646298 | 17647375 | intron | ENST00000381862 | MTUS1 | −1.20 | 5.7E−04 | 1077 |
| 17 | 618801 | 619322 | promoter | ENST00000437048 | VPS53 | −1.27 | 6.7E−04 | 521 |
| 13 | 93125967 | 93126657 | intron | ENST00000377067 | GPC5 | −1.64 | 6.0E−04 | 690 |
| 10 | 65479061 | 65479739 | intron | ENST00000444770 | RP11-170M17.1 | −1.72 | 1.2E−06 | 678 |
| 3 | 19189370 | 19190217 | promoter | ENST00000452398 | KCNH8 | −1.61 | 1.6E−05 | 847 |
| 1 | 59245356 | 59246066 | intergenic | ENST00000371222 | JUN | −1.23 | 4.7E−05 | 710 |
| 9 | 105629671 | 105630230 | intergenic | ENST00000430854 | RP11-338N12.1 | −1.50 | 3.7E−04 | 559 |
| 2 | 134946547 | 134947309 | intron | ENST00000409645 | MGAT5 | −1.11 | 8.1E−04 | 762 |
| 12 | 113905094 | 113906232 | intron | ENST00000261731 | LHX5 | 1.03 | 7.6E−04 | 1138 |
| 13 | 78271260 | 78272125 | promoter | ENST00000466548 | SLAIN1 | −1.58 | 4.6E−08 | 865 |
| 14 | 68658282 | 68659082 | intron | ENST00000557045 | RAD51B | −1.08 | 4.4E−05 | 800 |
| X | 22003441 | 22003730 | intron | ENST00000415881 | SMS | −1.08 | 1.9E−04 | 289 |
| 7 | 38903200 | 38903772 | intron | ENST00000457055 | VPS41 | −1.16 | 4.2E−06 | 572 |
| 17 | 53510366 | 53511001 | intergenic | ENST00000262065 | MMD | −1.11 | 1.7E−04 | 635 |
| 3 | 194353440 | 194353664 | promoter | ENST00000447139 | AC046143.3 | 1.07 | 8.2E−04 | 224 |
| 12 | 15373831 | 15374573 | promoter | ENST00000537717 | RERG | −1.02 | 2.1E−05 | 742 |
| 15 | 52199610 | 52200183 | promoter | ENST00000606352 | U6 | −1.12 | 2.3E−08 | 573 |
| Y | 2476943 | 2477666 | intergenic | ENST00000516032 | RNU6-1334P | −1.19 | 1.4E−04 | 723 |
| 17 | 46024345 | 46024764 | promoter | ENST00000580372 | RP11-6N17.6 | −1.16 | 9.1E−04 | 419 |
| X | 1710260 | 1710695 | promoter | ENST00000381261 | AKAP17A | −1.01 | 6.7E−04 | 435 |
| Y | 2558832 | 2559585 | intergenic | ENST00000516032 | RNU6-1334P | −1.33 | 8.4E−07 | 753 |
| 18 | 23806089 | 23807166 | promoter | ENST00000418698 | TAF4B | −1.39 | 2.7E−05 | 1077 |
| 4 | 69598563 | 69599228 | intergenic | ENST00000509261 | RP11-1267H10.4 | −1.31 | 9.2E−08 | 665 |
| 12 | 6419391 | 6420221 | promoter | ENST00000396988 | PLEKHG6 | −1.05 | 4.5E−04 | 830 |
| 18 | 2984812 | 2985290 | promoter | ENST00000584915 | LPIN2 | −1.58 | 6.0E−05 | 478 |
| X | 20392961 | 20393546 | intergenic | ENST00000517169 | RN7SKP183 | −1.03 | 8.9E−04 | 585 |
| 15 | 66124847 | 66125582 | intron | ENST00000568850 | RAB11A | −1.07 | 6.5E−06 | 735 |
| 4 | 119273882 | 119274465 | promoter | ENST00000296498 | PRSS12 | −1.00 | 5.3E−05 | 583 |
| 3 | 19188141 | 19189179 | promoter | ENST00000328405 | KCNH8 | −1.13 | 2.3E−04 | 1038 |
| 11 | 94335540 | 94336653 | intron | ENST00000537874 | RP11-867G2.8 | −1.14 | 1.1E−05 | 1113 |
| 18 | 29665492 | 29665879 | intron | ENST00000583184 | RP11-5316.2 | −1.70 | 2.9E−04 | 387 |
| 5 | 176513355 | 176514471 | promoter | ENST00000513166 | FGFR4 | −1.02 | 2.6E−04 | 1116 |
| 18 | 12376764 | 12377928 | promoter | ENST00000590811 | AFG3L2 | −1.05 | 4.2E−04 | 1164 |
| X | 33780627 | 33780788 | intron | ENST00000445233 | RP11-305F18.1 | −1.32 | 9.7E−04 | 161 |
| X | 83441953 | 83443818 | promoter | ENST00000460730 | RPS6KA6 | −1.04 | 1.3E−04 | 1865 |
| 4 | 15679072 | 15679693 | intron | ENST00000514541 | FBXL5 | −1.30 | 6.6E−10 | 621 |
| 3 | 194432537 | 194433012 | intron | ENST00000423318 | AC090505.6 | 1.01 | 6.1E−04 | 475 |
| 17 | 36070163 | 36070788 | intron | ENST00000560016 | HNF1B | −1.79 | 6.3E−11 | 625 |
| 18 | 28551397 | 28551656 | intron | ENST00000583580 | RP11-25I11.1 | −1.93 | 8.6E−05 | 259 |
| 18 | 21795580 | 21796435 | promoter | ENST00000384039 | RNU6-435P | −1.00 | 9.5E−04 | 855 |
| 4 | 89897580 | 89898181 | intron | ENST00000509094 | FAM13A | −1.15 | 9.6E−04 | 601 |
| 8 | 40013191 | 40014286 | intergenic | ENST00000315792 | C8orf4 | −1.03 | 3.5E−04 | 1095 |
| 4 | 52883991 | 52884363 | promoter | ENST00000343457 | LRRC66 | −1.30 | 2.6E−04 | 372 |
| 3 | 66692481 | 66692862 | intergenic | ENST00000459863 | RPL21P41 | −1.43 | 8.0E−04 | 381 |
| 18 | 19748357 | 19748632 | promoter | ENST00000579431 | GATA6-AS1 | −1.26 | 1.8E−04 | 275 |
| 5 | 58145773 | 58146112 | intron | ENST00000510198 | CTD-2176121.2 | −1.35 | 1.2E−05 | 339 |
| 5 | 40485204 | 40485821 | intergenic | ENST00000583717 | AC108105.1 | −1.22 | 2.4E−04 | 617 |
| 7 | 64532350 | 64532740 | promoter | ENST00000384334 | SNORA15 | −1.70 | 6.4E−06 | 390 |
| 18 | 21450963 | 21451245 | promoter | ENST00000269217 | LAMA3 | −1.41 | 1.8E−07 | 282 |
| 15 | 57899754 | 57900281 | intron | ENST00000569089 | MYZAP | −1.17 | 1.9E−06 | 527 |
| 12 | 27425172 | 27426386 | intron | ENST00000543246 | STK38L | −1.11 | 2.0E−05 | 1214 |
| 2 | 306486 | 306655 | promoter | ENST00000592090 | AC079779.5 | 1.89 | 9.5E−05 | 169 |
| 16 | 83983871 | 83984533 | promoter | ENST00000361711 | OSGIN1 | 1.35 | 7.1E−05 | 662 |
| 7 | 591580 | 592225 | promoter | ENST00000517177 | AC147651.2 | 1.23 | 1.8E−04 | 645 |

| | |
|---|---|
| Median | 595.5 |
| Min. | 151 |
| Max. | 2612 |

### 8.2 Example 2: qPCR Array Methodology

### 8.2.1 ATAC-qPCRarray Preparation:

ATAC-qPCRarray platform technology was developed in order to cross-validate the chromatin accessibility signature (as obtained by ATAC-seq as described in Example 1 above) classifying PDAC patients into recurrent and non-recurrent groups.

An array was prepared on a desired format. The array was prepared by taking the coordinates of previously identified open chromatin peaks, the start and end loci.

Oligonucleotide primers were designed which are complementary to these open chromatin regions (between the start and the end loci) to amplify the specific targeted region of the chromatin accessible regions. A set of oligonucleotide primers targeting a specific open chromatin region was placed in a PCR amplification plate on an array format (FIGS. 4A-4B) as described in Example 2. One or more sets of oligonucleotide primers may target a specific open chromatin region.

An exemplary PDAC array may target fewer than 100 chromatin regions identified in Table 1 and/or Table 2.

TABLE 2

| Chromosome | Start | End | Annotation | Symbol |
|---|---|---|---|---|
| chr12 | 19,426,717 | 19,427,323 | Intron | PLEKHA5 |
| chr3 | 105,525,021 | 105,525,647 | Intron | CBLB |
| chr2 | 145,077,946 | 145,078,518 | Intron | GTDC1 |
| chr4 | 40,674,840 | 40,675,527 | Intergenic | RBM47 |
| chr5 | 110,062,076 | 110,062,896 | Promoter | TMEM232 |
| chr9 | 75,766,438 | 75,767,040 | Promoter | ANXA1 |
| chr4 | 81,094,544 | 81,094,954 | Intergenic | PRDM8 |
| chr13 | 74,289,467 | 74,290,340 | Intron | KLF12 |
| chr7 | 35,762,586 | 35,763,101 | Intergenic | LOC100506725 |
| chr7 | 7,984,167 | 7,984,977 | Intergenic | GLCCI1 |
| chr12 | 76,371,552 | 76,372,217 | Intergenic | PHLDA1 |
| chr4 | 101,960,452 | 101,960,971 | Intron | PPP3CA |
| chr2 | 145,140,347 | 145,140,933 | Intergenic | GTDC1 |
| chr6 | 135,889,386 | 135,889,933 | Intron | LINC00271 |
| chr3 | 109,523,484 | 109,524,020 | Intergenic | MIR4445 |
| chr3 | 105,465,536 | 105,466,279 | Intron | CBLB |
| chr4 | 36,256,926 | 36,257,681 | Promoter | LOC439933 |
| chr3 | 189,042,301 | 189,043,047 | TTS | TPRG1 |
| chr11 | 85,534,713 | 85,535,433 | Intergenic | SYTL2 |
| chr13 | 99,871,034 | 99,871,786 | Intron | UBAC2 |
| chr7 | 143,072,243 | 143,073,098 | Intergenic | FAM131B |
| chr5 | 58,419,377 | 58,419,920 | Intron | PDE4D |
| chr6 | 128,292,926 | 128,293,807 | Intron | PTPRK |
| chr2 | 161,250,545 | 161,251,085 | Intron | RBMS1 |
| chr13 | 74,600,816 | 74,601,467 | Intron | KLF12 |
| chr2 | 137,084,604 | 137,085,454 | Intergenic | CXCR4 |
| chr3 | 71,542,455 | 71,543,074 | Intron | FOXP1 |
| chr1 | 192,485,704 | 192,486,501 | Intergenic | RGS1 |
| chr14 | 20,801,062 | 20,801,767 | Promoter | CCNB1IP1 |
| chr4 | 102,448,189 | 102,448,931 | Intergenic | FLJ20021 |
| chr10 | 6,540,520 | 6,541,245 | Intron | PRKCQ |
| chr1 | 183,602,686 | 183,603,277 | Intron | ARPC5 |
| chr18 | 60,087,645 | 60,088,463 | Intergenic | TNFRSF11A |
| chr14 | 70,082,213 | 70,082,726 | Intron | SUSD6 |
| chr5 | 50,006,858 | 50,007,607 | Intron | PARP8 |
| chr4 | 114,468,552 | 114,469,125 | Exon | CAMK2D |
| chr6 | 137,710,174 | 137,710,795 | Intergenic | OLIG3 |
| chr5 | 67,575,735 | 67,576,365 | Intron | PIK3R1 |
| chr1 | 66,815,639 | 66,816,413 | Intron | PDE4B |
| chr1 | 84,944,781 | 84,945,643 | Intron | RPF1 |
| chr8 | 134,315,289 | 134,316,163 | Intergenic | NDRG1 |
| chr6 | 143,223,647 | 143,224,367 | Intron | HIVEP2 |
| chr7 | 130,920,376 | 130,920,953 | Intron | MKLN1 |
| chr6 | 130,004,960 | 130,005,621 | Intron | ARHGAP18 |
| chr10 | 81,082,715 | 81,083,458 | Intergenic | PPIF |
| chr4 | 122,859,782 | 122,860,343 | Intron | TRPC3 |
| chr12 | 90,343,531 | 90,344,571 | TTS | LINC02399 |
| chr2 | 161,284,266 | 161,284,976 | Intron | RBMS1 |
| chr6 | 36,409,032 | 36,409,598 | Intron | PXT1 |
| chr13 | 41,593,129 | 41,593,701 | Promoter | ELF1 |
| chr6 | 138,132,378 | 138,132,988 | Intergenic | TNFAIP3 |
| chr8 | 116,660,226 | 116,660,896 | Intron | TRPS1 |
| chr8 | 91,639,845 | 91,640,621 | Intron | TMEM64 |
| chr3 | 95,424,587 | 95,425,386 | Intergenic | MTHFD2P1 |
| chr12 | 92,754,202 | 92,754,753 | Intergenic | CLLU1 |
| chr7 | 92,250,105 | 92,250,723 | Intron | CDK6 |
| chr3 | 111,386,503 | 111,387,218 | Intergenic | PLCXD2 |
| chr5 | 25,569,096 | 25,569,799 | Intergenic | LINC02211 |
| chr3 | 150,102,582 | 150,103,090 | Intergenic | TSC22D2 |
| chr12 | 75,904,898 | 75,905,901 | Promoter | KRR1 |
| chr3 | 112,709,379 | 112,710,528 | Exon | GTPBP8 |
| chr11 | 14,436,552 | 14,437,156 | Intergenic | RRAS2 |
| chr6 | 106,642,940 | 106,643,882 | Intron | ATG5 |
| chr3 | 183,003,467 | 183,004,223 | Intron | MCF2L2 |
| chr4 | 104,020,729 | 104,021,363 | Promoter | BDH2 |
| chr2 | 162,164,500 | 162,165,398 | 5′ UTR | PSMD14 |
| chr6 | 131,520,609 | 131,521,624 | Intron | AKAP7 |

### 8.2.2. Library Preparation:

ATAC libraries were prepared as described in detail below. Briefly, intact nuclei were extracted from a biological sample. A Tn5 transposase complex was added to the intact nuclei. Following an incubation, transposed DNA fragments were extracted from the reaction solution and amplified to provide ATAC libraries.

The preparation of tumor specimens followed the procedure outlined below: first EpCAM+PDAC malignant cells, or CD8+T-lymphocytes were isolated from the tumor and peripheral blood respectively, and then ATAC-libraries were made (the details of the methodology in given below).

8.2.2.1 Making Single-Cell Suspension from PDAC FNA/Laparoscopic Surgical/Surgically Resected Specimens:

The FNA/laparoscopic surgical/surgically resected specimens were taken into a 50-ml Gentle-MACS “C” tube containing the digestion buffer: 5 ml of media (MEM+ protease inhibitor)+100 μl of liberase (Roche)+50 μl P188 (15 mM stock)+5 μl DNAse-1 (10 mg/ml stock)+37.5 μl CaCl₂) (1M stock) and the tube was placed in Gentle-MACS tissue dissociator machine for 60 min at 37° C. After incubation, 5 ml of MACS buffer was added, and the suspension filtered through 40 μM filter (BD cell strainer) into another 50 ml microfuge tube. The tube was centrifuged @500×g for 5 min at 4° C. and the supernatant discarded. 500 μL of ACK lysing buffer was added to the pellet, incubated for 5 min at RT then diluted immediately with 4.5 ml of MACS buffer (BSA diluted 1:20 with Auto-MACS rinsing solution). The tube was centrifuged @500×g for 5 min at 4° C. and the supernatant discarded. The cell pellet was re-suspended in 50 μL of MACS buffer and 50 μL of FcR Blocking Reagent and 50 μL of CD326 (EpCAM) Micro-Beads were added. The mixture was mixed well and refrigerated for 30 minutes (4-8° C.) but not on ice. After the incubation, the cells were washed once by adding 5 ml of MACS buffer and centrifuged at 500×g for 5 minutes at 4° C. The supernatant was aspirated completely. The pellet was re-suspended in 500 μL of MACS buffer and proceed to magnetic separation.

8.2.3. Magnetic Separation of EpCAM+ Cells with LS Columns

A 15 ml tube was used for collection of the effluents (start preparing the column by rinsing with 3 ml MACS buffer while centrifuging the cell suspension). The cell suspension 500 μL was applied onto the column. “Unlabeled” cells (anything other than epithelial cells) that pass through were collected and the column was washed with 3×3 ml of buffer as effluent. Washing steps were performed by adding buffer three times. The column was removed from the separator and placed on a 15 ml collection tube. 5 ml of buffer was pipetted onto the column. The magnetically labeled cells were flushed out by firmly pushing the plunger into the column. (To increase the purity of the magnetically labeled fraction, the cells may be passed over a new, freshly prepared column.) The cells (˜5 ml total suspension) were pelleted down @500×g for 5 min at 4° C. The unlabeled cells (˜12.5 ml total suspension from previous step) were also pelleted down @500×g for 5 min at 4° C. Supernatant was discarded and labeled cells were re-suspended in 200 μL of 1× cold PBS. The cells were counted, and only epithelial cells fraction were used for ATAC-library preparation utilizing 10,000-50,000 cells, and the remaining cells were stored for DNA/RNA extraction (later with Qiagen All-prep DNA-RNA kit). The “Effluent” fraction was pelleted down and stored at −80° C. along with the epithelial cell fraction for future DNA/RNA extraction in order to utilize it as control for checking epithelial enrichment.

8.2.4. Continue with Transposition Reaction on the Isolated Cells

10,000-50,000 cells were taken in each of the two 1.5 ml microfuge tubes (in duplicates) and centrifuged for 5 min at 500×g at 4° C. Supernatant was discarded and the cell pellet was re-suspended by pipetting up and down in 50 μl of cold lysis buffer. The re-suspended pellet was centrifuged immediately for 10 min at 500×g at 4° C. This step affords lysis of cells with nonionic detergent and generated a crude nuclei preparation. The supernatant was discarded, and the crude nuclei preparation was used in the transposition reaction.

### 8.2.5. Transposition Reaction and Purification

Transposition reaction and purification were performed as described in Buenrostro, Nat Methods (2013) with modifications. The cell pellet was placed on ice. Transposition reaction mixture:
a. In 100-μL for a duplicate library reaction:
   i. 50-μL TN5 buffer TD (2×reaction buffer from Nextera kit)
   ii. 45-μL nuclease-free water
   iii. 5-μL TN5 enzyme TDE1 (Nextera Tn5 Transposase from Nextera kit)
b. The transposition reaction mixture was incubated at 37° C. for 30 min with gentle mixing to increase fragment yield.

The transposition reaction mixture was incubated at 37° C. for 30 min with gentle mixing to increase fragment yield. Purification was performed using The transposition reaction mixture was eluted in 20-μL elution buffer (Qiagen MinElute PCR Purification Kit) before PCR. Purified DNA was stored at −20° C. if necessary.

### 7.3.6. PCR Amplification of Transposed DNA Fragments

10-μL elute was taken into the 50-μL PCR-reaction and then the usual protocol was followed with the primer pairs as described in Buenrostro, Nat Methods (2013) (supplement). The amplicons were purified with Qiagen mini-elute PCR cleanup kit.

The following was combined in a 0.2 ml PCR tube:
10 μl transposed DNA (or the cleaned product of the first PCR)
10 μl nuclease-free H₂O
2.5 μl 25 μM PCR Primer 1.
2.5 μl 25 μM Barcoded PCR Primer 2 (1 through 24-all primers, forward (primer 1) and reverse (primer 2) from Buenrostro, Nat Methods (2013) (supplement)
25 μl NEBNext High-Fidelity 2×PCR Master Mix

Primers and PCR conditions were optimized for amplifying large-molecular-weight fragments from low-input material. Integrated DNA Technologies (IDT) synthesized all primers—with no additional modifications. Samples were barcoded appropriately for subsequent pooling and sequencing.

Thermal cycle conditions were as follows:

| | | |
|---|---|---|
| 1 cycle: | 5 min | 72° C. |
| | 30 sec | 98° C. |
| 12 cycles: | 10 sec | 98° C. |
| | 30 sec | 63° C. |
| | 1 min | 72° C. |

The first 5-min extension at 72° C. allowed for extension of both ends of the primer after transposition, thereby generating amplifiable fragments.

Amplified library was purified using Qiagen MinElute PCR Purification Kit. The purified library was eluted in 20 μl elution buffer (Buffer EB from the MinElute kit consisting of 10 mM Tris·Cl, pH 8). The column was dried prior to adding elution buffer to avoid ethanol contamination in the final library. Typically, the nanodrop concentration after 12 cycle PCR is ˜ 10 fold more than the before PCR (The concentration of DNA eluted from the column ought to be approximately 30 nM; however, 5-fold variation is possible and not detrimental). The quality of purified libraries was assessed using a Bioanalyzer High-Sensitivity DNA Analysis kit (Agilent).

7.3.7. qPCR of the Libraries with the qPCRarray

The ATAC-qPCR-array will be made by selected differentially represented regions and that will be normalized with control regions. Typically, control regions are ACRs that exhibit no significant or detectable differential expression between the two phenotypes of comparison. No Template Control (n) will be used as negative control. The specific set of oligonucleotide primers will be designed using publicly available Primer 3 software. The primers will be optimized and the sequence of each amplicon will be confirmed by Sanger sequencing. The set of optimized primers will be used to make the ATAC-qPCRarrays using 96-well or 384-well format (as displayed in FIGS. 4A-4B). The set of test regions will be selected based on the differentially represented ACRs between two phenotypic groups, e.g., poor versus good prognosis, or responder versus non-responder groups of patients to a certain therapy (e.g., chemotherapy, targeted therapy, or immunotherapy). Quantitative measurement of target accessible region relative to control regions will be performed in triplicates using Power SYBR Green PCR Master Mix (Applied Biosystems, Warrington, UK cat #4368706) following the manufacturer's recommendations. Relative quantitation of the chromatin accessibility will be defined by 2^{-ΔCt}, where ΔCt=Ct_{target} region ⁻Ct_{control region}. A score will be assigned to each patient based on the relative quantitation of the ACRs by the ATAC-qPCR array.

### 8.3 Example 3—ATAC-qPCRarray for Evaluating PDAC Recurrence

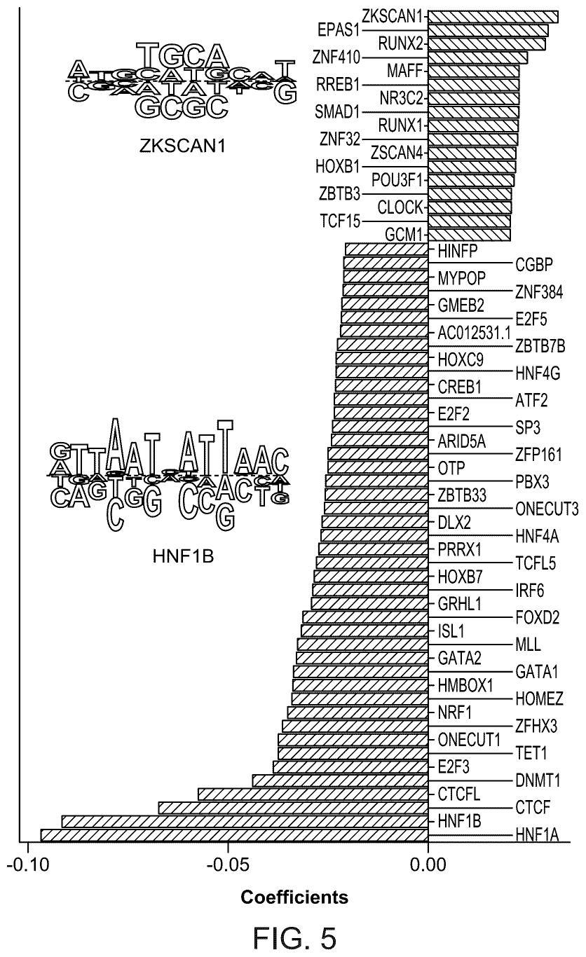

Biological samples (e.g., surgically resected tumors, tumor biopsy, liquid biopsy, circulating shedding tumor cells, circulating tumor DNA) are collected from patients who may be at risk, diagnosed, or suspected of having pancreatic ductal adenocarcinoma (PDAC). The samples are processed for extraction of RNA, optionally and DNA. cDNA samples are generated as described in Example 2 or using conventional methods. The cDNA samples are used as template for amplification of accessible chromatic regions (ACRs) identified in Examples 1 and 2, and quantified by quantitative PCR (qPCR). A set of oligonucleotide probes for targeting differentiated phenotypes (e.g., risk of early recurrence of PDAC) are designed. Each set of oligonucleotide probes include a subset of primers targeting a first phenotype, and a subset of primers targeting a second phenotype. At least one set (e.g., 2, 3, 4, 5, or more) of qPCR primers for each phenotype are designed using publicly available Primer 3 software. The set of primers are designed for targeting 1-100 ACRs associated with recurrent of PDAC as described in Table 1 and/or Table 2, or in FIG. 5. Typically, the set of primers are designed to amplify DNA fragments of no more than 100 bp (e.g., 40-100 bp). Each pair of qPCR primers are placed onto a PCR amplification plate in an array format as shown in FIGS. 4A-4B. The qPCR amplification products are quantified and normalized. Statistical analyses are as described in Example 2. The results show the patient's risk of early recurrence.

### 8.4 Example 4—ATAC-qPCRarray for Predicting Response to Immunotherapy

Biological samples (e.g., surgically resected tumors, tumor biopsy, liquid biopsy, circulating shedding tumor cells, circulating tumor DNA) are collected from patients who may be at risk, diagnosed, or suspected of having pancreatic ductal adenocarcinoma (PDAC). The samples are processed for extraction of RNA, optionally and DNA. cDNA samples are generated as described in Example 2 or using conventional methods. The cDNA samples are used as template for amplification of accessible chromatic regions (ACRs) identified in Examples 1 and 2, and quantified by quantitative PCR (qPCR). At least one set (e.g., 2, 3, 4, 5, or more) of qPCR primers for each phenotype are designed using publicly available Primer 3 software. A set of oligonucleotide probes for targeting differentiated phenotypes (e.g., sensitivity to immunotherapy) are designed. Each set of oligonucleotide probes include a subset of primers targeting a first phenotype, and a subset of primers targeting a second phenotype. The set of primers are designed for targeting 1-100 ACRs associated with sensitivity to immune checkpoint inhibitor therapy (e.g., anti-PD1, anti-PD-L1, nivolumab, pembrolizumab) of PDAC as described in Table 1 and/or Table 2, or in FIG. 5. Typically, the set of primers are designed to amplify DNA fragments of no more than 100 bp (e.g., 40-100 bp). Each pair of qPCR primers are placed onto a PCR amplification plate in an array format as shown in FIGS. 4A-4B. The qPCR amplification products are quantified and normalized. Statistical analyses are as described in Example 2. The results show the patient's sensitivity to immune checkpoint inhibitor therapy and provides assessment for efficacy of personalized immune checkpoint inhibitor therapy.

### 8.5 Example 5—Identification of ATAC-qPCR Array Targets Using Least Absolute Shrinkage and Selection Operator (LASSO)

Ten iterations of 5-fold cross validation were performed within glmnet and the average of the lambda values was chosen as the optimal lambda to control for randomization. Then, for every model, the features with non-zero coefficients were saved and finally compared with the ones resulting from the final model. As a sanity check, the frequency of the features in every iteration's glmnet model was checked and only the features (from the final model) repeating in every single model were chosen as the final results. We used the ATAC-array input data of 49 pancreatic cancer patients with known outcomes (disease-free survival (DFS) following surgery and adjuvant chemotherapy with Gemcitabine/Nab-Paclitaxel) as the training set analyses. Since microarray data are prone to missing values, ATAC-array input data of all 49 patients was filtered for missing values for certain regions before feeding it into the model. Survival data including disease free survival (DFS) and recurrence status of all the 49 patients were used in the model. The following arguments were used as input for the model: family of “Cox” was used, type.measure was set as “C” or Harrel's concordance measure was used for loss of cross-validation, alpha value was set to 1, standardize argument which controls the variable standardization prior to fitting the model sequence was set to TRUE.

The LASSO model resulted in 12 ACRs. Four constitutive ACRs, as described below, were used as controls for normalization of the differential regions. The ACRs were classified as either “blue” regions, or “red” regions, or “green” regions. The “blue” regions indicate the ACRs which are open in patients with good prognosis but silenced (closed) in patient with poor prognosis with Gemcitabine/Nab-Paclitaxel chemotherapy.

Table 3 provides 12 differential and 4 control ACRs identified by analysis in silico.

TABLE 3
Differential and control ACRs identified by analysis in silico

| | | | HUGO | | Length |
|---|---|---|---|---|---|
| Chr | Start | End | symbol | Classification | (bp) |
| chr19 | 4084177 | 4084702 | MAP2K2 | red | 525 |
| chr1 | 56933904 | 56934476 | PPAP2B | red | 572 |
| chr8 | 1878704 | 1879351 | ARHGEF10 | blue | 647 |
| chr3 | 128914473 | 128915151 | CNBP | red | 678 |
| chr6 | 110064994 | 110065287 | FIG4 | red | 293 |
| chr17 | 15917197 | 15917706 | TTC19 | red | 509 |
| chr8 | 8547367 | 8547711 | CLDN23 | blue | 344 |
| chr19 | 39569172 | 39569875 | PAPL | red | 703 |
| chr3 | 152974102 | 152975125 | RN7SL300P | red | 1023 |
| chr2 | 187426114 | 187426881 | ITGAV | red | 767 |
| chr12 | 13539993 | 13540519 | C12orf36 | blue | 526 |
| chr4 | 177114274 | 177114599 | SPATA4 | blue | 325 |
| chr6 | 111279570 | 111280284 | GTF3C6 | ctrl | 714 |
| chr1 | 226270679 | 226271550 | LINC01703 | ctrl | 871 |
| chr1 | 231376653 | 231377603 | C1orf131 | ctrl | 950 |
| chr7 | 152372887 | 152373496 | XRCC2 | ctrl | 609 |

A prognostic score (PS) was assigned to each patient based on these 18 differentially accessible chromatin regions following the formula:

$$Prognostic score ( PS ) by ATAC - array = ( median of blue - median of red ) / median of control green$$

Based on the PS calculated in each patient by this 12-chromatin accessibility signature, patients were segregated by PS values more than and less than the median value respectively, and then analyzed by Cox regression of proportional hazards. FIG. 6 is a K-M graph showing a significant segregation with Log Rank p value=0.0019.

### 8.5.5. Identification of the Internal Control Regions

Intersection analysis of constantly accessible genomic regions from ATAC-Seq data was performed by BEDOPS tool-element-of command (Neph et al., BEDOPS: high-performance genomic feature operations. Bioinformatics. 2012 Jul. 15; 28 (14): 1919-20.) to identify internal control regions. Analysis of data from 336 control peaks from pancreatic tumor cells (Dhara et al., Pancreatic cancer prognosis is predicted by an ATAC-array technology for assessing chromatin accessibility. Nat Commun 12, 3044 (2021).) and 232 control peaks from and CD8+ T cells (Shin, H. M., Kim, G., Kim, S. et al. Chromatin accessibility of circulating CD8+ T cells predicts treatment response to PD-1 blockade in patients with gastric cancer. Nat Commun 12, 975 (2021)) identified 4 peaks that were present in both datasets. While designing qPCR array, these 4 constantly accessible genomic locations were taken as internal control targets to identify differentially accessible regions between pancreatic cancer patient samples of different prognostic outcomes.

### 8.6 Example 6—Designing ATAC-qPCR Array Primers

This example illustrates methods for designing ATAC-qPCR array primers for testing the ACRs identified in Example 5.

The median length of the accessible chromatin regions (ACRs) is 628 bp as shown in Table 3, ranging from minimum of 293, and maximum of 1023. We selected the middle regions (closest to the summit) of all these ACRs, where the odds of representation of these ACRs by the primer oligos are the maximum. The primers were designed by Primer Express™ Software v3.0.1 (cat #4363991). Table 4 shows exemplary primer sequences.

TABLE 4
ATAC-qPCR array primer sequences

| Target | | | | | | | |
|---|---|---|---|---|---|---|---|
| Gene | | Forward | | | | Melting | |
| Regulatory | Target | Primer | SEQ ID | Reverse Primer | SEQ ID | Temperature | Amplicon |
| Region | Coordinates | (5′-3′) | NO. | (5′-3′) | NO. | ° C. | length |
| ARHGEF10 | chr8: | TGGAGTAAG | SEQ ID | TGAGAGGGA | SEQ ID | 87.78 | 114 |
| | 1878704- | CACAGGAGC | NO.: 1 | GGCAACCAG | NO.: 16 | | |
| | 1879351 | CGCT | | AGGC | | | |
| CLDN23 | chr8: | TCCAGCCCA | SEQ ID | TGCTTGGTTG | SEQ ID | 81.13 | 112 |
| | 8547367- | CCTCCTCCC | NO.: 2 | CCAACTCTGC | NO.: 17 | | |
| | 8547711 | AATG | | TC | | | |
| SPATA4 | chr4: | TGCTCAGTTT | SEQ ID | AGTGTGCCAT | SEQ ID | 78.93 | 139 |
| | 177114274- | CATAGGTGA | NO.: 3 | GCTAATTTCC | NO.: 18 | | |
| | 177114599 | CTCA | | TGGA | | | |
| ITGAV | chr2: | TTACCACAG | SEQ ID | TGGAGTCGGG | SEQ ID | 84.6 | 112 |
| | 187426114- | CCCTTGGGC | NO.: 4 | GGAGGGGTA | NO.: 19 | | |
| | 187426881 | GAGA | | ACA | | | |
| FIG4 | chr6: | ACGCTCTGG | SEQ ID | AGACACAGTG | SEQ ID | 81.64 | 110 |
| | 110064994- | GAAGCCTCT | NO.: 5 | AGCTGTCCCA | NO.: 20 | | |
| | 110065287 | TCCT | | GC | | | |
| MAP2K2 | chr19: | GGCCGCTGC | SEQ ID | GGGCTCCAGG | SEQ ID | 85.83 | 130 |
| | 4084177- | TGGTTTCGTT | NO.: 6 | GCAGGTTAAG | NO.: 21 | | |
| | 4084702 | TTG | | GA | | | |
| PPAP2B | chr1: | GGCTTCACT | SEQ ID | AGCTGACGGG | SEQ ID | 80.87 | 114 |
| | 35693904- | GGTGACCAA | NO.: 7 | AGAGGCTTTC | NO.: 22 | | |
| | 56934476 | GACAGC | | GT | | | |
| TTC19 | chr17: | GCAGTTCAG | SEQ ID | TCAACCACCA | SEQ ID | 80.31 | 132 |
| | 15917197- | CAATAGGGA | NO.: 8 | GGGGAGCAT | NO.: 23 | | |
| | 15917706 | GCAGGC | | GGG | | | |
| RN7SL300P | chr3: | CTCTGTTGCC | SEQ ID | GGCCCTTTGT | SEQ ID | 80.72 | 102 |
| | 152974102- | CAGGCTGGA | NO.: 9 | CGAAACGTTT | NO.: 24 | | |
| | 152975125 | GTG | | GCA | | | |
| PAPL | chr19: | TTTGGGAGG | SEQ ID | TGTCACCCAG | SEQ ID | 74.43 | 101 |
| | 39569172- | CTGAGGTGG | NO.: 10 | GCTGGAGTGC | NO.: 25 | | |
| | 39569875 | GAGG | | AT | | | |
| CNBP | chr3: | ACCAGCCCT | SEQ ID | GCATGCTCTG | SEQ ID | 81.33 | 120 |
| | 128914473- | CGGAAACTG | NO.: 11 | TGGCTGGCTG | NO.: 26 | | |
| | 128915151 | CTGA | | AA | | | |
| RP11 | chr1: | TCCCCAGCA | SEQ ID | TTCTCTCTGG | SEQ ID | 88.4 | 138 |
| | 226270679- | TCTCTCCAG | NO.: 12 | CACCTGGGGC | NO.: 27 | | |
| | 226271550 | CGTC | | TG | | | |
| XRCC2 | chr7: | TCACCTCGG | SEQ ID | GCGCAGTTGG | SEQ ID | 86.27 | 109 |
| | 152372887- | TCCCAGACT | NO.: 13 | TGAATGGCGT | NO.: 28 | | |
| | 152373496 | CAGC | | TG | | | |
| C1orf131 | chr1: | CAGCCGCAC | SEQ ID | GGGTACTATT | SEQ ID | 87.54 | 140 |
| | 231376653- | CATGGAGTC | NO.: 14 | TCGCGCGGCT | NO.: 29 | | |
| | 231377603 | TTCC | | CC | | | |
| GTF3C6 | chr6: | CTGGCAGAA | SEQ ID | GCCTTCTAGT | SEQ ID | 89.01 | 125 |
| | 111279570- | GCAATTTGC | NO.: 15 | CACTGGCCAC | NO.: 30 | | |
| | 111280284 | GCGG | | GC | | | |

The first 3 primers (ARGFEF10, CLDN23, and SPATA4) were specific to three chromatin cis-regulatory regions that are differentially accessible to good prognosis patients (“blue” regions), the next set of 8 primers (MAPK2, PPAP2B, CNBP, FIG. 4, TTC19, PAPL, RN7SL, and ITGAV) were specific to 8 chromatin cis-regulatory regions that are differentially accessible to poor prognosis patients (“red” regions), and the remaining 4 primers (GTF3C6, C1orf131, XRCC2, and RP11) were specific to 4 chromatin cis-regulatory regions that are constitutively accessible to all patients (“green” regions).

All these custom-designed oligonucleotides sequences (primer pairs as displayed in Table 2) were first tested for specificity in silico by NCBI Basic Local Alignment Tool (BLAST), and then the oligonucleotides were purchased from IDT (Integrated DNA Technogies, Coralville, IA).

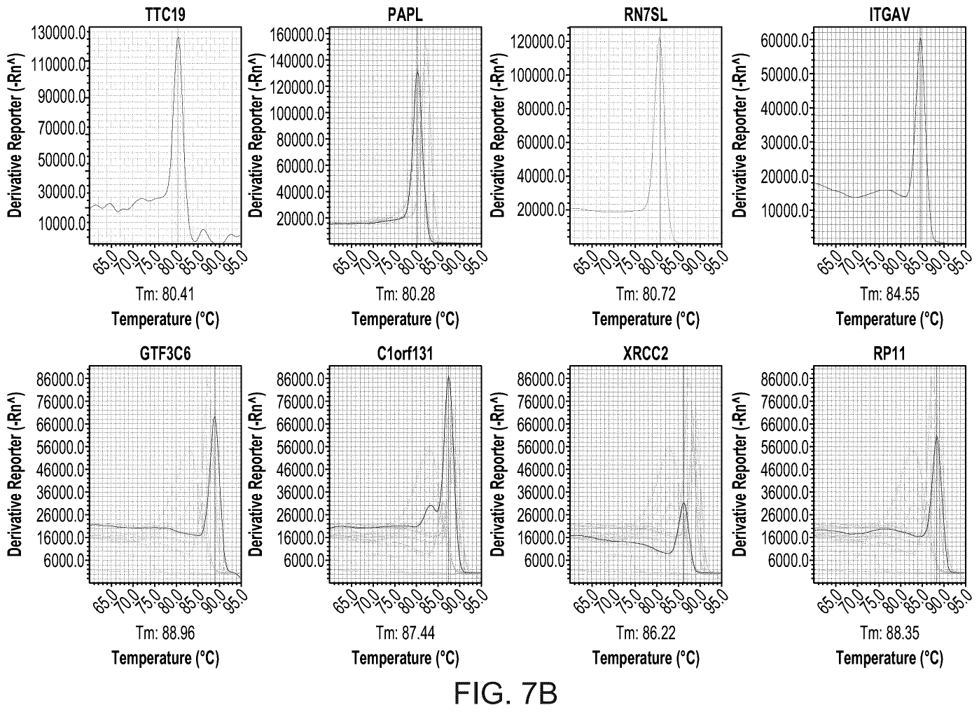

FIGS. 7A and 7B provide results from melting curve analysis showing a single peak for each primer set. Quantitative measurement of target accessible region relative to control regions were performed in triplicates using Power SYBR Green PCR Master Mix (Applied Biosystems, Warrington, UK cat #4368706) following the manufacturer's recommendations. All the qPCR experiments were run on QuantStudio™ 3 Real-Time PCR System, 96-well, 0.2 mL (Applied Biosystem cat #A28567). Commercially available male genomic DNA (Agilent Technologies cat #5190-4370) were used as the template to test the performances of all the primer sets. The single products were tested by Melting curve analysis as shown in FIGS. 7A and 7B, each amplicon showed only one peak at specific temperature (as listed in Table 4) confirming single product per primer set. The specificity of the primer sets was confirmed by Sanger sequencing analysis of the amplicons.

### 8.7 Example 7—Determining a Prognostic Score of a Patient Using ATAC-qPCR Array Primers

This example provides a method for determining prognostic score of a patient using ATAC-qPCR array primers described in Example 6.

We selected 14 patients with pancreatic cancer who had undergone upfront surgery followed by Gemcitabine/Nab-Paclitaxel adjuvant chemotherapy. These patients were monitored for a median follow up time of 4.15 years post-surgery. Seven (7) patients showed good prognosis with a median disease-free survival (DFS) of 1478 days (ranging from 1234 to 1645), and the other 7 patients showed poor prognosis with a median DFS of 47 days (ranging from 36 to 207) with the similar treatment regimen. FIG. 8 shows the Kaplan-Meier graph showing patient with good prognosis and poor prognosis. We first estimated the relative accessibility of the differentially accessible chromatin regions (the blue, and red regions, normalized with the 4 green regions) by 2\hat{ }⁻dCt method in each patient by qPCR analyses of the individual ATAC-libraries prepared from them. The dCt values for each target region was calculated as below:

dCt=Ct values of the differential (blue or red) regions−Geometric mean Ct values of the control (green) regions

FIGS. 9A and 9B show the results of ATAC-qPCR analysis of the 11 target regions using the 4 control regions for normalization. The relative quantitation of blue and red regions in each individual patient were shown by bar plot, where the blue bars represented the blue target regions and the red bars represented the red target regions.

Next, we calculated the Prognostic scores using the ATAC-qPCR analytical methods as displayed below:

Prognostic score (PS) by ATAC-qPCR array=2\hat{ }⁻dCt of blue regions−2\hat{ }⁻dCt of red regions

Using the formula described above we calculated the individualized PS for each patient and analyzed the PS of good and poor prognosis groups. The box plot as displayed in FIG. 10 demonstrated a statistically significant difference (t test p=0.034) of PS between the 7 good and 7 poor prognosis patients.

The data demonstrate that a set of targeting oligonucleotide probes required for effectively determining a reliable prognostic score can be less than 100, or as few as 12 probes. The set of targeting oligonucleotide probes required for effectively determining good prognosis can be as few as 3 probes, and the set of targeting oligonucleotide probes required for effectively determining poor prognosis can be as few as 8 probes. The result demonstrate that ATAC-qPCR array is a robust, reliable and cost-effective method for determining prognostic score of a patient having, or at risk of developing pancreatic cancer. Such information is critical for the selection of treatment modalities, which can enhance the subject's likelihood of disease-free survival.

### 9. EMBODIMENTS

Embodiment 1. A method for determining an epigenetic landscape associated with a specific phenotypic trait of a biological sample, the method comprising:

providing a biological sample comprising morphologically intact nuclei or intact nucleosome (histone-DNA) structure;

contacting the intact nuclei or intact nucleosome to a transposase complex to produce a population of tagged DNA fragments representing accessible chromatin regions (ACRs) of the intact nuclei or intact nucleosome (histone-DNA) structure;

hybridizing a set of targeting oligonucleotide probes to a specific region on the ACRs to generate targeted ACR fragments, wherein the targeting oligonucleotide probes specifically targets no more than 100 differentially accessible chromatin regions selected from Table 1 or Table 2, wherein the targeting oligonucleotide probes comprise:
a first subset of oligonucleotide probes targeting ACRs associated with a first phenotype, and
a second subset of oligonucleotide probes targeting ACRs associated with a second phenotype;

amplifying the targeted ACR fragments associated with the first phenotype and the second phenotype obtained in (c); and

quantifying the amount of amplified targeted ACR fragments, thereby determining the epigenetic landscape associated with the first and the second phenotypic traits of the biological sample.

Embodiment 2. The method of embodiment 1, wherein the amplification fragment comprises a mean size of less than 100 bp.

Embodiment 3. The method of embodiment 1 or embodiment 2, wherein the first phenotype is recurrence of a cancer within one year of surgical resection and the second phenotype is non-recurrence of a cancer within one year of surgical resection.

Embodiment 4. The method of embodiment 1 or embodiment 2, wherein the first phenotype is non-responder and the second phenotype is responder to a cancer therapy.

Embodiment 5. The method of embodiment 4, wherein the cancer therapy is selected from chemotherapy, immunotherapy, and radiation.

Embodiment 6. The method of any one of embodiments 1-5, further comprising assessing nuclear localization of one or more biomarkers capable of modulating gene expression through complementary binding to one or more specific regions on the amplified targeted ACR fragments.

Embodiment 7. The method of embodiment 6, wherein the biomarker is a transcription factor.

Embodiment 8. The method of embodiment 7, wherein the transcription factor is selected from ZKSCAN1, EPAS1, RUNX2, ZNF410, MAFF, RREB1, NR3C2, SMAD1, RUNX1, ZNF32, ZSCAN4, HOXB1, POU3F1, ZBTB3, CLOCK, TCF15, GCM1, HINFP, CGBP, MYPOP, ZNF384, GMEB2, E2F5, AC012531.1, ZBTB7B, HOXC9, HNF4G, CREB1, ATF2, E2F2, SP3, ARID5A, ZFP161, OTP, PBX3, ZBTB33, ONECUT3, ONECUT3, DLX2, HNF4A, PRRX1, TCFL5, HOXB7, IRF6, GRHL1, FOXD2, ISL1, MLL, GATA2, GATA1, HMBOX1, NRF1, ZFHX3, ONECUT1, TET1, E2F3, DNMT1, CTCFL, CTCF, HNF1B, and HNF1A.

Embodiment 9. The method of any one of embodiments 1-8, wherein the biological sample is selected from a tumor biopsy, surgically resected tumor specimen, or liquid biopsy.

Embodiment 10. The method of any one of embodiments 1-8, wherein the biological sample is pancreatic ductal adenocarcinoma tissue.

Embodiment 11. The method of embodiment 9 or embodiment 10, wherein the biological sample comprises circulating shedding tumor cells or circulating tumor DNA (ctDNA).

Embodiment 12. The method of embodiment 9 or embodiment 10, wherein the biological sample comprises treatment-naïve malignant cells.

Embodiment 13. The method of embodiment 9 or embodiment 10, wherein the biological sample is collected from a patient who had been treated with one or more treatment modalities.

Embodiment 14. The method of any one of embodiments 1-13, wherein the phenotypic trait is responsiveness to a treatment modality.

Embodiment 15. The method of embodiment 13 or embodiment 14, wherein the treatment modality is selected from the group consisting of surgical resection, chemotherapy, radiation, immunotherapy, and a combination thereof.

Embodiment 16. The method of any one of embodiments 1-15, further comprising isolating nucleosomal DNA from the cell nuclei of the biological sample.

Embodiment 17. The method of embodiment 16, wherein the nuclei are isolated and in a manner that maintains nucleosome structure.

Embodiment 18. The method of any one of embodiments 1-17, wherein the method does not include sequencing the tagged fragments or amplicons thereof.

### 10. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes. In particular, PCT International Patent Application No: PCT/US2019/046301, U.S. patent application Ser. No. 17/268,195, U.S. patent application Ser. No. 17/324,093, and Dhara et al., Pancreatic cancer prognosis is predicted by an ATAC-array technology for assessing chromatin accessibility. Nat Commun 12, 3044 (2021), are each hereby incorporated by reference in their entireties.

1. A method for determining a prognostic score indicative of a subject's responsiveness to one or more treatment modalities or indicative of a duration of disease-free survival, the method comprising:
(a) contacting a biological sample obtained from the subject with a transposase complex to produce a population of tagged DNA fragments representing accessible chromatin regions of intact nuclei or intact nucleosome structure, wherein the biological sample comprises pancreatic ductal adenocarcinoma cells having morphologically intact nuclei or intact nucleosomes;
(b) hybridizing a set of targeting oligonucleotide probes to a specific region on the accessible chromatin regions to generate targeted accessible chromatin region fragments, wherein the targeting oligonucleotide probes specifically target differentially accessible chromatin regions, and wherein the targeting oligonucleotide probes comprise:
(i) a first subset of oligonucleotide probes targeting accessible chromatin regions associated with a first phenotype, and
(ii) a second subset of oligonucleotide probes targeting accessible chromatin regions associated with a second phenotype;
(c) amplifying the targeted accessible chromatin region fragments obtained in (b) that are associated with the first phenotype and the second phenotype; and
(d) determining a prognostic score based on epigenetic signature values of the biological sample,
wherein the prognostic score is determined based on a differential of a first epigenetic signature value and a second epigenetic signature value, and
wherein the prognostic score is indicative of the subject's responsiveness to the one or more treatment modalities or indicative of a duration of disease-free survival.

2. The method of claim 1, further comprising, prior to step (a), enriching the biological sample for tumor cells.

3. The method of claim 1, further comprising contacting the biological sample with an agent to isolate tumor cells from non-tumor cells in the biological sample to enrich the sample for tumor cells.

4. The method of claim 3, wherein the agent comprises antibody-conjugated magnetic beads, EpCAM-conjugated magnetic beads, or a combination thereof.

5. The method of claim 1, further comprising:
(i) attaching a detectable label to the tagged DNA fragments to produce labeled fragments; and
(ii) contacting the detectable labeled fragments to the set of targeting oligonucleotide probes.

6. The method of claim 1, further comprising quantifying the amount of amplified targeted accessible chromatin region fragments to obtain the first epigenetic signature value and the second epigenetic value.

7. The method of claim 1, wherein the set of targeting oligonucleotide probes are selected from any pair of oligonucleotide probes in Table 4.

8. The method of claim 1, wherein the targeting oligonucleotide probes comprises a nucleic acid sequence having a sequence of any one of SEQ ID NOs.: 1-30.

9. The method of claim 1, wherein the set of targeting oligonucleotide probes comprises at least one set of probes targeting ARHGEF10, CLDN23, C12orf36, and SPATA4.

10. The method of claim 1, wherein the set of targeting oligonucleotide probes comprises at least one set of probes targeting MAP2K2, PPAP2B, CNBP, FIG. 4, TTC19, PAPL, RN7SL300P, and ITGAV.

11. The method of claim 1, wherein the set of targeting oligonucleotide probes comprises at least one set of probes targeting GTF3C6, LINC01703, C1orf131, and XRCC2.

12. The method of claim 1, wherein the accessible chromatin regions are selected from any one of the accessible chromatin regions in FIG. 9A, FIG. 9B, and/or Table 3.

13. The method of claim 12, wherein the accessible chromatin regions are selected from any one of the accessible chromatin regions in Table 3.

14. The method of claim 1, further comprising the step of comparing the first epigenetic signature value to the second epigenetic signature value to obtain a differential value.

15. The method of claim 1, further comprising normalizing the differential value with at least one of a positive control value and one of a negative control value to obtain the prognostic score.

16. The method of claim 15, wherein the prognostic score is at least 0.6.

17. The method of claim 16, further comprising detecting nuclear localization of at least one of a transcription factor selected from any one of the transcription factors in Tables 2A and 2B.

18. The method of claim 17, wherein the transcription factors are selected from ZKSCAN1, EPAS1, RUNX2, ZNF410, MAFF, RREB1, NR3C2, SMAD1, RUNX1, ZNF32, ZSCAN4, HOXB1, POU3F1, ZBTB3, CLOCK, TCF15, GCM1, HINFP, CGBP, MYPOP, ZNF384, GMEB2, E2F5, AC012531.1, ZBTB7B, HOXC9, HNF4G, CREB1, ATF2, E2F2, SP3, ARID5A, ZFP161, OTP, PBX3, ZBTB33, ONECUT3, ONECUT3, DLX2, HNF4A, PRRX1, TCFL5, HOXB7, IRF6, GRHL1, FOXD2, ISL1, MLL, GATA2, GATA1, HMBOX1, NRF1, ZFHX3, ONECUT1, TET1, E2F3, DNMT1, CTCFL, CTCF, HNF1B, and HNF1A.

19. The method of claim 18, wherein the transcription factors are selected from ZKSCAN1A, HNF1B, or a combination thereof.

20. The method of claim 1, further comprising predicting a long duration of disease-free survival when the first second epigenetic signature value is significantly higher than the second first epigenetic signature value and/or predicting a short duration of disease-free survival when the second first epigenetic signature value is significantly higher than the first second epigenetic value.

21. The method of claim 1, wherein the first phenotype is recurrence of a cancer within one year of surgical resection and the second phenotype is non-recurrence of a cancer within one year of surgical resection.

22. The method of claim 1, wherein the first phenotype is non-responder and the second phenotype is responder to a cancer therapy.

23. The method of claim 22, wherein the cancer therapy is selected from chemotherapy, immunotherapy, radiation, or combinations thereof.

24. The method of claim 1, wherein the second phenotype is having a median disease-free survival of between 50 to 1500 days.

25.-27. (canceled)

28. The method of claim 1, wherein the biological sample comprises treatment-naïve malignant cells.

29. The method of claim 28, wherein the subject is a treatment naïve patient who has not received the one or more treatment modalities.

30. The method of claim 1, wherein the subject is under treatment or has been treated with the one or more treatment modalities.

31. The method of claim 1, wherein the one or more treatment modalities are selected from resecting cancerous tissue, neo-adjuvant chemotherapy, adjuvant chemotherapy, immunotherapy, an epigenetic drug, or combinations thereof.

32. The method of claim 31, wherein the epigenetic drug is selected from DNMT inhibitor, an HDAC inhibitor, an EZH2 inhibitor, or combinations thereof.

33. The method of claim 32, wherein the neo-adjuvant chemotherapy and/or adjuvant chemotherapy is selected from gemcitabine, nab-paclitaxel, fluorouracil (5-FU), irinotecan, oxaliplatin, leucovorin, capecitabine, cisplatin, or combinations thereof.

34. The method of claim 1, wherein the biological sample is selected from a tumor biopsy or surgically resected tumor specimen.

35. The method of claim 1, wherein the method does not include sequencing the tagged fragments or amplicons thereof.

36. The method of claim 1, wherein the amplified targeted accessible chromatin fragments comprise a mean size of about 120 bp.

37. A method of treating a subject having, or suspected of having, pancreatic ductal adenocarcinoma with one or more treatment modalities, the method comprising:
(a) receiving a prognostic score indicative of the subject's responsiveness to one or more treatment modalities or indicative of a duration of disease-free survival,
wherein the prognostic score is determined based on epigenetic signature values of a biological sample from the subject that comprises pancreatic ductal adenocarcinoma cells having morphologically intact nuclei or intact nucleosomes, the biological sample having been contacted with a transposase complex to produce a population of tagged DNA fragments representing accessible chromatin regions of the intact nuclei or intact nucleosome structure,
wherein a set of targeting oligonucleotide probes were hybridized to a specific region on the accessible chromatin regions to generate targeted accessible chromatin region fragments,
wherein the targeting oligonucleotide probes specifically target differentially accessible chromatin regions,
wherein the prognostic score is determined based on a differential value of a first epigenetic signature value and a second epigenetic signature value; and
(b) treating the subject with the one or more treatment modalities based on the prognostic score.

38. The method of claim 37, wherein the biological sample is, prior to step (a), enriched for tumor cells.

39. The method of claim 38, wherein the enriched tumor cells are obtained by contacting the biological sample with an agent to isolate tumor cells from non-tumor cells in the biological sample to enrich the sample for tumor cells.

40. The method of claim 39, wherein the agent comprises antibody-conjugated magnetic beads, EpCAM-conjugated magnetic beads, or a combination thereof.

41. The method of claim 37, wherein the biological sample is obtained by:
(i) attaching a detectable label to the tagged DNA fragments to produce labeled fragments; and
(ii) contacting the detectable labeled fragments to the set of targeting oligonucleotide probes.

42. The method of claim 37, wherein the first epigenetic signature value and the second epigenetic signature value are obtained by quantifying the amount of amplified targeted accessible chromatin region fragments.

43. The method of claim 37, wherein the set of targeting oligonucleotide probes are selected from any pair of oligonucleotide probes in Table 4.

44. The method of claim 37, wherein the targeting oligonucleotide probes comprises a nucleic acid sequence having a sequence of any one of SEQ ID NOs.: 1-30.

45. The method of claim 37, wherein the set of targeting oligonucleotide probes comprises at least one set of probes targeting ARHGEF10, CLDN23, C12orf36, and SPATA4.

46. The method of claim 37, wherein the set of targeting oligonucleotide probes comprises probes targeting at least one of MAP2K2, PPAP2B, CNBP, FIG. 4, TTC19, PAPL, RN7SL300P, and ITGAV.

47. The method of claim 37, wherein the set of targeting oligonucleotide probes comprises at least one set of probes targeting GTF3C6, LINC01703, C1orf131, and XRCC2.

48. The method of claim 37, wherein the accessible chromatin regions are selected from any one of the accessible chromatin regions in FIG. 9A, FIG. 9B, and/or Table 3.

49. The method of claim 48, wherein the accessible chromatin regions are selected from any one of the accessible chromatin regions in Table 3.

50. The method of claim 37, wherein determining the prognostic score comprises the step of comparing the first epigenetic signature value to the second epigenetic signature value to obtain the differential value.

51. The method of claim 37, wherein determining the prognostic score comprises the step of normalizing the differential value with at least one of a positive control value and a negative control value to obtain the prognostic score.

52. The method of claim 51, wherein the prognostic score is at least 0.6.

53. The method of claim 52, wherein determining the prognostic score comprises the step of comprises detecting nuclear localization of a transcription factor selected from any one of transcription factors in Tables 2A and 2B.

54. The method of claim 53, wherein the transcription factors are selected from ZKSCAN1, EPAS1, RUNX2, ZNF410, MAFF, RREB1, NR3C2, SMAD1, RUNX1, ZNF32, ZSCAN4, HOXB1, POU3F1, ZBTB3, CLOCK, TCF15, GCM1, HINFP, CGBP, MYPOP, ZNF384, GMEB2, E2F5, AC012531.1, ZBTB7B, HOXC9, HNF4G, CREB1, ATF2, E2F2, SP3, ARID5A, ZFP161, OTP, PBX3, ZBTB33, ONECUT3, ONECUT3, DLX2, HNF4A, PRRX1, TCFL5, HOXB7, IRF6, GRHL1, FOXD2, ISL1, MLL, GATA2, GATA1, HMBOX1, NRF1, ZFHX3, ONECUT1, TET1, E2F3, DNMT1, CTCFL, CTCF, HNF1B, and HNF1A.

55. (canceled)

56. The method of claim 37, wherein determining the prognostic score comprises the step of predicting a long duration of disease-free survival when the second epigenetic signature value is significantly higher than the first epigenetic signature value and/or predicting a short duration of disease-free survival when the first epigenetic signature value is significantly higher than the first epigenetic value.

57. The method of claim 37, wherein the first phenotype is recurrence of a cancer within one year of surgical resection and the second phenotype is non-recurrence of a cancer within one year of surgical resection.

58. The method of claim 37, wherein the second phenotype is responder and the first phenotype is non-responder to a cancer therapy.

59. The method of claim 58, wherein the cancer therapy is selected from chemotherapy, immunotherapy, radiation, or combinations thereof.

60. The method of claim 37, wherein the second phenotype is having a median disease-free survival of between 50 to 1500 days.

61.-63. (canceled)

64. The method of claim 37, wherein the biological sample comprises treatment-naïve malignant cells.

65.-66. (canceled)

67. The method of claim 37, wherein the one or more treatment modalities are selected from resecting cancerous tissue, neo-adjuvant chemotherapy, adjuvant chemotherapy, immunotherapy, an epigenetic drug, or combinations thereof.

68. The method of claim 67, wherein the epigenetic drug is selected from DNMT inhibitor, an HDAC inhibitor, an EZH2 inhibitor, or combinations thereof.

69. The method of claim 68, wherein the neo-adjuvant chemotherapy and/or adjuvant chemotherapy is selected from gemcitabine, nab-paclitaxel, fluorouracil (5-FU), irinotecan, oxaliplatin, leucovorin, capecitabine, cisplatin, or combinations thereof.

70. The method of claim 37, wherein the biological sample is selected from a tumor biopsy or surgically resected tumor specimen.

71. The method of claim 37, wherein obtaining the targeted accessible chromatin region fragments does not include the step of sequencing the tagged fragments or amplicons thereof.

72. (canceled)

73. The method of claim 1, wherein the set of targeting oligonucleotide probes required for effectively determining the prognostic score is between 3-100.

74. The method of claim 73, wherein the set of targeting oligonucleotide probes required for effectively determining the prognostic score is 12.

75. (canceled)

76. The method of claim 75, wherein the set of targeting oligonucleotide probes required for effectively determining good prognosis is 3.

77. The method of claim 76, wherein the set of targeting oligonucleotide probes required for effectively determining poor prognosis is 8.

78. A kit for determining an epigenetic landscape associated with a specific phenotypic trait of a biological sample in accordance with the method in claim 1, the kit comprising:
(a) an array configured to detect targeted accessible chromatin region fragments obtained from the biological sample, wherein the array comprises a panel of sets of targeting oligonucleotide probes specifically targeting differentially accessible chromatin regions;
(b) reagents; and
(c) instructions for amplifying and quantifying the targeted accessible chromatin region fragments to obtain a first epigenetic signature value and a second epigenetic value.

79. (canceled)

80. A kit for determining an epigenetic landscape associated with a specific phenotypic trait of a biological sample in accordance with the method in claim 1, the kit comprising:
(a) one or more sets of targeting oligonucleotide probes for detect targeted accessible chromatin region fragments obtained from the biological sample, wherein the targeting oligonucleotide probes specifically target and amplify differentially accessible chromatin regions to generate one or more targeted accessible chromatin region fragments;
(b) reagents; and
(c) instructions for amplifying and quantifying the targeted accessible chromatin region fragments to obtain a first epigenetic signature value and a second epigenetic value.

81.-118. (canceled)


SEQUENCE LISTING (30 sequences)

SEQ ID NO: 1 - DNA, 22 bp, synthetic construct
  source 1..22: mol_type = other DNA
tggagtaagcacaggagccgct

SEQ ID NO: 2 - DNA, 22 bp, synthetic construct
  source 1..22: mol_type = other DNA
tccagcccacctcctcccaatg

SEQ ID NO: 3 - DNA, 23 bp, synthetic construct
  source 1..23: mol_type = other DNA
tgctcagtttcataggtgactca

SEQ ID NO: 4 - DNA, 22 bp, synthetic construct
  source 1..22: mol_type = other DNA
ttaccacagcccttgggcgaga

SEQ ID NO: 5 - DNA, 22 bp, synthetic construct
  source 1..22: mol_type = other DNA
acgctctgggaagcctcttcct

SEQ ID NO: 6 - DNA, 22 bp, synthetic construct
  source 1..22: mol_type = other DNA
ggccgctgctggtttcgttttg

SEQ ID NO: 7 - DNA, 24 bp, synthetic construct
  source 1..24: mol_type = other DNA
ggcttcactggtgaccaagacagc

SEQ ID NO: 8 - DNA, 24 bp, synthetic construct
  source 1..24: mol_type = other DNA
gcagttcagcaatagggagcaggc

SEQ ID NO: 9 - DNA, 22 bp, synthetic construct
  source 1..22: mol_type = other DNA
ctctgttgcccaggctggagtg

SEQ ID NO: 10 - DNA, 22 bp, synthetic construct
  source 1..22: mol_type = other DNA
tttgggaggctgaggtgggagg

SEQ ID NO: 11 - DNA, 22 bp, synthetic construct
  source 1..22: mol_type = other DNA
accagccctcggaaactgctga

SEQ ID NO: 12 - DNA, 22 bp, synthetic construct
  source 1..22: mol_type = other DNA
tccccagcatctctccagcgtc

SEQ ID NO: 13 - DNA, 22 bp, synthetic construct
  source 1..22: mol_type = other DNA
tcacctcggtcccagactcagc

SEQ ID NO: 14 - DNA, 22 bp, synthetic construct
  source 1..22: mol_type = other DNA
cagccgcaccatggagtcttcc

SEQ ID NO: 15 - DNA, 22 bp, synthetic construct
  source 1..22: mol_type = other DNA
ctggcagaagcaatttgcgcgg

SEQ ID NO: 16 - DNA, 22 bp, synthetic construct
  source 1..22: mol_type = other DNA
tgagagggaggcaaccagaggc

SEQ ID NO: 17 - DNA, 22 bp, synthetic construct
  source 1..22: mol_type = other DNA
tgcttggttgccaactctgctc

SEQ ID NO: 18 - DNA, 24 bp, synthetic construct
  source 1..24: mol_type = other DNA
agtgtgccatgctaatttcctgga

SEQ ID NO: 19 - DNA, 22 bp, synthetic construct
  source 1..22: mol_type = other DNA
tggagtcgggggaggggtaaca

SEQ ID NO: 20 - DNA, 22 bp, synthetic construct
  source 1..22: mol_type = other DNA
agacacagtgagctgtcccagc

SEQ ID NO: 21 - DNA, 22 bp, synthetic construct
  source 1..22: mol_type = other DNA
gggctccagggcaggttaagga

SEQ ID NO: 22 - DNA, 22 bp, synthetic construct
  source 1..22: mol_type = other DNA
agctgacgggagaggctttcgt

SEQ ID NO: 23 - DNA, 22 bp, synthetic construct
  source 1..22: mol_type = other DNA
tcaaccaccaggggagcatggg

SEQ ID NO: 24 - DNA, 23 bp, synthetic construct
  source 1..23: mol_type = other DNA
ggccctttgtcgaaacgtttgca

SEQ ID NO: 25 - DNA, 22 bp, synthetic construct
  source 1..22: mol_type = other DNA
tgtcacccaggctggagtgcat

SEQ ID NO: 26 - DNA, 22 bp, synthetic construct
  source 1..22: mol_type = other DNA
gcatgctctgtggctggctgaa

SEQ ID NO: 27 - DNA, 22 bp, synthetic construct
  source 1..22: mol_type = other DNA
ttctctctggcacctggggctg

SEQ ID NO: 28 - DNA, 22 bp, synthetic construct
  source 1..22: mol_type = other DNA
gcgcagttggtgaatggcgttg

SEQ ID NO: 29 - DNA, 22 bp, synthetic construct
  source 1..22: mol_type = other DNA
gggtactatttcgcgcggctcc

SEQ ID NO: 30 - DNA, 22 bp, synthetic construct
  source 1..22: mol_type = other DNA
gccttctagtcactggccacgc